(19) United States
(12) Patent Application Publication
(10) Pub. No.: US 2015/0184159 A1
(43) Pub. Date: Jul. 2, 2015

(54) USE OF COX-3 BINDING MOLECULES FOR MODULATING AUTOPHAGY

(71) Applicant: Brigham Young University, Provo, UT (US)

(72) Inventors: Daniel Lewis Simmons, Provo, UT (US); Jonathan James Lee, Provo, UT (US); John Cameron Hunter, Rowlett, TX (US); Gideon Logon, Draper, UT (US)

(21) Appl. No.: 14/505,338

(22) Filed: Oct. 2, 2014

Related U.S. Application Data
(60) Provisional application No. 61/960,988, filed on Oct. 2, 2013.
(60) Provisional application No. 61/996,944, filed on May 19, 2014.

(51) Int. Cl. C12N 15/113; C07K 16/40; C12Q 1/26

(52) U.S. Cl. CPC C12N 15/1137; C12Q 1/26; C07K 16/40; C12N 2310/14; C07K 2317/76; G01N 2333/90245; G01N 2500/20; G01N 2500/04

(57) ABSTRACT

The invention provides method for modulating autophagy in a cell comprising contacting one or more cells that expresses COX-3 with an effective amount of an agent that modulates the expression level and/or enzymatic activity of COX-3 or a isoform thereof. The invention further provides for inhibiting viral replication (e.g., autophagy-associated viral infection) comprising contacting a cell that expresses COX-3 with an effective amount of an agent that inhibits the expression level and/or enzymatic activity of COX-3 or isoform thereof. Further provided are kits and articles of manufacture comprising inhibitors of the level and/or activity of COX-3 or isoform thereof. Methods for screening for modulators of the level and/or activity of COX-3 or isoform thereof are also provided.

### CROSS-REFERENCE TO RELATED APPLICATIONS

This application claims the benefit of U.S. Provisional Application Ser. No. 61/960,988, filed on Oct. 2, 2013, and U.S. Provisional Application Ser. No. 61/996,944, filed on May 19, 2014, the contents of which are hereby incorporated by reference in their entirety.

### BACKGROUND OF THE INVENTION

In eukaryotic cells, polyunsaturated fatty acids are oxygenated by three general systems: 1) cyclooxygenases (COXs) and related fatty acid oxygenases, including pathogen-inducible oxygenases (PIOXs) identified in plants, animals and bacteria; 2) lipoxygenases; and 3) cytochrome P-450. Presently there are two COX isozymes known, COX-1 and COX-2.

A new variant of the two COX isozymes, termed COX-3 or COX-1b, has also been discovered. COX-3 is an enzymatically active prostaglandin synthase and possesses distinct pharmacological properties relative to COX-1 and COX-2. COX-3 mRNA is expressed at relatively high levels in a tissue- and cell-type dependent manner in all species examined. COX-3 mRNA encodes multiple large molecular weight cyclooxygenase-like proteins from the same reading frame as COX-1. The size and cellular location of these proteins suggests potential roles as cytosolic enzymes and nuclear factors.

The cyclooxygenation of arachidonic acid, catalyzed by two forms of cyclooxygenase, produces prostaglandins which regulate neurotransmission and immune and inflammatory responses. (Goetzl et al., FASEB J., 9:1051, 1995). Inflammation, for instance, is both initiated and maintained, at least in part, by the overproduction of prostaglandins in injured cells. The central role that prostaglandins play in inflammation is underscored by the fact that those aspirin-like non-steroidal anti-inflammatory drugs (NSAIDS) that are most effective in the therapy of many pathological inflammatory states all act by inhibiting prostaglandin synthesis. NSAIDs are analgesic/anti-inflammatory/antipyretic medications that act as inhibitors of the cyclooxygenase active site of COX isozymes. Important mechanistic differences in the actions of individual NSAIDs with the COX active site exist. Of the NSAIDs in medical use, only aspirin is a covalent modifier of COX-1 and COX-2.

There is, therefore, a need to continue developing compounds that modulate cyclooxygenase activity and methods for identifying such compounds.

### SUMMARY OF THE INVENTION

The invention relates at least in part to methods for using modulators of COX-3 for modulating of autophagy.

According to aspects of the invention illustrated herein, there is provided a method for modulating autophagy in a cell including contacting one or more cells that expresses COX-3 with an effective amount of an agent that modulates the expression level and/or enzymatic activity of COX-3 or a component thereof.

According to aspects illustrated herein, there is provided a method for inhibiting encephalomyocarditis viral (EMCV) replication comprising contacting a cell that expresses COX-3 with an effective amount of an agent that inhibits the expression level and/or enzymatic activity of COX-3 or a component thereof.

According to aspects illustrated herein, there is provided a method for inhibiting viral infection in a subject comprising contacting a cell that expresses COX-3 with an effective amount of an agent that increases the expression level and/or enzymatic activity of COX-3 or a component thereof.

According to aspects illustrated herein, there is provided a method of identifying a candidate agent that modulates autophagy in a cell comprising: a) contacting a cell or population of cells that expresses COX-3 protein with a candidate autophagy modulating agent; and b) measuring the level of expression and/or enzymatic activity of COX-3, wherein: i) a decrease in expression and/or enzymatic activity of COX-3 protein relative to a control cell or population of cells not exposed to said candidate autophagy modulating agent is indicative that said candidate autophagy modulating agent inhibits autophagy; or ii) an increase in expression and/or enzymatic activity of COX-3 protein relative to a control cell or population of cells not exposed to said candidate autophagy modulating agent is indicative that said candidate autophagy modulating agent induces autophagy.

According to aspects illustrated herein, there is provided a method a method of identifying a candidate agent that inhibits encephalomyocarditis viral (EMCV) replication comprising: a) providing a composition comprising a COX-3 polypeptide and a candidate agent; (b) determining whether the candidate agent inhibits the COX-3 polypeptide; wherein if the candidate agent inhibits the COX-3 polypeptide, the candidate agent is identified as a candidate agent that inhibits EMCV replication.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10^{th} ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, World Wide Web URL: http://www.ncbi.nlm.nih.gov/omim/ and in Online Mendelian Inheritance in Animals (OMIA), a database of genes, inherited disorders and traits in animal species (other than human and mouse), at http://omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### BRIEF DESCRIPTION OF THE DRAWINGS

FIGS. 1A, 1B and 1C demonstrate analysis of COX-3 mRNA. FIG. 1A depicts a representative polysome profile. RNA from rat spleen was centrifuged through a sucrose gradient and fractionated to separate mRNAs based on polysome density. Fractions were collected using an ISC0-5A spectrophotometer/fractionator while monitoring the absorbance at 254 nm. FIG. 1B shows RT-PCR results for COX-3 and control mRNAs from polysome gradient fractions. FABP, H2A, and PNP-14 all have short open reading frames (of 399, 393, and 405 nucleotides) and are translated by relatively few ribosomes whereas COX-1 and GAPDH with longer reading frames are translated with significantly more ribosomes. Some of the COX-3 mRNA is translated on monosomes while the rest is translated similar to COX-1/GAPDH. FIG. 1C depicts a 5′RACE analysis identifying the transcriptional start sites for rat COX-1 and COX-3. COX-3 utilizes a distinct 5′ cap site six nucleotides downstream from the classical +0 COX-1 cap site. Two additional COX-1 cap sites were identified in our studies, at 28 and at +11. Out of 13 COX-1 clones sequenced, nine used the +11 site, three the +0 site and one the −28 site.

FIGS. 2A, 2B and 2C demonstrate COX-3 expression construct construction. FIG. 2A shows the entire COX-3 transcript including 5′ and 3′ untranslated regions was cloned into the mammalian expression plasmid pcDNA3.1. The splice acceptor site of intron-1 was mutated (AGGA to AAGA) to prevent splicing of the intron upon expression. In some expression constructs, enhanced green fluorescent protein was inserted into the +0 reading frame near the end of the coding sequence between amino acids 500 and 501. Cleavable Flag and 6×His tags were placed at the (−terminus of COX-3 in the +0 reading frame. FIG. 2B depicts site-directed mutagenesis used to mutate the proximal polyadenylation signal sequence, AATAAA, at position 2932 bp to AATCCC to prevent recognition and ensure that the expressed COX-3 mRNA contains the full-length long 3′UTR. The final COX-3 clone was expressed in CHO cells, and analyzed by 3′RACE to confirm that mutation of the polyadenlation site ensures inclusion of the long 3′ untranslated region in the expressed COX-3 message. FIG. 2C shows that mutation of the 3′ splice acceptor site of intron-1 from AGGA to AAGA prevented splicing of the intron following expression in Chinese hamster ovary cells as confirmed by RT-PCR.

FIG. 3 demonstrates fluorescence confocal microscopy of CHO cells transiently expressing COX-3-GFP (green) fusion clone. Cells were co-stained with the nuclear stain TOPRO (blue) and imaged using a laser scanning confocal microscope at 60× magnification. Image is representative of more than 3 experiments.

FIGS. 4A and 4B demonstrate ectopic expression of COX-3 eDNA. FIG. 4A shows an anti-Flag immunoblot of CHO cells transiently expressing either a rat COX-3, COX-1 or empty pcDNA 3.1 expression clone. Five specific bands of indicated molecular weights were seen in COX-3 expressing cells. FIG. 4B depicts results of N-glycanase treatment of COX-3 proteins, demonstrating that only one, the 72 kD form denoted by an asterisk, is N-glycosylated as indicated by an increase in the electrophoretic mobility. The four lower molecular weight COX-3 encoded proteins are not are not affected indicating that they are not glycosylated.

FIGS. 5A and 5B demonstrate stable expression of COX-3. FIG. 5A depicts results of anti-Flag IP of CHO cells stably expressing COX-3 and COX-1. CHO cells were stably transfected with either empty pcDNA3.1 vector, COX-3 or COX-1. Single colonies were isolated and screened for COX expression by anti-flag IP and immunoblot. Colony #2 expresses only the 57, 50 and 44 kD COX-3 forms whereas colony #17 expresses predominantly the 72 kD glycosylated COX-3 form in addition to the lower three forms. COX-1 positive colonies only expressed the 72 kD full-length COX-1 form as well as lower molecular weight breakdown products as demonstrated by tunicamycin treatment, FIG. 5B is a graph illustrating PGE2 synthesis by stable transfectants. Empty Vector, COX-3 and COX-1 colonies were tested for cyclooxygenase activity by anti-PGE2 radioimmunoassay. COX-3 colony #17 had 2 times more activity than the background level measured in cell expressing the empty vector control. Data is a meta-analysis of 3 experiments. * indicates P<0.01.

FIGS. 6A, 6B, 6C and 6D demonstrate site directed mutagenesis of 72 kD COX-3 form. Site-directed mutagenesis was used to insert TAA stop codons into the +0 (FIG. 6A) and +1 (FIG. 6B) reading frames of COX-3 at the indicated nucleotide positions. Additionally site-directed mutagenesis was used to target each codon between 97 and 106 in the +0 reading frame (FIGS. 6C and 6D). Mutated clones were transiently expressed in CHO cells and the level of COX-3 protein expression determined by anti-Flag immunobloting relative to COX-3 control. Stop codon insertional results indicate the presence of two separate initiation sites, one in the +1 reading frame upstream of codon 59, and a second one in the +0 reading frame between codons 94 and 109. Point mutations at positions 47 (ATG to CCC) and 103 (TGC to AAA) confirm that these are the translational start sites for each COX-3 form. Expression measurements were normalized for expression of neomycin phosphotransferase to control for transfection efficiency. Data is the average of 3 or more experiments+/−SEM. *indicates a p-value of <0.01.

FIG. 7 demonstrates AQUA-peptide assisted mass-spectrometry. AQUA-peptides corresponding to the predicted COX-3 protein sequence in both the +1 and +0 reading frames (peptide sequences in bold) were synthesized and analyzed by mass spectrometry. COX-3 cDNA was transiently expressed in CHO cells and encoded proteins purified by Flag IP followed by cobalt resin column. Eluted protein was electrophoresed through a 10% acrylamide gel and stained with COOMASSIE® blue. Proteins with an electrophoretic mobility between 65-80 kD were excised and analyzed by mass spectrometry for the presence of the highlighted peptides (in bold). Peptides in yellow were positively identified while peptides in red were not observed indicating the presence of a third 72 kD COX-3 form which begins translation in the +1 frame then frameshifts into the +0 reading frame at some point in the last 19 amino acids of the +1 open reading frame.

FIGS. 8A and 8B demonstrate site directed mutagenesis of 68 kD COX-3 form. Site-directed mutagenesis was used to (FIG. 8A) introduce TAA stop codons into the +0 reading frame or (FIG. 8B) to mutate codons 250 to 283. These mutated clones were transiently expressed in CHO cells and protein expression determined by anti-Flag immunoblotting. The expression level of the 68 kD COX-3 protein was determined relative to non-mutated COX-3. These results indicate that translation initiates after codon 262 and is dependent upon the region from 256-286 bp. The bar graphs indicate expression of the 68 kd protein relative to the intensity for the un-mutated control. Data is the average of 3 or more experiments+/−SEM. *indicates a p-value of <0.01.

FIGS. 9A, 9B and 9C demonstrate site directed mutagenesis of 57, 50, and 44 kD COX-3 forms. FIG. 9A depicts results of site-directed mutagenesis used to mutate three in-frame ATG codons at positions 487 bp, 673 bp, and 796 bp to GCG (ala) codons. Mutation of each prevented translation of the 57 kD, 50 kD, and 44 kD COX-3 proteins, respectively, indicating that each of these is translated through internal ribosomal initiation at these specific downstream codons. FIG. 9B shows results of 5′ RACE analysis of CHO cells expressing COX-3. 5′RACE analysis demonstrated an absence of cryptic initiation, alternative spicing, or truncated broken mRNAs in the ectopically expressed COX-3 mRNA, eliminating these as a source of the lower molecular weight COX-3 encoded proteins. FIG. 9C is a sequence alignment showing conservation of downstream in-frame ATG codons. Comparison of COX sequences from a selection of vertebrate species demonstrates that all three of the internal initiation sites used by COX-3 are highly conserved. The 57 and 50 kD initiation sites are also conserved in COX-2.

FIGS. 10A, 10B, 10C and 10D demonstrate that 72 kD COX-3 proteins are catalytically active prostaglandin synthase enzymes. FIG. 10A shows that clones were prepared to make predominantly the COX-3 frameshifted 72 kD form (by correcting the frameshift at position 76) and the COX-3 cysteine initiated 72 kD form (by mutating the TGC codon to ATG) and each was expressed in CHO cells. Cyclooxygenase activity was measured in whole cells by adding exogenous arachidonic acid (30 μM) and measuring PGE2 produced by RIA. FIGS. 10B, 10C, and 10D are graphs illustrating the sensitivity of each 72 kD COX-3 protein to SC-560, indomethacin, and acetaminophen was determined and found to be very similar to that of COX-1.

FIG. 11 demonstrates that signal peptide is cleaved from both 72 kD COX-3 forms. His-tags were inserted at the indicated locations in the artificially frameshifted and cysteine initiated forms of COX-3 as well as in COX-1 and transiently expressed in CHO cells. Proteins were purified over a cobalt column and bound protein analyzed by Western blot. Blots were probed using an anti-COX-1 antibody (Cayman). The signal peptide is cleaved off of both the frame-shifted COX-3 form and COX-1 at a point between +210 and +225 (from the COX-3 cap site) corresponding to the reported cleavage site for COX-1. The majority of the cysteine-initiated form of COX-3 is cleaved further downstream at a point after +225 but before +230.

FIGS. 12A, 12B and 12C demonstrate expression of human COX-3. FIG. 12A depicts that human COX-3 was cloned and the eDNA modified to express an authentic COX-3 transcript in the same manner as rat COX-3. This clone was transiently expressed in CHO, A549, 293T, and HeLa cells. As was seen for rat COX-3, lower molecular weight forms were translated in all cell types. However, full-length ˜72 kD COX-3 forms were only detected from CHO and A549 cells. FIG. 12B shows human COX-3 and COX-1 were stably transfected into A549 cells were stably transfected with human COX-3, COX-1, or empty pcDNA 3.1 vector. Single colonies were isolated and analyzed for COX expression by anti-flag immunoblot. FIG. 12C illustrates that point mutation of downstream in-frame ATG codons 557 and 563, 713, 815, and 872 prevented translation of each of the four lower molecular weight proteins. These ATG's are homologous to the ATG codons identified in rat indicating that these act as downstream translation initiation sites in human as well.

FIG. 13 demonstrates a screen of rat tissues for expression of putative COX-3 proteins. Multiple tissues were harvested and analyzed by anti-COX-1 immunoblot for expression of lower molecular weight COX-3 forms. Each tissue was probed with either a COX-1 antibody (+) or a non-immune rabbit control antibody(−) alongside COX-3 size controls. *indicates a COX-1 specific band of the correct size for a COX-3 lower molecular weight form. Results representative of 3 separate experiments.

FIGS. 14A, 14B and 14C demonstrate COX-3 induction in Caco-2 cells. FIG. 14A shows results of RT-PCR analysis of Caco-2 cells treated for 22 hours with 100 mM NaCl (hypertonic conditions) to induce both COX-1 and COX-3 mRNA. FIG. 14B depicts an anti-COX-1 immunoblot of the same salt treated caco-2 cells showing induction of 74 kD, 70 kD, and 54 kD immunoreactive proteins. FIG. 14C depicts results of a Meta-analysis of three experiments measuring the intensity of the 74, 70, and 54 kD forms +/−SEM. *indicates P value of less than 0.05.

FIGS. 15A and 15B demonstrate analysis of human cancer cell lines for expression of COX-3. FIG. 15A illustrates that multiple cancer cells lines were screened by RT-PCR for expression of COX-3 and COX-1 mRNA. FIG. 15B shows that the same cells were screened by immunoblot with either an anti-COX-1 antibody or a non-immune rabbit antibody. Screen demonstrates that the 70 kD and 54 kD COX-3 forms are widely expressed in human cells and that their expression generally correlates with expression of the COX-3 mRNA.

FIGS. 16A, 16B and 16C demonstrate that siRNA knocks-down expression of COX-3 proteins in K562 cells. FIG. 16A shows results of RT-PCR analysis of K562 cells transfected with siRNA against exons 10 and 11 (COX-1/COX-3), intron-1 region of COX-3 (COX-3 specific) or a non-targeting control siRNA. COX-3 is knocked down using siRNAs against exons 10 and 11, but less efficiently using intron-1 specific siRNAs. FIG. 16B shows an immunoblot of the same cells treated with siRNA using an anti-COX-1 antibody detects a decrease in the level of both 74 kD and 70 kD proteins showing that both proteins contain sequence encoded by exons 10 and 11-common to both COX-1 and COX-3 transcripts. siRNA against intron-1 had no effect on the level of 74 kD protein, but significantly decreased the level of the 70 kD protein. FIG. 16C shows results of meta-analysis of three experiments measuring the intensity of the 74 kD and 70 kD immunoblot bands+/−SEM. *indicates a P-value of less than 0.05.

FIGS. 17A, 17B and 17C demonstrate that COX-3 specific siRNA knocks-down expression of 70 kD and 54 kD proteins in MEG-01 cells. FIG. 17A shows results of RT-PCR analysis of MEG-01 cells transfected with siRNAs with either a non-targeting control siRNA, siRNAs targeting multiple regions of exons 10 and 11 (COX-1/COX-3) or siRNAs targeting intron-1 (COX-3 specific). The COX-3 specific siRNAs are not as efficient at knocking down COX-3 siRNA as the COX-1/COX-3 siRNA. FIG. 17B shows an immunoblot of the same siRNA treated cells. These studies indicate that an anti-COX-1 antibody detects a statistically significant decrease in the levels of 74 kD, 70 kD, and 54 kD proteins. Treatment with the COX-3 specific siRNAs indicated 70 kD and 54 kD proteins are derived from the COX-3 mRNA. FIG. 17C shows results of meta-analysis of three experiments measuring the intensity of the 74 kD, 70 kD, and 54 kD immunoblot bands+/−SEM. * indicates a P-value of less than 0.02.

FIG. 18 demonstrates that a confocal image of transiently transfected Myc tagged Nuc into CHO cells is found in a punctate pattern with some localized to ACBD marker while Myc tagged cNuc pattern is mostly cytosolic.

FIG. 19 is a confocal image demonstrating that Myc tagged Nuc is not only found in golgi (as reported before) and that Nuc localized with the autophagosome marker LC3B while cNuc localized with LC3B around large vesicles termed mega-autophagossomes.

FIG. 20 is a confocal image demonstrating that FLAG tagged r57 and r50 transiently transfected into CHO cells exhibits a cytocolic and punctate pattern while r44 has a cytosolic and nuclear pattern.

FIG. 21 is a confocal image demonstrating that FLAG tagged r57 and r50 transiently transfected into CHO cells overlap specific portions of the golgi and both overlap with autophagosome in the periphery of the cell.

FIG. 22 is a confocal image demonstrating FLAG tagged rCOXs or Myc tagged cNuc transiently co-transfected into CHO cells with C-terminal RFP labeled ATG9. cNuc has very little overlap with ATG9-RFP while r57, r50 and r44 highly co-localized with ATG9-RFP while there is little overlap ATG9-RFP when r44 is in the cytosol.

FIG. 23 shows confocal images demonstrating cells transiently co-transfected with cNuc and r57, r50, or r44 and probed against FLAG tagged rCOXs, Myc tagged cNuc, and mannosidase-II. Here r57 and r50 translocate from golgi to co-localize with cNuc around mega-autophagosomes. Note mega-autophagosomes are situated in the periphery when cNuc is co-transfected with r57 or r50 while mega-autophagosome is adjacent to nucleus and r44 co-transfected cells.

FIG. 24 shows confocal images of cells transiently co-transfected with cNuc and r57, r50, or r44 and probed against FLAG tagged rCOXs, Myc tagged cNuc, and autophagosome marker LC3B.

FIG. 25 Is a graph illustrating rCOXs aid in cNuc localization to autophagosome marker LC3B. n=4-9 cells. *=p-value <0.05.

FIG. 26 shows confocal images of cells transiently co-transfected with cNuc and r57, r50, or r44 with the proximal histidine ligand (His388) mutated to glutamine and probed against FLAG tagged rCOXs and Myc tagged cNuc. Here we see rCOXs continue to co-localize with cNuc but we note a large reduction of transiently transfected cells having mega-autophagosome.

FIG. 27 shows confocal images of cells transiently co-transfected with cNuc and r57, r50, or r44 with the distal histidine ligand (His207) mutated to glutamine and probed against FLAG tagged rCOXs, Myc tagged cNuc, and autophagosome marker LC3B. Here we see rCOXs continue to co-localize with cNuc around mega-autophagosomes similar to wild type rCOXs.

FIG. 28 shows confocal images of cells transiently co-transfected with cNuc and r57, r50, or r44 with both distal (His207) and proximal {His388) histidine ligands mutated to glutamine and probed against FLAG tagged rCOXs, Myc tagged cNuc, and autophagosome marker LC3B. We observe rCOXs co-localize very little with cNuc. We also observe cNuc in the periphery of the cell and r57 and r50 found near or around the nucleus.

FIG. 29 shows confocal images of cells transiently co-transfected with cNuc and r57, r50, or r44 with Tyr385, which is important for cyclooxygenase activity, mutated to phenylalanine and probed against FLAG tagged rCOXs and Myc tagged cNuc. We observe r57385Y—7F and r57385Y—7F display similar punctate pattern seen with mutation of proximal and distal histidine ligand constructs. Also, the r44 remains cytosolic with some instances of intranuclear localization and co-localized with cNuc

FIG. 30 is a graph showing the percentage (%) of transiently transfected cells that contain a mega-autophagosome. Here we note an increase in cells with mega-autophagosomes when cNuc is co-transfected with rCOXs. This induction of mega-autophagosome is blocked when the distal (His207) and proximal (His388) histidine ligand are mutated to glutamine. When the Tyr385 or proximal (His388) histidine are mutated we find a dominant negative effect on the percent of transfected cells with mega-autophagosomes. Mutation of the distal (His207) histidine ligand had similar results to wild-type rCOXs. Data represent the average of 3 experiments where 150 to 200 cells were counted in each experiment *=p-value <0.05.

FIG. 31 demonstrates that accumulation of SQST1 (p62) is an indication that autophagic flux has been disrupted. We see no accumulation of p62 in Empty, r57, r50, or r44 while we see p62 accumulation in cNuc and cNuc co-transfected with r57, r50, or r44.

FIG. 32 is a diagram showing autophagy and the markers used to identify the different states of the autophagy.

FIG. 33 shows images of CHO cells co-transfected with Myc tagged cNuc and FLAG tagged rCOXs are probed for Myc, LC3B, and the autolysosome marker cathepsin-D. The images show that mega-autophagosomes do not contain cathepsin-D and would indicate that autophagic flux is blocked before autolysosome formation.

FIG. 34 shows confocal microscopy of cells transiently transfected with Myc tagged cNuc co- and FLAG tagged rCOXs and we probed for Myc, FLAG, and the amphisome marker LAMP-1. The images show that mega-autophagosomes do not contain LAMP-1 and would indicate that autophagic flux is blocked before amphisome formation.

FIGS. 35 A-B show confocal microscopy localization pattern of rc57 and rc50. The images show redox state of rcCOXs modulates their localization pattern A) ATG9 continues to co-localize with rc57 and rc50 with mutation of Tyr385 to a Phe; B) Mutating Tyr385 to a Phe blocked r57 and r50 localization with cNuc and blocked mega-autophagosome formation.

FIGS. 36A-B show confocal microscopy images illustrating the localization pattern of rc57 and rc50. The images show A) ATG9 continues to co-localize with rc57 and rc50 with mutation of H207/388 to a Gin; B) Mutating H207/388 to a Gin blocked r57 and r50 localization with cNuc and blocked mega-autophagosome formation.

FIG. 37 shows a graph depicting oxygen levels in solvent where rc57 was added to the reaction vessel at the time indicated by a downward arrow and linolenic substrate or vehicle was added at the time indicated by an upward arrow. Linolenic acid addition caused a marked decrease in solvent O₂ shown by the displacement of the red line (with linolenic acid) versus the black line (with vehicle). Numbering on graph indicates O₂ consumption per second at varying time frames.

FIG. 38 shows western blot analysis of human COX-3 transcripts wherein downstream ATGs were mutated that blocks the synthesis of recoded proteins similarly found in rat.

FIG. 39 shows confocal microscopy images showing human rc57 co-localizes with human cytosolic nucleobindin at autophagosomes similar to rat rc57.

### DETAILED DESCRIPTION OF THE INVENTION

As described herein, a variety of methods, agents, kits, and compositions are envisioned relating to a method for modulating autophagy in a cell by contacting one or more cells that express COX-3 with an effective amount of an agent that modulates the expression level and/or enzymatic activity of COX-3 or a component thereof. Components may include, but are not limited to, r68, r57, r50 or r44. In describing these methods, agents, kits, and compositions, certain terms and phrases are used throughout as follows.

### I. DEFINITIONS

As used herein, the term “autophagy” refers to a catabolic process involving the degradation of a cell's own components through the lysosomal machinery and is a tightly-regulated process that plays a normal part in cell growth, development, and homeostasis, helping to maintain a balance between the synthesis, degradation, and subsequent recycling of cellular products.

The term “antibody” encompasses immunoglobulins and derivatives thereof containing an immunoglobulin domain capable of binding to an antigen. An antibody can originate from a mammalian or avian species, e.g., human, rodent (e.g., mouse, rabbit), goat, chicken, etc., or can be generated ex vivo using a technique such as phage display. Antibodies include members of the various immunoglobulin classes, e.g., IgG, IgM, IgA, IgD, IgE, or subclasses thereof such as IgG1, IgG2, etc. In various embodiments of the invention “antibody” refers to an antibody fragment or molecule such as an Fab′, F(ab′)2, scFv (single-chain variable) that retains an antigen binding site and encompasses recombinant molecules comprising one or more variable domains (VH or VL). An antibody can be monovalent, bivalent or multivalent in various embodiments. The antibody may be a chimeric or “humanized” antibody. An antibody may be polyclonal or monoclonal, though monoclonal antibodies may be preferred. In some aspects, an antibody is an intrabody, which may be expressed intracellularly.

An “effective amount” or “effective dose” of a compound or other agent (or composition containing such compound or agent) refers to the amount sufficient to achieve a desired biological and/or pharmacological effect, e.g., when delivered to a cell or organism according to a selected administration form, route, and/or schedule. As will be appreciated by those of ordinary skill in this art, the absolute amount of a particular compound, agent, or composition that is effective may vary depending on such factors as the desired biological or pharmacological endpoint, the agent to be delivered, the target tissue, etc. Those of ordinary skill in the art will further understand that an “effective amount” may be contacted with cells or administered in a single dose, or the desired effect may be achieved by use of multiple doses. An effective amount of a composition may be an amount sufficient to reduce the severity of or prevent one or more symptoms or signs of a disorder.

“Contacting”, “contacting the cell” and any derivations thereof as used herein, refers to any means of introducing an agent (e.g., nucleic acids, oligopeptides, ribozymes, antibodies, small molecules, etc) into a target cell, including chemical and physical means, whether directly or indirectly or whether the agent physically contacts the cell directly or is introduced into an environment in which the cell is present. Contacting also is intended to encompass methods of exposing a cell, delivering to a cell, or ‘loading’ a cell with an agent by viral or non-viral vectors, and wherein such agent is bioactive upon delivery. The method of delivery will be chosen for the particular agent and use (e.g., cancer being treated). Parameters that affect delivery, as is known in the medical art, can include, inter alia, the cell type affected (e.g. tumor), and cellular location. In some embodiments, contacting includes administering the agent to a subject. In some embodiments, contacting refers to exposing a cell or an environment in which the cell is located to one or more COX-3 modulating agents of the present invention.

“EMCV” or “encephalomyocarditis virus” is a cardiovirus within the Picornaviridae family. EMCV behaves as an enterovirus in rats, the most common carriers of the virus, as EMCV persists in the gut of these animals for extended periods of time. (Acland, H. and Littlejohns, I.; “Encephalomyocarditis”, Diseases of Swine, editors, Leman, A., et al, The Iowa State University Press, 1981, page 339-343). The host range is very broad and includes primates, mice, elephants, squirrels and swine. Populations of swine, particularly young pigs, are extremely susceptible to EMCV. The virus causes a variety of disease syndromes, including reproductive losses resulting from stillborn, mummified or weak pigs at farrowing. (Links, I., et a., (1986) Aust. Vetern. J. 63:150-151). When suckling or young feeder pigs are infected by the virus, mortality may occur as the result of clinical encephalitis, myocarditis or pneumonia. (Link, supra, and Littlejohn, 1., (1984) Aust. Vetern. J. 61:93). Clinically ill pigs that do not die become inefficient feeders, resulting in performance losses in fattening pigs. Currently, there is no known treatment for an EMCV infection in swine and prevention appears to be limited to the control of rodents on pig farms. The instant invention provides a more effective and efficient means of preventing an EMCV infection in mammals including, e.g., humans, non-human primates, rodents (e.g., mouse, rat, rabbit), ungulates (e.g., ovine, bovine, equine, caprine species), canines, and felines.

“Identity” or “percent identity” is a measure of the extent to which the sequence of two or more nucleic acids or polypeptides is the same. The percent identity between a sequence of interest A and a second sequence B may be computed by aligning the sequences, allowing the introduction of gaps to maximize identity, determining the number of residues (nucleotides or amino acids) that are opposite an identical residue, dividing by the minimum of TG_{A} and TG_{B} (here TG_{A} and TG_{B} are the sum of the number of residues and internal gap positions in sequences A and B in the alignment), and multiplying by 100. When computing the number of identical residues needed to achieve a particular percent identity, fractions are to be rounded to the nearest whole number. Sequences can be aligned with the use of a variety of computer programs known in the art. For example, computer programs such as BLAST2, BLASTN, BLASTP, Gapped BLAST, etc., generate alignments. The algorithm of Karlin and Altschul (Karlin and Altschul, Proc. Natl. Acad. Sci. USA 87:22264-2268, 1990) modified as in Karlin and Altschul, Proc. Natl. Acad Sci. USA 90:5873-5877, 1993 is incorporated into the NBLAST and XBLAST programs of Altschul et al. (Altschul, et al., J. Mol. Biol. 215:403-410, 1990). In some embodiments, to obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. See the Web site having URL www.ncbi.nlm.nih.gov. Other suitable programs include CLUSTALW (Thompson J D, Higgins D G, Gibson T J, Nuc Ac Res, 22:4673-4680, 1994) and GAP (GCG Version 9.1; which implements the Needleman & Wunsch, 1970 algorithm (Needleman S B, Wunsch C D, J Mol Biol, 48:443-453, 1970.)

“Isolated” refers to a substance that is separated from at least some other substances with which it is normally found in nature, usually by a process involving the hand of man, or is artificially produced, e.g., chemically synthesized, or present in an artificial environment. In some embodiments, any of the nucleic acids, polypeptides, nucleic-acid-protein structures, protein complexes, or cells of the invention, is isolated. In some embodiments, an isolated nucleic acid is a nucleic acid that has been synthesized using recombinant nucleic acid techniques or in vitro transcription or chemical synthesis or PCR. In some embodiments, an isolated polypeptide is a polypeptide that has been synthesized using recombinant nucleic acid techniques or in vitro translation or chemical synthesis.

“Modulation” and “modulating” are used interchangeably to refer to a perturbation of function or activity when compared to the level of the function or activity prior to modulation. For example, in the context of gene expression, modulation includes the change, either an increase (stimulation or induction) or a decrease (inhibition or reduction) in gene expression. In the context of autophagy, modulation includes the change, either an increase (stimulation or induction) in autophagy, which is useful, e.g., to inhibit the growth or proliferation of tumor cells or suppress viral infections, or a decrease (inhibition or reduction) in autophagy, which is useful, e.g., to inhibit viral replication.

“Nucleic acid” is used interchangeably with “polynucleotide” and encompasses naturally occurring polymers of nucleosides, such as DNA and RNA, usually linked by phosphodiester bonds, and non-naturally occurring polymers of nucleosides or nucleoside analogs. In some embodiments a nucleic acid comprises standard nucleotides (abbreviated A, G, C, T, U). In other embodiments a nucleic acid comprises one or more non-standard nucleotides. In some embodiments, one or more nucleotides are non-naturally occurring nucleotides or nucleotide analogs. A nucleic acid can be single-stranded or double-stranded in various embodiments of the invention. A nucleic acid can comprise chemically or biologically modified bases (for example, methylated bases), modified sugars (2′-fluororibose, arabinose, or hexose), modified phosphate groups (for example, phosphorothioates or 5′-N-phosphoramidite linkages), locked nucleic acids, or morpholinos. In some embodiments, a nucleic acid comprises nucleosides that are linked by phosphodiester bonds. In some embodiments, at least some nucleosides are linked by a non-phosphodiester bond. A nucleic acid can be single-stranded, double-stranded, or partially double-stranded. An at least partially double-stranded nucleic acid can have one or more overhangs, e.g., 5′ and/or 3′ overhang(s). Nucleic acid modifications (e.g., nucleoside and/or backbone modifications), non-standard nucleotides, delivery vehicles and approaches, etc., known in the art as being useful in the context of RNA interference (RNAi), aptamer, or antisense-based molecules for research or therapeutic purposes are contemplated for use in various embodiments of the instant invention. See, e.g., Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008. A nucleic acid may comprise a detectable label, e.g., a fluorescent dye, radioactive atom, etc. “Oligonucleotide” refers to a relatively short nucleic acid, e.g., typically between about 4 and about 60 nucleotides long. The terms “polynucleotide sequence” or “nucleic acid sequence” as used herein can refer to the nucleic acid material itself and is not restricted to the sequence information (i.e. the succession of letters chosen among the five base letters A, G, C, T, or U) that biochemically characterizes a specific nucleic acid, e.g., a DNA or RNA molecule. A naturally occurring nucleic acid or a nucleic acid identical in sequence to a naturally occurring nucleic acid may be referred to herein as a “native nucleic acid”, a “native XXX nucleic” (where XXX represents the name of the nucleic acid), or simply by the name of the nucleic acid or gene.

A “polypeptide” refers to a polymer of amino acids linked by peptide bonds. A protein is a molecule comprising one or more polypeptides. A peptide is a relatively short polypeptide, typically between about 2 and 60 amino acids in length. The terms “protein”, “polypeptide”, and “peptide” may be used interchangeably.

A “multisubunit protein” is composed of multiple polypeptide chains physically associated with one another to form a complex. Polypeptides of interest herein often contain standard amino acids (the 20 L-amino acids that are most commonly found in nature in proteins). However, other amino acids and/or amino acid analogs known in the art can be used in certain embodiments of the invention. One or more of the amino acids in a polypeptide (e.g., at the N- or C-terminus or in a side chain) may be altered, for example, by addition, e.g., covalent linkage, of a moiety such as an alkyl group, carbohydrate group, a phosphate group, a halogen, a linker for conjugation, etc. A polypeptide sequence presented herein is presented in an N-terminal to C-terminal direction unless otherwise indicated. “Polypeptide domain” refers to a segment of amino acids within a longer polypeptide. A polypeptide domain may exhibit one or more discrete binding or functional properties, e.g., a binding activity or a catalytic activity. A domain may be recognizable by its conservation among polypeptides found in multiple different species. The term “polypeptide sequence” or “amino acid sequence” as used herein can refer to the polypeptide material itself and is not restricted to the sequence information (i.e. the succession of letters or three letter codes chosen among the letters and codes used as abbreviations for amino acid names) that biochemically characterizes a polypeptide. A naturally occurring polypeptide or a polypeptide identical in sequence to a naturally occurring polypeptide may be referred to herein as a “native polypeptide”, a “native XXX polypeptide” (where XXX represents the name of the polypeptide), or simply by the name of the polypeptide.

A “variant” of a nucleic acid refers to a nucleic acid that differs by one or more nucleotide substitutions, additions, or deletions, relative to a native nucleic acid. An addition can be an insertion within the nucleic acid or an addition at the 5′- or 3′-terminus. A deletion can be a deletion of a 5′-terminal region, 3′-terminal region and/or an internal region. A “variant” of a polypeptide refers to a polypeptide that differs by one or more nucleotide amino acid substitutions, additions, or deletions, relative to a native polypeptide. An addition can be an insertion within the polypeptide or an addition at the N- or C-terminus. A deletion can be a deletion of an N-terminal region, a C-terminal region, and/or an internal region. In some embodiments, the number of nucleotides or amino acids substituted in and/or added to a native nucleic acid or polypeptide or portion thereof can be for example, about 1 to 30, e.g., about 1 to 20, e.g., about 1 to 10, e.g., about 1 to 5, e.g., 1, 2, 3, 4, or 5. In some embodiments, the number of nucleotides or amino acids substituted in and/or added to a native nucleic acid or polypeptide or portion thereof can be for example, between 0.1% and 10% of the total number of nucleotides or amino acids in such native nucleic acid or polypeptide or portion thereof. In some embodiments, a variant comprises a nucleic acid or polypeptide whose sequence is at least 20%, 30%, 40%, 50%, 60%, 70%, 80%, 85%, 90%, 95%, 96%, 97%, 98%, 99%, 99.5%, or more identical in sequence to a native nucleic acid polypeptide (e.g., from a vertebrate such as a human, mouse, rat, cow, or chicken) over at least 50, 100, 150, 200, 250, 300, 400, 450, or 500 amino acids (but is not identical in sequence to native nucleic acid or polypeptide). In some embodiments, a variant comprises a nucleic acid or polypeptide at least 20%, 30%, 40%, 50%, 60%, 70%, 80%, 85%, 90%, 95%, 96%, 97%, 98%, 99%, 99.5%, or more identical in sequence to a native nucleic acid or polypeptide (e.g., from a vertebrate such as a human, mouse, rat, cow, or chicken) over at least 20%, 30%, 40%, 50%, 60%, 70%, 80%, 85%, 90%, 95%, 96%, 97%, 98%, 99%, 99.5%, or 100% of the native nucleic acid or polypeptide. In some embodiments, a variant nucleic acid or polypeptide comprises or consists of a fragment. A fragment is a nucleic acid or polypeptide that is shorter than a particular nucleic acid polypeptide and is identical in sequence to the nucleic acid polypeptide over the length of the shorter nucleic acid or polypeptide. In some embodiments, a fragment is at least 50%, 60%, 70%, 80%, 85%, 90%, 95%, 96%, 97%, 98%, or 99% as long as a native nucleic acid or polypeptide.

In some embodiments, a polypeptide fragment is an N-terminal fragment (i.e., it lacks a C-terminal portion of the native polypeptide). In some embodiments, a fragment is a C-terminal fragment (i.e., it lacks an N-terminal portion of the native polypeptide). In some embodiments, a fragment is an internal fragment, i.e., it lacks an N-terminal portion and a C-terminal portion of the native polypeptide. In some embodiments, a variant comprises two fragments fused together, e.g., an N-terminal portion and a C-terminal portion.

In some embodiments, a variant polypeptide comprises a heterologous polypeptide portion. The heterologous portion often has a sequence that is not present in the native polypeptide. In some embodiments, a heterologous portion has a sequence that is present in the native polypeptide, but at a different position. For example, a domain can be duplicated or positioned at a different location within the polypeptide. A heterologous polypeptide portion may be, e.g., between 5 and about 5,000 amino acids long, or longer, respectively, in various embodiments. Often it is between 5 and about 1,000 amino acids long. In some embodiments, a heterologous portion comprises a sequence that is found in a different polypeptide, e.g., a functional domain. In some embodiments, a heterologous portion comprises a sequence useful for purifying, expressing, solubilizing, and/or detecting the polypeptide. In some embodiments, a heterologous portion comprises a polypeptide “tag”, e.g., an affinity tag or epitope tag. For example, the tag can be an affinity tag (e.g., HA, TAP, Myc, 6×His, Flag, GST), solubility-enhancing tag (e.g., a SUMO tag, NUS A tag, SNUT tag, or a monomeric mutant of the Ocr protein of bacteriophage T7). See, e.g., Esposito D and Chatterjee D K. Curr Opin Biotechnol.; 17(4):353-8 (2006). In some embodiments, a tag can serve multiple functions. A tag is often relatively small, e.g., ranging from a few amino acids up to about 100 amino acids long. In some embodiments a tag is more than 100 amino acids long, e.g., up to about 500 amino acids long, or more. In some embodiments, a variant has a tag located at the N- or C-terminus, e.g., as an N- or C-terminal fusion. The polypeptide could comprise multiple tags. In some embodiments, a 6×His tag and a NUS tag are present, e.g., at the N-terminus. In some embodiments, a tag is cleavable, so that it can be removed from the polypeptide, e.g., by a protease. Exemplary proteases include, e.g., thrombin, TEV protease, Factor Xa, PreScission protease, etc. In some embodiments, a “self-cleaving” tag is used. See, e.g., PCT/US05/05763. Sequences encoding a tag can be located 5′ or 3′ with respect to a polynucleotide encoding the polypeptide (or both). In some embodiments, a heterologous portion comprises a detectable marker such as a fluorescent or luminescent protein, e.g., green, blue, sapphire, yellow, red, orange, and cyan fluorescent protein or derivatives thereof (e.g., EGFP, ECFP, EYFP), or monomeric red fluorescent protein or derivatives such as those known as “mFruits”, e.g., mCherry, mStrawberry, mTomato, or Cerulean or DsRed. In some embodiments, a heterologous portion comprises an enzyme that catalyzes a reaction leading to a detectable reaction product in the presence of a suitable substrate. Examples include alkaline phosphatase, beta galactosidase, horseradish peroxidase, luciferase, to name a few. Often, a detectable marker or reaction product is optically detectable, emitting or absorbing electromagnetic radiation (e.g., within the visible or near infrared region of the spectrum) that can be observed visually and/or using suitable detection equipment. Detectable markers can include moieties that quench signals emitted from other moieties. In some embodiments, a heterologous portion comprises a selectable marker, e.g., a drug resistance marker or nutritional marker. Exemplary drug resistance markers include enzymes that inactivate compounds that would otherwise be cytotoxic or inhibit cell proliferation (e.g., neomycin or G418 resistance gene, puromycin resistance gene, blastocidin resistance gene etc.). A nutritional marker is typically an enzyme that permits a cell to survive in medium that lacks a particular nutrient. In some embodiments a tag or other heterologous portion is separated from the rest of the polypeptide by a polypeptide linker. For example, a linker can be a short polypeptide (e.g., 15-25 amino acids). Often a linker is composed of small amino acid residues such as serine, glycine, and/or alanine. A heterologous domain could comprise a transmembrane domain, a secretion signal domain, a domain that targets the polypeptide to a particular organelle, etc.

In some embodiments, a variant is a functional variant, i.e., the variant at least in part retains at least one biological activity of a native polypeptide, such as ability to bind to a particular molecule or structure, or ability to catalyze a biochemical reaction (or is a nucleic acid that encodes a functional variant polypeptide). One of skill in the art can readily generate functional variants or fragments. In some embodiments, a variant comprises one or more conservative amino acid substitutions relative to a native polypeptide. Conservative substitutions may be made on the basis of similarity in side chain size, polarity, charge, solubility, hydrophobicity, hydrophilicity and/or the amphipathic nature of the residues involved. As known in the art, such substitutions are, in general, more likely to result in a variant that retains activity as compared with non-conservative substitutions. In one embodiment, amino acids are classified as follows:

### Special: C

Neutral and small: A, G, P, S, T
Polar and relatively small: N, D, Q, E
Polar and relatively large: R, H, K
Nonpolar and relatively small: I, L, M, V
Nonpolar and relatively large: F, W, Y

### Special: C

See, e.g., Zhang, J. J. Mol. Evol. 50:56-68, 2000). In some embodiments, proline (P) is considered to be in its own group as a second special amino acid. Within a particular group, certain substitutions may be of particular interest, e.g., replacements of leucine by isoleucine (or vice versa), serine by threonine (or vice versa), or alanine by glycine (or vice versa). Of course non-conservative substitutions are often compatible with retaining function as well. In some embodiments, a substitution, deletion, or addition does not alter or delete or disrupt an amino acid or region of a polypeptide known or thought to be involved in or required for a particular activity that is desired to be maintained, while in other embodiments a substitution, deletion, or addition is selected to remove or disrupt a region known or thought be to in involved in or required for a particular activity. In some embodiments, an alteration is at an amino acid that differs among homologous polypeptides of different species. Variants could be tested in cell-free and/or cell-based assays to assess their activity.

In some embodiments, a variant or fragment that has substantially reduced activity as compared with the activity of native polypeptide (e.g., less than 10% of the activity of native polypeptide) is useful. For example, such polypeptide could interfere with the function of native polypeptide, e.g., by competing with native polypeptide, or serve as an immunogen for purposes of raising antibodies.

In some embodiments, a variant nucleic acid comprises a heterologous nucleic acid portion, which may be located at the 5′-terminus, 3′-terminus, or internally. The heterologous portion often has a sequence that is not present in the native nucleic acid. In some embodiments, a heterologous portion has a sequence that is present in the native nucleic acid, but at a different position. A heterologous nucleic acid portion may encode a heterologous polypeptide portion, such as any of those described above, or may not encode a polypeptide. A heterologous nucleic acid portion may or may not have a property or activity such as serving as an expression control element, recognition sequence for a DNA binding protein, or encoding a functional RNA.

As used herein, the term “purified” refers to agents or entities (e.g., compounds such as polypeptides, nucleic acids, small molecules, etc.) that have been separated from most of the components with which they are associated in nature or when originally generated. In general, such purification involves action of the hand of man. Purified agents or entities may be partially purified, substantially purified, or pure. Such agents or entities may be, for example, at least 50%, 60%, 70%, 75%, 80%, 85%, 90%, 95%, 96%, 97%, 98%, 99%, or more than 99% pure. In some embodiments, a nucleic acid or polypeptide is purified such that it constitutes at least 75%, 80%, 855%, 90%, 95%, 96%, 97%, 98%, 99%, or more, of the total nucleic acid or polypeptide material, respectively, present in a preparation. Purity can be based on, e.g., dry weight, size of peaks on a chromatography tracing, molecular abundance, intensity of bands on a gel, or intensity of any signal that correlates with molecular abundance, or any art-accepted quantification method. In some embodiments, water, buffers, ions, and/or small molecules (e.g., precursors such as nucleotides or amino acids), can optionally be present in a purified preparation. A purified molecule may be prepared by separating it from other substances (e.g., other cellular materials), or by producing it in such a manner to achieve a desired degree of purity. In some embodiments, a purified molecule or composition refers to a molecule or composition that is prepared using any art-accepted method of purification. In some embodiments “partially purified” means that a molecule produced by a cell is no longer present within the cell, e.g., the cell has been lysed and, optionally, at least some of the cellular material (e.g., cell wall, cell membrane(s), cell organelle(s)) has been removed. In some embodiments, any of the nucleic acids, polypeptides, nucleic-acid-protein structures, or protein complexes of the invention, is at least partly purified.

A “small molecule” as used herein, is an organic molecule that is less than about 2 kilodaltons (KDa) in mass. In some embodiments, the small molecule is less than about 1.5 KDa, or less than about 1 KDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding, e.g., a amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

A “subject” can be any multicellular animal, e.g., a vertebrate, e.g., a mammal or avian. Exemplary mammals include, e.g., humans, non-human primates, rodents (e.g., mouse, rat, rabbit), ungulates (e.g., ovine, bovine, equine, caprine species), canines, and felines. In some embodiments, the animal is a mammal of economic importance, such as a cow, horse, pig, goat, or sheep.

In practicing the many aspects of the invention herein, samples e.g., biological samples can be selected from many sources such as tissue biopsy (including cell sample or cells cultured therefrom; biopsy of bone marrow or solid tissue, for example cells from a solid tumor), blood, blood cells (red blood cells or white blood cells), serum, plasma, lymph, ascetic fluid, cystic fluid, urine, sputum, stool, saliva, bronchial aspirate, CSF or hair. Cells from a sample can be used, or a lysate of a cell sample can be used. In certain embodiments, the biological sample is a tissue biopsy cell sample or cells cultured therefrom, for example, cells removed from a solid tumor or a lysate of the cell sample. In certain embodiments, the biological sample comprises blood cells.

“Treat”, “treating” and similar terms refer to providing medical and/or surgical management of a subject. Treatment can include, but is not limited to, administering a compound or composition (e.g., a pharmaceutical composition or a composition comprising appropriate cells in the case of cell-based therapy) to a subject. Treatment is typically undertaken in an effort to alter the course of a disorder (which term is used to refer to a disease, syndrome, or abnormal condition) or undesirable or harmful condition in a manner beneficial to the subject. The effect of treatment can generally include reversing, alleviating, reducing severity of, delaying the onset of, curing, inhibiting the progression of, and/or reducing the likelihood of occurrence or reoccurrence of the disorder or condition, or one or more symptoms or manifestations of such disorder or condition. A composition can be administered to a subject who has developed a disorder or is at risk of developing a disorder. A composition can be administered prophylactically, i.e., before development of any symptom or manifestation of a disorder. Typically in this case the subject will be at increased risk of developing the disorder relative to a member of the general population. For example, a composition can be administered to a subject with a risk factor, e.g., a mutation in a gene, wherein the risk factor is associated with increased likelihood of developing the disorder but before the subject has developed symptoms or manifestations of the disorder. “Preventing” can refer to administering a composition to a subject who has not developed a disorder, so as to reduce the likelihood that the disorder will occur or so as to reduce the severity of the disorder should it occur. The subject may be identified (e.g., diagnosed by a medical practitioner) as having or being at risk of developing the disorder (e.g., at increased risk relative to many most other members of the population or as having a risk factor that increases likelihood of developing the disorder).

Pharmaceutical compositions for use in the present invention can include compositions comprising one or a combination of COX inhibitors in an effective amount to achieve the intended purpose. The determination of an effective dose of a pharmaceutical composition of the invention is well within the capability of those skilled in the art. A therapeutically effective dose refers to that amount of active ingredient which ameliorates the symptoms or condition. Therapeutic efficacy and toxicity can be determined by standard pharmaceutical procedures in cell cultures or experimental animals, for example the ED50 (the dose therapeutically effective in 50% of the population) and LD50 (the dose lethal to 50% of the population).

As used herein “COX” or “COX protein” refers generally to a family of proteins involved in the synthesis of prostaglandins. More specifically, “COX” includes cyclooxygenase-1 (COX-1), cyclooxygenase-2 (COX-2), and cyclooxygenase-3 (COX-3).

As used herein “COX-3 overexpression”, and “increased level and/or activity of COX-3” is meant to encompass a level and/or activity of COX-3 or COX-3 protein that is at least about 1, 2, 3, 4, 5, 6, 7, 8, 9, 10 fold or more higher than a reference or normal level and/or activity of COX-3 or COX-3 protein. However, modest increased levels and/or activity, such as about 0.1, 0.2, 0.3, 0.4, 0.5, 0.6, 0.7, 0.8, 0.9 fold higher levels and/or activity than a reference or normal level or activity of COX-3 are also encompassed by this phrase.

As used herein “COX-3 modulating agents” refer to modulating agents of COX-3, including agents that inhibit the level and/or activity of COX-3 and/or components of COX-3 e.g., r68, r57, r50, and r44, as well as agents that activate or increase the level and/or activity of COX-3 and/or components of COX-3 e.g., r68, r57, r50, and r44. In some embodiments, COX-3 modulating agents include molecules that bind directly to a functional region of COX-3 in a manner that interferes with the enzymatic activity of COX-3 e.g., agents that interfere with substrate binding to COX-3. COX-3 modulating agents include agents that inhibit the activity of peptides, polypeptides, or proteins that modulate the activity of COX-3 and/or components of COX-3 e.g., inhibitors of r68, r57, r50, and r44, Examples of suitable modulating agents include, but are not limited to antisense oligonucleotides, oligopeptides, interfering RNA e.g., small interfering RNA (siRNA), small hairpin RNA (shRNA), aptamers, ribozymes, small molecule inhibitors, or antibodies or fragments thereof, and combinations thereof. In some embodiments, COX-3 modulating agents are specific inhibitors or specifically inhibit the level and/or activity of COX-3 and or components of COX-3. As used herein, “specific inhibitor(s)” refers to inhibitors characterized by their ability to bind to with high affinity and high specificity to COX-3 proteins or domains, motifs, or fragments thereof, or variants thereof, and preferably have little or no binding affinity for non-COX-3 proteins. As used herein, “specifically inhibit(s)” refers to the ability of a COX-3 modulating agent of the present invention to inhibit the level and/or activity of a target polypeptide e.g., COX-3, and/or r68, r57, r50, and r44, and preferably have little or no inhibitory effect on non-target polypeptides. As used herein, “specifically activate(s)” and “specifically increase(s)” refers to the ability of a COX-3 modulating agent of the present invention to stimulate (e.g., activate or increase) the level and/or activity of a target polypeptide, e.g., COX-3, and/or r68, r57, r50, and r44 and preferably to have little or no stimulatory effect on non-target polypeptides.

As used herein “level”, refers to a measure of the amount of, or a concentration of a transcription product, for instance an mRNA, or a translation product, for instance a protein or polypeptide.

As used herein “activity” refers to a measure for the ability of a transcription product or a translation product to produce a biological effect or a measure for a level of biologically active molecules.

As used herein, enzymatic activity refers to the ability of an enzyme to act as a catalyst in a process, such as the conversion of one compound to another compound.

As used herein “level and/or activity” further refer to gene expression levels or gene activity. Gene expression can be defined as the utilization of the information contained in a gene by transcription and translation leading to the production of a gene product.

As used herein, the term “downregulating COX-3 expression” refers to a substantial reduction e.g., measurable or observable, in the expression of the COX-3 protein or isoform thereof in a target cell through any of the methods disclosed herein or those known to one of ordinary skill in the art, with the benefit of the present disclosure. For example, in some embodiments downregulating COX-3 expression reduces the expression of the COX-3 protein or isoform thereof by at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, or more relative to the expression level of COX-3 protein or isoform thereof in the target cell in the absence of attempting to down-regulate COX-3 expression in the target cell in accordance with the present disclosure. In some embodiments downregulating COX-3 expression reduces the expression of the COX-3 protein or isoform thereof by at least 1.1 fold, at least 1.2 fold, 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, at least 5 fold, at least 10 fold, at least 20 fold, at least 30 fold, at least 40 fold, at least 50 fold, or at least 100 fold, at least a 1,000 fold, at least 10,000 fold, or more relative to the expression level of COX-3 protein or isoform thereof in the target cell in the absence of attempting to down-regulate COX-3 expression in the target cell in accordance with the present disclosure.

As used herein, the term “inhibiting COX-3 translation” refers to a substantial reduction e.g., measurable or observable, in the translation (e.g., amount and frequency) of the COX-3 protein in the target cell from RNA encoding the COX-3 protein, including RNA natively transcribed by the target cell and RNA artificially introduced into the target cell. For example, in some embodiments inhibiting COX-3 translation reduces translation of the COX-3 protein or isoform thereof in the target cell from RNA encoding the COX-3 protein by at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, or more relative to the translation of COX-3 protein or isoform thereof in the target cell in the absence of attempting to inhibit COX-3 translation in the target cell in accordance with the present disclosure. In some embodiments inhibiting COX-3 translation reduces translation of the COX-3 protein or isoform thereof in the target cell from RNA encoding the COX-3 protein by at least 1.1 fold, at least 1.2 fold, 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, at least 5 fold, at least 10 fold, at least 20 fold, at least 30 fold, at least 40 fold, at least 50 fold, or at least 100 fold, at least a 1,000 fold, at least 10,000 fold, or more relative to the translation of COX-3 protein or isoform thereof in the target cell in the absence of attempting to inhibit COX-3 translation in the target cell in accordance with the present disclosure.

As used herein, the term “inhibiting COX-3 enzymatic activity” refers to a substantial reduction e.g., measurable or observable, in the ability of COX-3 to act as an enzyme (i.e. have a designated effect on one or more substrate molecules) through any of the methods disclosed herein or those known to one of ordinary skill in the art, with the benefit of the present disclosure. As used herein, “COX-3 enzymatic activity” refers to any enzymatic activity performed by COX-3 protein or isoform thereof, including without limitation, cyclooxygenase or cyclooxygenase-like activity (e.g., ability to oxygenate lipids, i.e., lipooxygenase activity), and peroxidase or peroxidase-like activity.

For example, in some embodiments inhibiting COX-3 enzymatic activity reduces activity of the COX-3 protein or isoform thereof in the target cell by at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, or more relative to the enzymatic activity of COX-3 protein or isoform thereof in the target cell in the absence of attempting to inhibit COX-3 enzymatic activity in the target cell in accordance with the present disclosure. In some embodiments inhibiting COX-3 enzymatic activity reduces activity of the COX-3 protein or isoform thereof in the target cell by at least 1.1 fold, at least 1.2 fold, 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, at least 5 fold, at least 10 fold, at least 20 fold, at least 30 fold, at least 40 fold, at least 50 fold, or at least 100 fold, at least a 1,000 fold, at least 10,000 fold, or more relative to the enzymatic activity of COX-3 protein or isoform thereof in the target cell in the absence of attempting to inhibit COX-3 enzymatic activity in the target cell in accordance with the present disclosure. In some embodiments, inhibiting COX-3 enzymatic activity completely abolishes enzymatic activity of COX-3 or the isoform thereof.

In some embodiments, the present disclosure provides a method for treating cancer comprising inhibiting COX-3 or an isoform thereof. Without wishing to be bound by theory, it is believed that inhibiting COX-3 or isoform thereof increases autophagy in cells so as to inhibit the growth and/or proliferation of tumor cells, thereby treating cancer. In some embodiments, the inhibition of COX-3 may comprise downregulating COX-3 expression. Suitable methods for downregulating COX-3 expression may include: inhibiting transcription of COX-3 mRNA; degrading COX-3 mRNA by methods including, but not limited to, the use of interfering RNA (RNAi); blocking translation of COX-3 mRNA by methods including, but not limited to, the use of antisense nucleic acids or ribozymes, or the like. In some embodiments, a suitable method for downregulating COX-3 expression may include providing to the cancer a small interfering RNA (siRNA) targeted to COX-3. In some embodiments, such a small molecule may comprise staurosporine. In some embodiments, suitable methods for down-regulating COX-3 may include administering a small molecule inhibitor of COX-3. In some embodiments, it may be advantageous to use two or more of these methods simultaneously or in series. One of ordinary skill in the art, with the benefit of the present disclosure, may recognize suitable methods for downregulating COX-3 expression that are still considered within the scope of the present disclosure.

### II. METHODS FOR TARGETING COX-3

Cyclooxygenase (COX), officially known as prostaglandin-endoperoxide synthase (PTGS), is an enzyme that is responsible for formation of important biological mediators called prostanoids, including prostaglandins, prostacyclin and thromboxane. Pharmacological inhibition of COX can provide relief from the symptoms of inflammation and pain. Non-steroidal anti-inflammatory drugs (NSAID), such as aspirin ibuprofen and naproxen, and paracetamol, phenacetin, antipyrine, dipyrone, for example, exert their effects through inhibition of COX.

The present invention is based, at least in part, on targeting certain molecules, referred to herein as “cyclooxygenase type 1 variants,” “COX-1 variants” or “COX-1 variant nucleic acid and polypeptide molecules,” which play a role in or function in signaling pathways associated with cell processes in brain and other tissues. Exemplary COX-1 variants of the invention include COX-3 or COX-1b. In one embodiment, the COX-1 variant molecules modulate the activity of one or more proteins involved in cellular growth or differentiation. In another embodiment, the COX-1 variant molecules of the present invention are capable of modulating autophagy. Fatty acid oxygenase activity is central to the production of prostaglandins, thromboxanes, hydroxy- and hydroperoxy-fatty acids by cyclooxygenases and is also shared by a related group of enzymes, which in plants are called pathogen inducible fatty acid oxygenases (PIOXs). PIOXs make hydroperoxy-fatty acids and their derivatives. Thus, the present COX-1 variants, like PIOXs, contain the critical amino acid residues needed to synthesize important oxygenated fatty acid-derived messengers in the brain and in other tissue.

As previously noted, cyclooxygenases play a role in prostaglandin synthesis. Inhibition or over stimulation of the activity of cyclooxygenases involved in signaling pathways associated with cellular growth can lead to perturbed cellular growth, which can in turn lead to cellular growth related disorders. As used herein, a “cellular growth related disorder” includes a disorder, disease, or condition characterized by a deregulation, e.g., an upregulation or a downregulation, of cellular growth. Cellular growth deregulation may be due to a deregulation of cellular proliferation, cell cycle progression, cellular differentiation and/or cellular hypertrophy. Examples of cellular growth related disorders include disorders such as cancer, e.g., melanoma, prostate cancer, cervical cancer, breast cancer, colon cancer, or sarcoma, Cellular growth related disorders further include disorders related to unregulated or dysregulated apoptosis (i.e., programmed cell death). Apoptosis is a cellular suicide process in which damaged or harmful cells are eliminated from multicellular organisms. Cells undergoing apoptosis have distinct morphological changes including cell shrinkage, membrane blebbing, chromatin condensation, apoptotic body formation and fragmentation. This cell suicide program is evolutionarily conserved across animal and plant species. Apoptosis plays an important role in the development and homeostasis of metazoans and is also critical in insect embryonic development and metamorphosis. Furthermore, apoptosis acts as a host defense mechanism. For example, virally infected cells are eliminated by apoptosis to limit the propagation of viruses. Apoptosis mechanisms are involved in plant reactions to biotic and abiotic insults. Dysregulation of apoptosis has been associated with a variety of human diseases including cancer, neurodegenerative disorders and autoimmune diseases. Accordingly, identification of novel mechanisms to manipulate apoptosis provides new means to study and manipulate this process.

In some aspects, the invention provides for methods of modulating autophagy, the method including contacting one or more cells that expresses COX-3 with an effective amount of an agent that modulates the expression level and/or enzymatic activity of COX-3 or a isoform thereof. In some embodiments, modulating autophagy includes inhibiting autophagy. In some embodiments, an agent may inhibit autophagy by interfering with the interaction between COX-3 protein or an isoform thereof and nucleobindin (Nuc). By interfering with the interaction between COX-3 protein and Nuc, the agent prevents the formation of mega-autophagosomes. In some embodiments, inhibiting autophagy may inhibit autophagy-associated viral replication. As used herein, “autophagy-associated viral replication” refers to replication that involves, depends on, or avoids autophagy as a means of increasing viral numbers. Examples of disease or conditions that involve autophagy-associated viral replication include, but are not limited to, influenza, adenoviruses, enterovirus, EMCV, ebola virus, rabies, and HCV. See Zhou Z. Autophagy. 2009 April; 5 (3):321-8; Rodriguez-Rocha H Virology. 2011 July 20; 416(1-2):9-15; Lee Y R Journal of Biomedical Science 2014, 21:80; Geisbert T W Am J Pathol. 2003 December; 163(6):2347-70. EMCV replication causes encephalomyocarditis and reproductive disease in mammals including, e.g., humans, non-human primates, rodents (e.g., mouse, rat, rabbit), ungulates (e.g., ovine, bovine, equine, caprine species), canines, and felines. Although a variety of mammals may host the virus, pigs are classed as the domestic host as they are most easily infected.

In some embodiments, modulating autophagy includes inducing autophagy. Inducing autophagy may act to suppress viral infection. Suppressing viral infection can be useful for the treatment of certain viral infections. In some embodiments, an agent may induce autophagy by promoting the interaction between COX-3 protein or an isoform thereof and nucleobindin (Nuc). By promoting the interaction between COX-3 protein and Nuc, the agent promotes the formation of mega-autophagosomes. In some embodiments, inducing autophagy may inhibit or treat autophagy-associated viral infections. As used herein, “autophagy-associated viral infections” refers to infections that involve, depend on, or avoid autophagy as a means of increasing viral numbers. Autophagy-associated viral infections may be caused, in one embodiment, by an RNA virus. Examples of RNA viruses include, but are not limited to, coxsackievirus, poliovirus, vesicular stomatitis virus, human immunodeficiency virus, hepatitis C virus, rubella virus and morbilliviruses. Autophagy-associated viral infections may be caused, in another embodiment, by a DNA virus. Examples of DNA viruses include, but are not limited to vaccinia virus, herpes simplex viruses (HSV-1 and -2), Epstein-Barr virus, hepatitis B virus, parvovirus and varicella zoster.

In some embodiments, suppressing viral infection includes inhibiting and/or eliminating a significant fraction of the viral particles. In some embodiments, a significant fraction includes a majority of the viral particles present in the cell. In some embodiments, a significant fraction comprises at least 1%, 10%, 20%, 30%, 40%, 50%, 60%, 70%, 80%, 85%, 90%, 95%, 96%, 97%, 98%, 99%, 99.5%, or up to about 100% of the viral particles in the cell. In one embodiment, a significant fraction includes up to about 100% of the viral particles in the cell. In one embodiment, a significant fraction includes 100% of the viral particles in the cell.

In other embodiments, modulating autophagy may include inducing autophagy. Inducing autophagy may act to inhibit the growth or proliferation of tumor cells. Inhibiting the proliferation of, and/or eliminating one or more tumor cells can be useful for the treatment of solid tumors. In some embodiments, inhibiting proliferation of one or more tumor cells can lead to one or more results including, but not limited to, transforming the one or more tumor cells into non-tumor cells, reducing the growth rate of a tumor containing the tumor cells, reducing the overall growth of the tumor containing the tumor cells, reducing the amount of tumor cells present in the tumor containing the tumor cells, reducing the accumulation of tumor cells in the tumor containing the tumor cells, reducing the capacity for the tumor cells to generate new tumor cells, or reducing the capacity for the tumor cells to divide or form new tumor cells. In some embodiments, transforming one or more tumor cells into non-tumor cells gives the tumor cell a finite life and strips the tumor cell of its capacity for tumor initiation, self-renewal, and differentiation. Accordingly, transforming one or more tumor cells into non-tumor cells may allow a patient receiving an COX-3 modulating agent of the present invention to outlive the non-tumor cells e.g., transformed tumor cells. In some embodiments, the method of inhibiting the proliferation of tumor cells eliminates e.g., kills, tumor cells in the tumor containing the tumor cells. In one embodiment, the method of inhibiting the proliferation of tumor cells is useful for increasing a patient's progression free survival time.

In some embodiments, inhibiting the proliferation of the one or more tumor cells includes inhibiting and/or eliminating a significant fraction of the tumor cells present in a tumor containing the tumor cells. In some embodiments, a significant fraction includes a majority of the tumor cell population present in the tumor. In some embodiments, a significant fraction comprises at least 1%, 10%, 20%, 30%, 40%, 50%, 60%, 70%, 80%, 85%, 90%, 95%, 96%, 97%, 98%, 99%, 99.5%, or up to about 100% of the tumor cells contained within a tumor. In one embodiment, a significant fraction includes up to about 100% of the tumor cell population present in the tumor. In one embodiment, a significant fraction includes 100% of the tumor cell population present in the tumor.

In some embodiments, the COX-3 modulating agent specifically inhibits the level and/or activity of COX-3 protein. In some embodiments, the COX-3 modulating agent specifically inhibits the level and/or activity of r68, r57, r50 and r44 proteins.

In some aspects, a method of inhibiting the growth and/or proliferation of one or more tumor cells which includes contacting the cell with both at least one COX-3 modulating agent. In some aspects, a method of inhibiting the proliferation of one or more tumor cell comprises contacting the cell with both at least two COX-3 modulating agents. In some embodiments, the contacting occurs simultaneously. In some embodiments, the contacting is occurs near simultaneously. In some embodiments, the COX-3 modulating agents specifically inhibit the level and/or activity of both COX-3 proteins.

In some aspects, the COX-3 modulating agent inhibits the level and/or activity of r68. In some embodiments, the COX-3 modulating agent specifically inhibits the level and/or activity of r57. In some embodiments, the COX-3 modulating agent specifically inhibits the level and/or activity of r50. In some embodiments, the COX-3 modulating agent specifically inhibits the level and/or activity of r44. Strategies for inhibiting the level and/or activity of COX-3 proteins can be performed by those of ordinary skill in the art without undue experimentation.

In some embodiments, the cell is a breast cell. In some embodiments, the cell is an ovarian cell. In some embodiments, the cell is a colon cell. In some embodiments, the cell is a brain cell. In some embodiments, the cell is a pancreatic cell. In some embodiments, the cell is a prostate cell. In some embodiments, the cell is a lung cell. In some embodiments, the cell is a solid tumor cell. In some embodiments, the cell is a hematological tumor cell. In some embodiments, the cell is obtained from a human subject.

### COX-3 Modulating Agents

The invention generally relates to a method of modulating autophagy in a cell comprising contacting one or more cells that expresses COX-3 with an effective amount of an agent that modulates the expression level and/or enzymatic activity of COX-3 or a component thereof. Examples of suitable COX-3 modulating agents that can be used for modulating autophagy include, but are not limited to nucleic acids e.g., antisense nucleic acids, oligopeptides, aptamers, ribozymes, small molecules, and antibodies or fragments thereof, and combinations thereof.

In some aspects, nucleic acids that can inhibit the expression and/or translation of COX-3 can also be used as modulating agents of COX-3. Such inhibitory nucleic acids can hybridize to a COX-3 nucleic acid under intracellular or stringent conditions. The inhibitory nucleic acid is capable of reducing expression or translation of a nucleic acid encoding COX-3. A nucleic acid encoding COX-3 may be genomic DNA as well as messenger RNA. It may be incorporated into a plasmid vector or viral DNA. It may be single strand or double strand, circular or linear.

An inhibitory nucleic acid is a polymer of ribose nucleotides or deoxyribose nucleotides having more than three nucleotides in length. An inhibitory nucleic acid may include naturally-occurring nucleotides; synthetic, modified, or pseudo-nucleotides such as phosphorothiolates; as well as nucleotides having a detectable label such as P³², biotin, fluorescent dye or digoxigenin. An inhibitory nucleic acid that can reduce the expression and/or activity of a COX-3 nucleic acid may be completely complementary to the COX-3 nucleic acid. Alternatively, some variability between the sequences may be permitted. In some embodiments, an inhibitory nucleic acid that can reduce the expression and/or activity of a COX-3 nucleic acid may be complementary to COX-3 nucleic acid variants. In some embodiments, the inhibitor nucleic acid may be complementary to r68, r57, r50, r44 nucleic acid or variants thereof to reduce the activity of COX-3 by reducing the expression and/or activity of r68, r57, r50, or r44.

An inhibitory nucleic acid of the invention can hybridize to a COX-3 nucleic acid under intracellular conditions or under stringent hybridization conditions. The inhibitory nucleic acids of the invention are sufficiently complementary to endogenous COX-3 nucleic acids to inhibit expression of a COX-3 nucleic acid under either or both conditions. Intracellular conditions refer to conditions such as temperature, pH and salt concentrations typically found inside a cell, e.g. a mammalian cell. One example of such a mammalian cell is a cancer cell (e.g., a tumor cell), or any cell where COX-3 is or may be expressed.

Generally, stringent hybridization conditions are selected to be about 5° C. lower than the thermal melting point (Tₘ) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory nucleic acids that comprise, for example, 2, 3, 4, or 5 or more stretches of contiguous nucleotides that are precisely complementary to a COX-3 coding sequence, each separated by a stretch of contiguous nucleotides that are not complementary to adjacent coding sequences, may inhibit the function of a COX-3 nucleic acid. In general, each stretch of contiguous nucleotides is at least 4, 5, 6, 7, or 8 or more nucleotides in length. Non-complementary intervening sequences may be 1, 2, 3, or 4 nucleotides in length. One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a ribozyme or an antisense nucleic acid molecule.

The antisense nucleic acid molecule may be single or double stranded (e.g. a small interfering RNA (siRNA)), and may function in an enzyme-dependent manner or by steric blocking. Antisense molecules that function in an enzyme-dependent manner include forms dependent on RNase H activity to degrade target mRNA. These include single-stranded DNA, RNA and phosphorothioate molecules, as well as the double-stranded RNAi/siRNA system that involves target mRNA recognition through sense-antisense strand pairing followed by degradation of the target mRNA by the RNA-induced silencing complex. Steric blocking antisense, which are RNase-H independent, interferes with gene expression or other mRNA-dependent cellular processes by binding to a target mRNA and interfering with other processes. Steric blocking antisense includes 2′-O alkyl (usually in chimeras with RNase-H dependent antisense), peptide nucleic acid (PNA), locked nucleic acid (LNA) and morpholino antisense.

Small interfering RNAs, for example, may be used to specifically reduce COX-3 translation such that the level of COX-3 polypeptide is reduced, siRNAs mediate post-transcriptional gene silencing in a sequence-specific manner. See, for example, http://www.ambion.com/techlib/hottopics/rnai/rnai_may2002_print.html. Once incorporated into an RNA-induced silencing complex, siRNA mediate cleavage of the homologous endogenous mRNA transcript by guiding the complex to the homologous mRNA transcript, which is then cleaved by the complex. The siRNA may be homologous to any region of the COX-3 mRNA transcript. The region of homology may be 30 nucleotides or less in length, less than 25 nucleotides, about 21 to 23 nucleotides in length or less, e.g., 19 nucleotides in length. SiRNA is typically double stranded and may have nucleotide 3′ overhangs. The 3′ overhangs may be up to about 5 or 6 nucleotide ‘3 overhangs, e.g., two nucleotide 3′ overhangs, such as, 3′ overhanging UU dinucleotides, for example. In some embodiments, the siRNAs may not include any nucleotide 3′ overhangs. Methods for designing siRNAs are known to those skilled in the art. See, for example, Elbashir et al. Nature 411: 494-498 (2001); Harborth et al. Antisense Nucleic Acid Drug Dev. 13: 83-106 (2003). Typically, a target site is selected that begins with AA, has 3′ UU overhangs for both the sense and antisense siRNA strands, and has an approximate 50% G/C content, siRNAs may be chemically synthesized, created by in vitro transcription, or expressed from an siRNA expression vector or a PCR expression cassette. See, e.g., http://www.ambion.com/techlib/tb/tb.sub.-506html.

When an siRNA is expressed from an expression vector or a PCR expression cassette, the insert encoding the siRNA may be expressed as an RNA transcript that folds into an siRNA hairpin. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be any appropriate length, for example, up to 30 nucleotides in length, e.g., 3 to 23 nucleotides in length, and may be of various nucleotide sequences. SiRNAs also may be produced in vivo by cleavage of double-stranded RNA introduced directly or via a transgene or virus. Amplification by an RNA-dependent RNA polymerase may occur in some organisms. The siRNA may be further modified according to any methods known to those having ordinary skill in the art.

An antisense inhibitory nucleic acid may also be used to specifically reduce COX-3 expression, for example, by inhibiting transcription and/or translation. An antisense inhibitory nucleic acid is complementary to a sense nucleic acid encoding COX-3. For example, it may be complementary to the coding strand of a double-stranded cDNA molecule or complementary to an mRNA sequence. It may be complementary to an entire coding strand or to only a portion thereof. It may also be complementary to all or part of the noncoding region of a nucleic acid encoding COX-3. The non-coding region includes the 5′ and 3′ regions that flank the coding region, for example, the 5′ and 3′ untranslated sequences. An antisense inhibitory nucleic acid is generally at least six nucleotides in length, but may be up to about 8, 12, 15, 20, 25, 30, 35, 40, 45, or 50 nucleotides long, Longer inhibitory nucleic acids may also be used.

An antisense inhibitory nucleic acid may be prepared using methods known in the art, for example, by expression from an expression vector encoding the antisense inhibitory nucleic acid or from an expression cassette. Alternatively, it may be prepared by chemical synthesis using naturally-occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the antisense inhibitory nucleic acid and the sense nucleic acid.

Naturally-occurring nucleotides include the ribose or deoxyribose nucleotides adenosine, guanine, cytosine, thymine and uracil. Examples of modified nucleotides include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladeninje, uracil-5oxyacetic acid, butoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl)uracil, (acp3)w, and 2,6-diaminopurine.

Thus, inhibitory nucleic acids of the invention may include modified nucleotides, as well as natural nucleotides such as combinations of ribose and deoxyribose nucleotides, and an antisense inhibitory nucleic acid of the invention may be of any length discussed above and that is complementary to the nucleic acid sequences of COX-3 or variants thereof. In some embodiments, the antisense inhibitory nucleic acids of the invention may be of any length discussed above and that is complementary to the nucleic acid sequences of r68, r57, r50, r44 or variants thereof.

In some embodiments, a COX-3 modulating agent of the present invention is a small hairpin RNA or short hairpin RNA (shRNA), shRNA is a sequence of RNA that makes a tight hairpin turn that can be used to silence gene expression by means of RNA interference. The shRNA hairpin structure is cleaved by the cellular machinery into a siRNA, which then binds to and cleaves the target mRNA. shRNA can be introduced into cells via a vector encoding the shRNA, where the shRNA coding region is operably linked to a promoter. The selected promoter permits expression of the shRNA. For example, the promoter can be a U6 promoter, which is useful for continuous expression of the shRNA. The vector can, for example, be passed on to daughter cells, allowing the gene silencing to be inherited. See, McIntyre G, Fanning G, Design and cloning strategies for constructing shRNA expression vectors, BMC BiOTECHNOL. 6:i (2006); Paddison et al., Short hairpin RNAs (shRNAs) induce sequence-specific silencing in mammalian cells, GENES DEV. 16 (8): 948-58 (2002).

In some embodiments, a COX-3 modulating agent of the present invention is a ribozyme. A ribozyme is an RNA molecule with catalytic activity and is capable of cleaving a single-stranded nucleic acid such as an mRNA that has a homologous region. See, for example, Cech, Science 236: 1532-1539 (1987); Cech, Ann. Rev. Biochem. 59:543-568 (1990); Cech, Curr. Opin. Struct. Biol. 2: 605-609 (1992); Couture and Stinchcomb, Trends Genet. 12: 510-515 (1996). A ribozyme may be used to catalytically cleave a COX-3 mRNA transcript and thereby inhibit translation of the mRNA. See, for example, Haseloff et al., U.S. Pat. No. 5,641,673. A ribozyme having specificity for a COX-3 nucleic acid or variant thereof may be designed based on the publicly available nucleotide sequences of COX-3.

Methods of designing and constructing a ribozyme that can cleave an RNA molecule in trans in a highly sequence specific manner have been developed and described in the art. See, for example, Haseloff et al., Nature 334:585-591 (1988). A ribozyme may be targeted to a specific RNA by engineering a discrete “hybridization” region into the ribozyme. The hybridization region contains a sequence complementary to the target RNA that enables the ribozyme to specifically hybridize with the target. See, for example, Gerlach et al., EP 321,201. The target sequence may be a segment of about 5, 6, 7, 8, 9, 10, 12, 15, 20, or 50 contiguous nucleotides selected from the nucleotide sequence of COX-3 and/or r68 and/or r57 and/or r50-1 and/or r44. Longer complementary sequences may be used to increase the affinity of the hybridization sequence for the target.

The hybridizing and cleavage regions of the ribozyme can be integrally related; thus, upon hybridizing to the target RNA through the complementary regions, the catalytic region of the ribozyme can cleave the target. Thus, an existing ribozyme may be modified to target a COX-3 nucleic acid of the invention by modifying the hybridization region of the ribozyme to include a sequence that is complementary to the target COX-3 nucleic acid. Alternatively, an mRNA encoding COX-3 may be used to select a catalytic RNA having a specific ribonuclease activity from a pool of RNA molecules. See, for example, Bartel & Szostak, Science 261:1411-1418 (1993).

In some aspects, the COX-3 modulating agents of the present invention comprise a protein or polypeptide COX-3 binding molecule, in some embodiments, the COX-3 modulating agents of the present invention comprise a protein or polypeptide r68 and/or r57-1 and/or r50 and/or 44 binding molecule. In some embodiments, the binding molecules bind directly and specifically to a target polypeptide e.g., COX-3 and/or r68 and/or r57 and/or r50 and/or r40, and interfere with the enzymatic activity of the target polypeptide.

Exemplary protein or polypeptide COX-3 binding molecules preferably have little or no binding affinity for non-COX-3 proteins.

In some embodiments, the COX-3 modulating agents of the present invention may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. COX-3 and/or r68 and/or r57 and/or r50 and/or r44 binding molecules may have both a heavy and a light chain. Preferred COX-3 and/or r68 and/or r57 and/or r50 and/or r44 modulating agents of the present invention include, antibodies (including full length antibodies), monoclonal antibodies (including full length monoclonal antibodies), polyclonal antibodies, multispecific antibodies (e.g., bispecific antibodies), human, humanized or chimeric antibodies, and antibody fragments, e.g., Fab fragments, F(ab′) fragments, fragments produced by a Fab expression library, epitope-binding fragments of any of the above, and engineered forms of antibodies, e.g., scFv molecules, so long as they exhibit the desired activity, e.g., binding to COX-3 and/or r68 and/or r57 and/or r50 and/or r44.

Anti-COX-3 and/or anti-r68 and/or anti-r57 and/or anti-r50 and/or anti-r44 antibodies can be produced using any of the commonly utilized methods for generating antibodies known to those in the art. Procedures for raising polyclonal antibodies are well known in the art. Typically, such antibodies are raised by immunizing an animal (e.g. a rabbit, rat, mouse, donkey, etc) by multiple subcutaneous or intraperitoneal injections of the relevant antigen (a purified COX-3 and/or r68 and/or r57 and/or r50 and/or r44 peptide fragment, full-length recombinant COX-3 and/or r68 and/or r57 and/or r50 and/or r44 protein, fusion protein, etc) optionally conjugated to keyhole limpet hemocyanin (KLH), serum albumin, other immunogenic carrier, diluted in sterile saline and combined with an adjuvant (e.g. Complete or Incomplete Freund's Adjuvant) to form a stable emulsion. The polyclonal antibody is then recovered from blood or ascites of the immunized. Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, etc. Polyclonal antiserum can also be rendered monospecific using standard procedures (See e.g. Agaton et al., “Selective Enrichment of Monospecific Polyclonal Antibodies for Antibody-Based Proteomics Efforts,” J Chromatography A 1043(1):33-40 (2004), which is hereby incorporated by reference in its entirety).

In some embodiments, monoclonal antibodies can be prepared using hybridoma methods, such as those described by Kohler and Milstein, “Continuous Cultures of Fused Cells Secreting Antibody of Predefined Specificity,” Nature 256:495-7 (1975), which is hereby incorporated by reference in its entirety, Using the hybridoma method, a mouse, hamster, or other appropriate host animal, is immunized to elicit the production by lymphocytes of antibodies that will specifically bind to an immunizing antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against COX-3, as determined by immunoprecipitation, immunoblotting, or by an in vitro binding assay such as radioimmunoassay (RIA) or enzyme-linked immunosorbent assay (ELISA) can then be propagated either in vitro culture using standard methods (James Goding, MONOCLONAL ANTIBODIES: PRINCIPLES AND PRACTICE (1986) which is hereby incorporated by reference in its entirety) or in vivo as ascites tumors in an animal. The monoclonal antibodies can then be purified from the culture medium or ascites fluid as described for polyclonal antibodies above.

In some embodiments, monoclonal antibodies can be made using recombinant DNA methods as described in U.S. Pat. No. 4,816,567 to Cabilly et al, which is hereby incorporated by reference in its entirety. The polynucleotides encoding a monoclonal antibody are isolated, such as from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, and monoclonal antibodies are generated by the host cells. Recombinant monoclonal antibodies or fragments thereof of the desired species can also be isolated from phage display libraries as described (McCafferty et al., “Phage Antibodies: Filamentous Phage Displaying Antibody Variable Domains,” Nature 348:552-554 (1990); Clackson et al., “Making Antibody Fragments using Phage Display Libraries,” Nature 352:624-628 (1991); and Marks et al., “By-Passing Immunization. Human Antibodies from V-Gene Libraries Displayed on Phage,” J. Mol. Biol. 222:581-597 (1991), which are hereby incorporated by reference in their entirety).

The polynucleotide(s) encoding a monoclonal antibody can further be modified in a number of different ways using recombinant DNA technology to generate alternative antibodies. In one embodiment, the constant domains of the light and heavy chains of, for example, a mouse monoclonal antibody can be substituted for those regions of a human antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

In some embodiments, the monoclonal antibody against COX-3 and/or r68 and/or r57 and/or r50 and/or r44 is a humanized antibody. Humanized antibodies are antibodies that contain minimal sequences from non-human (e.g. murine) antibodies within the variable regions. Such antibodies are used therapeutically to reduce antigenicity and human anti-mouse antibody responses when administered to a human subject. In practice, humanized antibodies are typically human antibodies with minimum to no non-human sequences. A human antibody is an antibody produced by a human or an antibody having an amino acid sequence corresponding to an antibody produced by a human.

Humanized antibodies can be produced using various techniques known in the art. An antibody can be humanized by substituting the complementarity determining region (CDK) of a human antibody with that of a non-human antibody (e.g. mouse, rat, rabbit, hamster, etc.) having the desired specificity, affinity, and capability (Jones et al., “Replacing the Complementarity-Determining Regions in a Human Antibody With Those From a Mouse,” Nature 321:522-525 (1986); Riechmann et al., “Reshaping Human Antibodies for Therapy,” Nature 332:323-327 (1988); Verhoeyen et al., “Reshaping Human Antibodies: Grafting an Antilysozyme Activity,” Science 239:1534-1536 (1988), which are hereby incorporated by reference in their entirety). The humanized antibody can be further modified by the substitution of additional residues either in the Fv framework region and/or within the replaced non-human residues to refine and optimize antibody specificity, affinity, and/or capability.

Human antibodies can be directly prepared using various techniques known in the art. Immortalized human B lymphocytes immunized in vitro or isolated from an immunized individual that produce an antibody directed against a target antigen can be generated (See e.g. Reisfeld et al., Monoclonal Antibodies and Cancer Therapy 77 (Alan R. Liss 1985) and U.S. Pat. No. 5,750,373 to Garrard, which are hereby incorporated by reference in their entirety). Also, the human antibody can be selected from a phage library, where that phage library expresses human antibodies (Vaughan et al., “Human Antibodies with Sub-Nanomolar Affinities Isolated from a Large Non-immunized Phage Display Library,” Nature Biotechnology, 14:309-314 (1996); Sheets et al., “Efficient Construction of a Large Nonimmune Phage Antibody Library: The Production of High-Affinity Human Single-Chain Antibodies to Protein Antigens,” Proc Nat'l Acad Sci USA 95:6157-6162 (1998); Hoogenboom et al., “By-passing Immunisation. Human Antibodies From Synthetic Repertoires of Germline VH Gene Segments Rearranged In Vitro,” J Mol. Biol, 227:381-8 (1992); Marks et al., “By-passing Immunization. Human Antibodies from V-gene Libraries Displayed on Phage,” J. Mol. Biol, 222:581-97 (1991), which are hereby incorporated by reference in their entirety). Humanized antibodies can also be made in transgenic mice containing human immunoglobulin loci that are capable upon immunization of producing the full repertoire of human antibodies in the absence of endogenous immunoglobulin production. This approach is described in U.S. Pat. No. 5,545,807 to Surani et al.; U.S. Pat. No. 5,545,806 to Lonberg et al.; U.S. Pat. No. 5,569,825 to Lonberg et al.; U.S. Pat. No. 5,625,126 to Lonberg et al.; U.S. Pat. No. 5,633,425 to Lonberg et al.; and U.S. Pat. No. 5,661,016 to Lonberg et al., which are hereby incorporated by reference in their entirety.

In some embodiments, the COX-3 modulating agents of the present invention include bispecific antibodies that specifically recognize COX-3 and/or r68 and/or r57 and/or r50 and/or r44. Bispecific antibodies are antibodies that are capable of specifically recognizing and binding at least two different epitopes. Bispecific antibodies can be intact antibodies or antibody fragments. Techniques for making bispecific antibodies are common in the art (Brennan et al., “Preparation of Bispecific Antibodies by Chemical Recombination of Monoclonal Immunoglobulin G1 Fragments,” Science 229:81-3 (1985); Suresh et al, “Bispecific Monoclonal Antibodies From Hybrid Hybridomas,” Methods in Enzymol. 121:210-28 (1986); Traunecker et al., “Bispecific Single Chain Molecules (Janusins) Target Cytotoxic Lymphocytes on HIV Infected Cells,” EMBO J. 10:3655-3659 (1991); Shalaby et al., “Development of Humanized Bispecific Antibodies Reactive with Cytotoxic Lymphocytes and Tumor Cells Overexpressing the HER2 Protooncogene,” J. Exp. Med. 175:217-225 (1992); Kostelny et al, “Formation of a Bispecific Antibody by the Use of Leucine Zippers,” J. Immunol. 148: 1547-1553 (1992); Gruber et al., “Efficient Tumor Cell Lysis Mediated by a Bispecific Single Chain Antibody Expressed in Escherichia coli,” J. Immunol. 152:5368-74 (1994); and U.S. Pat. No. 5,731,168 to Carter et al., which are hereby incorporated by reference in their entirety).

In certain embodiments, it may be desirable to use an antibody fragment, rather than an intact antibody, for example, to increase tumor penetration. Various techniques are known for the production of antibody fragments. Traditionally, these fragments are derived via proteolytic digestion of intact antibodies (e.g. Morimoto et al., “Single-step Purification of F(ab′)2 Fragments of Mouse Monoclonal Antibodies (immunoglobulins G1) by Hydrophobic Interaction High Performance Liquid Chromatography Using TSKgel Phenyl-5PW,” Journal of Biochemical and Biophysical Methods 24:107-117 (1992) and Brennan et al., “Preparation of Bispecific Antibodies by Chemical Recombination of Monoclonal Immunoglobulin G1 Fragments,” Science 229:81-3 (1985), which are hereby incorporated by reference in their entirety). However, these fragments are now typically produced directly by recombinant host cells as described above. Thus Fab, Fv, and scFv antibody fragments can all be expressed in and secreted from E. coli or other host cells, thus allowing the production of large amounts of these fragments. Alternatively, such antibody fragments can be isolated from the antibody phage libraries discussed above. The antibody fragment can also be linear antibodies as described in U.S. Pat. No. 5,641,870 to Rinderknecht et al., which is hereby incorporated by reference, and can be monospecific or bispecific. Other techniques for the production of antibody fragments will be apparent to the skilled practitioner.

It may further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

The present invention further encompasses variants and equivalents which are substantially homologous to the chimeric, humanized and human antibodies, or antibody fragments thereof. These can contain, for example, conservative substitution mutations, i.e. the substitution of one or more amino acids by similar amino acids, which maintain or improve the binding activity of the antibody or antibody fragment.

In some embodiments, CONX-3 inhibitors of the present invention include antibody mimics. A number of antibody mimics are known in the art including, without limitation, those known as monobodies, which are derived from the tenth human fibronectin type III domain (.sup.10Fn3) (Koide et al., “The Fibronectin Type III Domain as a Scaffold for Novel Binding Proteins,” J. Mol. Biol. 284:1141-1151 (1998); Koide et al., “Probing Protein Conformational Changes in Living Cells by Using Designer Binding Proteins: Application to the Estrogen Receptor,” Proc. Nat'l Acad. Sci. USA 99:1253-1258 (2002), which are hereby incorporated by reference in their entirety); and those known as affibodies, which are derived from the stable .alpha.-helical bacterial receptor domain Z of staphylococcal protein A (Nord et al., “Binding Proteins Selected from Combinatorial Libraries of an a-Helical Bacterial Receptor Domain,” Nat. Biotechnol. 15(8):772-777 (1997), which is hereby incorporated by reference in its entirety). Variations in these antibody mimics can be created by substituting one or more domains of these polypeptides with a COX-3 and/or r68 and/or r57 and/or r50 and/or r44 specific domain and then screening the modified monobodies or affibodies for specificity for binding to COX-3 and/or r68 and/or r57 and/or r50 and/or r44.

In some embodiments, COX-3 modulating agents of the present invention include COX-3 and/or r68 and/or r57 and/or r50 and/or r44 binding oligopeptides. A COX-3 and/or r68 and/or r57 and/or r50 and/or r44-binding oligopeptide is an oligopeptide that binds, preferably specifically to the COX-3 and/or r68 and/or r57 and/or r50 and/or r44 protein. Such oligopeptides may be chemically synthesized using known oligopeptide synthesis methodology or may be prepared and purified using recombinant technology. Such oligopeptides are usually at least about 5 amino acids in length, but can be anywhere from 5 to 100 amino acids in length. Such oligopeptides may be identified without undue experimentation using well known techniques. Techniques for screening oligopeptide libraries for oligopeptides that are capable of specifically binding to a polypeptide target are well known in the art.

In some embodiments, a COX-3 modulating agent of the present invention is administered in combination with a cancer therapeutic agent. In some embodiments, the COX-3 modulating agent of the present invention is administered to a patient undergoing conventional chemotherapy and/or radiotherapy e.g., to inhibit the proliferation of, or to eliminate the tumor cells remaining after receiving the conventional cancer therapy. In some embodiments, the cancer therapeutic agent is a chemotherapeutic agent. In some embodiments, the cancer therapeutic agent is an immunotherapeutic agent. In some embodiments, the cancer therapeutic agent is a radiotherapeutic agent.

In certain embodiments, an COX-3 modulating agent of the present invention is linked or conjugated to a cancer therapeutic agent to facilitate direct delivery of the therapeutic agent to the solid tumor e.g., breast, ovarian, colon, etc. In an embodiment, the COX-3 modulating agent is an antibody that is linked or conjugated to a cancer therapeutic. Methods of making such conjugates, in particular antibody-drug conjugates, are known in the art and are described in WO2005/077090 to Duffy et al., WO2005/082023 to Feng, WO2005/084390 to Alley et al., WO2006/065533 to McDonagh et al., WO2007/103288 to McDonagh et al., WO2007/011968 to Jeffery and WO2008/070593 to McDonagh et al., which are all hereby incorporated by reference in their entirety. Cancer therapeutics that can be linked to the COX-3 modulating agent include, but are not limited to, chemotherapeutic agents or immunotherapeutic agents.

Exemplary chemotherapeutic agents include the toxins, diphtheria, ricin, and cholera toxin. Other chemotherapeutic agents that can be linked to the COX-3 modulating agents of the present invention include alkylating agents (e.g. cisplatin, carboplatin, oxaloplatin, mechlorethamine, cyclophosphamide, chorambucil, nitrosureas); anti-metabolites (e.g. methotrexate, pemetrexed, 6-mercaptopurine, dacarbazine, fludarabine, 5-fluorouracil, arabinosycytosine, capecitabine, gemcitabine, decitabine); plant alkaloids and terpenoids including vinca alkaloids (e.g. vincristine, vinblastine, vinorelbine), podophyllotoxin (e.g. etoposide, teniposide), taxanes (e.g. paclitaxel, docetaxel); topoisomerase inhibitors (e.g. notecan, topotecan, amasacrine, etoposide phosphate); antitumor antibiotics (dactinomycin, doxorubicin, epirubicin, and bleomycin); ribonucleotides reductase inhibitors; antimicrotubules agents; and retinoids.

In some embodiments, the COX-3 modulating agents of the present invention are linked to an immunotherapeutic agent. The immunotherapeutic agent can be a cytokine. The cytokine is exemplified by interleukin-1 (IL-I), IL-2, IL-4, IL-5, IL-Iβ, IL-7, IL-10, IL-12, IL-15, IL-18, CSF-GM, CSF-G, IFN-γ, IFN-α, TNF, TGF-β but not always limited thereto.

In some embodiments, the COX-3 modulating agents of the present invention can be linked or conjugated to a delivery vehicle containing a cancer therapeutic. Suitable delivery vehicles include liposomes (Hughes et al., “Monoclonal Antibody Targeting of Liposomes to Mouse Lung In Vivo,” Cancer Res 49(22):6214-20 (1989), which is hereby incorporated by reference in its entirety), nanoparticles (Farokhzad et al., “Targeted Nanoparticle-Aptamer Bioconjugates for Cancer Chemotherapy In Viva,” Proc Nat'l Acad Sci USA 103(16):6315-20(2006), which is hereby incorporated by reference in its entirety), biodegradable microspheres, microparticles, and collagen minipellets. The delivery vehicle can contain any of the chemotherapeutic, radiotherapeutic, or immunotherapeutic agents described supra.

In one embodiment, the COX-3 modulating agents of the present invention are conjugated to a liposome delivery vehicle (Sofou & Sgouros, “Antibody-Targeted Liposomes in Cancer Therapy and Imaging,” Exp Opin Drug Deliv 5(2):189-204 (2008), which is hereby incorporated by reference in its entirety). Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug (cancer therapeutic) at the primary solid tumor site. This can be accomplished, for example, in a passive manner where the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Wang & Huang, “pH-Sensitive Immunoliposomes Mediate Target-cell-specific Delivery and Controlled Expression of a Foreign Gene in Mouse,” Proc. Nat'l Acad. Sci. USA 84:7851-5 (1987), which is hereby incorporated by reference in its entirety). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane, which enzyme slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release.

Different types of liposomes can be prepared according to Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda; and U.S. Pat. No. 5,059,421 to Loughrey et al., which are hereby incorporated by reference in their entirety.

These liposomes can be produced such that they contain, in addition to the therapeutic agents of the present invention, other therapeutic agents, such as immunotherapeutic cytokines, which would then be released at the target site (e.g., Wolff et al., “The Use of Monoclonal Anti-Thy1 IgG1 for the Targeting of Liposomes to AKR-A Cells in Vitro and in Vivo,” Biochim. Biophys. Acta 802:259-73 (1984), which is hereby incorporated by reference in its entirety).

### III. TREATMENT METHODS USING AGENTS TO TARGET COX-3

Methods of the invention are directed to the use of modulating agents that target COX-3, e.g., antibodies, including antisense nucleic acids, oligopeptides, aptamers, ribozymes, small molecules, and antibodies or fragments thereof, and combinations thereof, to modulate diseases, disorders, or conditions mediated by or involving COX-3.

In one embodiment, treatment includes the application or administration of a COX-3 modulating agent as described herein to a patient to treat or prevent autophagy. In another embodiment, treatment is also intended to include the application or administration of a pharmaceutical composition comprising the COX-3 modulating agent to a patient to treat or prevent autophagy. It should be appreciated that due to the interaction of COX-3 with Nuc, the application or administration of a COX-3 modulating agent is expected to interfere with the interaction of COX-3 and Nuc.

The COX-3 modulating agents as described herein are useful for the treatment or prevention of autophagy. In some embodiments, treatment or preventing of autophagy is intended to include an inhibition of tumor cell proliferation, a reduction in the number of tumor cells, elimination of tumor cells, a reduction in tumor growth, and a reduction in tumor size or bulk. In one embodiment, administration of the COX-3 modulating agent diminishes tumor invasion and migration (i.e. tumor metastasis) thereby delaying or inhibiting tumor progression. Administration of the COX-3 modulating agent alone, in combination with a cancer therapeutic agent or linked to a cancer therapeutic agent alleviates one or more of the symptoms associated with the solid tumor and reduces or prevents morbidity and mortality of the subject having the solid tumor.

In one embodiment, the invention relates to the use of COX-3 modulating agents as a medicament, in particular for use in the treatment or prevention of autophagy. In accordance with the methods of the present invention, at least COX-3 modulating agent as defined herein can be used to promote a positive therapeutic response with respect to autophagy. A “positive therapeutic response” with respect to autophagy is intended to include an improvement in the disease in association with autophagy, and/or an improvement in the symptoms associated with the disease. That is, an anti-proliferative effect, the prevention of further proliferation of the COX-3-expressing cell, a reduction in the inflammatory response including but not limited to reduced secretion of inflammatory cytokines, adhesion molecules, proteases, immunoglobulins (in instances where the SEMA4D bearing cell is a B cell), combinations thereof, and the like, increased production of anti-inflammatory proteins, a reduction in the number of autoreactive cells, an increase in immune tolerance, inhibition of autoreactive cell survival, reduction in apoptosis, reduction in endothelial cell migration, increase in spontaneous monocyte migration, reduction in and/or a decrease in one or more symptoms mediated by stimulation of COX-3-expressing cells can be observed. Such positive therapeutic responses are not limited to the route of administration and may comprise administration to the donor, the donor tissue (such as for example organ perfusion), the host, any combination thereof, and the like. In particular, the methods provided herein are directed to inhibiting, preventing, reducing, alleviating, or lessening the development of a tumor size in a patient. Thus, for example, an improvement in the disease may be characterized as an absence of clinically observable symptoms, a decrease in tumor cell proliferation, a reduction in the number of tumor cells, elimination of tumor cells, a reduction in tumor growth, and a reduction in tumor size or bulk.

### IV. PHARMACEUTICAL COMPOSITIONS AND ADMINISTRATION METHODS

In some aspects, the present invention provides pharmaceutical compositions comprising the COX-3 modulating agent alone, the COX-3 modulating agent in combination with a cancer therapeutic agent, or the COX-3 modulating agent conjugated to a cancer therapeutic agent, and/or the COX-3 modulating agent component linked to a delivery vehicle, which are suitable for treating a solid tumor. In some embodiments, the present invention provides pharmaceutical compositions comprising the COX-3 modulating agent alone, the COX-3 modulating agent in combination with a cancer therapeutic agent, or the COX-3 modulating agent conjugated to a cancer therapeutic agent, and/or the COX-3 modulating agent component linked to a delivery vehicle, which are suitable for treating a hematological tumor. Therapeutic formulations of the COX-3 modulating agents (e.g. COX-3 antibodies or antibody fragments, binding oligopeptides, COX-3 RNAi or antisense molecules, and COX-3 binding small molecules) are prepared for storage by mixing the antibody, oligopeptide, nucleic acid or small molecule having the desired degree of purity with optional pharmaceutically acceptable carriers, excipients or stabilizers (REMINGTON'S PHARMACEUTICAL SCIENCES (A. Osol ed. 1980), which is hereby incorporated by reference in its entirety), in the form of lyophilized formulations or aqueous solutions. Acceptable carriers, excipients, or stabilizers are nontoxic to recipients at the dosages and concentrations employed, and include buffers such as acetate, Tris-phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWEEN®, PLURONICS® or polyethylene glycol (PEG).

The active therapeutic ingredients of the pharmaceutical compositions (i.e. COX-3 modulating agents alone or linked to a cancer therapeutic agent) can be entrapped in microcapsules prepared using coacervation techniques or by interfacial polymerization, e.g., hydroxymethylcellulose or gelatin-microcapsules and poly-(methylmethacylate) microcapsules, respectively, in colloidal drug delivery systems (e.g., liposomes, albumin microspheres, microemulsions, nano-particles and nanocapsules) or in macroemulsions. Such techniques are disclosed in REMINGTON'S PHARMACEUTICAL SCIENCES (A. Osol ed. 1980), which is hereby incorporated by reference in its entirety. In some embodiments, the COX-3 modulating agents of the present invention can be conjugated to the microcapsule delivery vehicle to target the delivery of the therapeutic agent to the site of the tumor. Sustained-release preparations may be prepared. Suitable examples of sustained-release preparations include semi-permeable matrices of solid hydrophobic polymers containing the antibody or polypeptide, which matrices are in the form of shaped articles, e.g., films or microcapsules. Examples of sustained-release matrices include polyesters, hydrogels (for example, poly(2-hydroxyethyl-methacrylate), or poly(vinylalcohol)), polylactides, copolymers of L-glutamic acid and .gamma. ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid.

The therapeutically effective compositions containing the COX-3 modulating agents of the present invention are administered to a subject, in accordance with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes.

Other therapeutic regimens may be combined with the administration of the COX-3 modulating agents. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

In some embodiments, it may also be desirable to combine administration of the COX-3 modulating agent with administration of an antibody directed against another tumor antigen associated with the solid tumor.

In another embodiment, the therapeutic treatment methods of the present invention involve the combined administration of one or more COX-3 modulating agents, in combination with a cancer therapeutic agent, or conjugated to a distinct chemotherapeutic agent, radiotherapeutic agent, or immunotherapeutic agent, resulting in the administration of a cocktail of chemotherapeutic, radiotherapeutic, and/or immunotherapeutic agents. In another embodiment, the COX-3 modulating agents alone or conjugated to the cancer therapeutic can be administered with one or more additional chemotherapeutic agents. Preparation and dosing schedules for such chemotherapeutic agents may be used according to manufacturers' instructions or as determined empirically by the skilled practitioner. Preparation and dosing schedules for such chemotherapy are also described in CHEMOTHERAPY SERVICE (M. C. Perry ed., 1992), which is hereby incorporated by reference in its entirety.

For the treatment of a solid tumor or a hematological tumor, the dosage and mode of administration will be chosen by the physician according to known criteria. A therapeutically effective dose of the COX-3 modulating agent alone or linked to a cancer therapeutic agent is the amount effective for inhibiting the proliferation of tumor cells, reducing tumor cells e.g., killing tumor cells or non-tumor cells, reducing tumor size, reducing tumor cell migration and invasion, or reducing tumor growth. The dosage should not cause adverse side effects, such as unwanted cross-reactions, anaphylactic reactions, and the like. Generally, the dosage will vary with the age, condition, sex and route of administration, or whether other drugs are included in the regimen, and can be determined by one of skill in the art. The appropriate dosage of the COX-3 modulating agent will also depend on the type of solid tumor (e.g., breast, ovarian, colon, brain, pancreatic, etc.) or hematological tumor (e.g., leukemia, myeloma, lymphoma, etc.) to be treated and the severity and course of the disease. The COX-3 modulating agent may be appropriately administered to the patient at one time or over a series of treatments.

In some aspects, the present invention provides a method for treating a tumor that includes administering to an individual in need thereof an effective amount of an agent which specifically inhibits the activity or level of COX-3 protein. In some embodiments, treating the tumor inhibits the proliferation of tumor cells and/or eliminates the tumor cells.

In some embodiments, the agent specifically inhibits the level and/or activity of r68. In some embodiments, the agent specifically inhibits the level and/or activity of r57. In some embodiments, the agent specifically inhibits the level and/or activity of r50. In some embodiments, the agent specifically inhibits the level and/or activity of r44.

As discussed supra, examples of suitable agents that can be used for treating tumors include, but are not limited to nucleic acids e.g., antisense nucleic acids, oligopeptides, aptamers, ribozymes, small molecules, and antibodies or fragments thereof, and combinations thereof.

In some embodiments, the agent is administered with a pharmaceutically acceptable carrier. In some embodiments, the agent is co-administered with at least one additional chemotherapeutic agent, as described above.

In some embodiments, the agent includes interfering RNA targeted to COX-3 in the individual, which interferes with COX-3 expression within the individual, as described elsewhere herein. In certain embodiments, the interfering RNA is an siRNA. In certain embodiments, the interfering RNA is a small hairpin RNA.

In some embodiments, the agent includes an oligonucleotide having a nucleotide sequence that is complementary to COX-3 mRNA within the individual. In certain embodiments, the oligonucleotide is within the range of about 5 to about 50 nucleotides in length.

In some embodiments, the tumor is a solid tumor. In some embodiments, the solid tumor is one of a breast tumor, ovarian tumor, colon tumor, brain tumor, pancreatic tumor, prostate tumor, or lung tumor. In some embodiments, the tumor is a hematological tumor e.g., leukemia, myeloma, lymphoma, etc.

In some aspects, the invention provides a method for treating cancer that includes administering to an individual in need thereof an effective amount of an agent which specifically inhibits the activity or level of COX-3. In some embodiments, the method of treating cancer is useful for inhibiting the proliferation of tumor cells. In some embodiments, the method of treating cancer is useful for eliminating tumor cells e.g., killing tumor cells.

In some embodiments, the cancer is characterized as one in which one or more cancerous cells produce an increased level and/or activity of COX-3 protein. In certain embodiments, the cancerous cells are tumor cells. In certain embodiments, the tumor cells are breast tumor cells. In certain embodiments, the tumor cells are ovarian tumor cells. In certain embodiments, the tumor cells are colon tumor cells.

In some embodiments, the agent specifically inhibits the level or activity of r68. In some embodiments, the agent specifically inhibits the level or activity of r57. In some embodiments, the agent specifically inhibits the level and/or activity of r50. In some embodiments, the agent specifically inhibits the level and/or activity of r44.

In some embodiments, the agent specifically inhibits the activity or level of COX-3 in a tumor cell.

In some embodiments, the agent is an agent which downregulates COX-3 gene expression, inhibits COX-3 translation, inhibits COX-3 protein activity, and/or reduces the level of COX-3 protein.

In some embodiments, the agent is an agent that inhibits transcription of COX-3 mRNA, degrades COX-3 mRNA, inhibits translation of COX-3 mRNA, and combinations thereof. In an embodiment, the agent that inhibits transcription of COX-3 mRNA is an interfering RNA (RNAi). In an embodiment, the agent that degrades COX-3 mRNA comprises an interfering RNA (RNAi). In an embodiment, the agent that inhibits translation of COX-3 mRNA includes an antisense nucleic acids, a ribozyme, and combinations thereof as discussed in detail above. In an embodiment, the agent that modulates COX-3 activity is an siRNA targeted to COX-3. In an embodiment, the agent that downregulates COX-3 expression comprises a small molecule inhibitor of COX-3. In an embodiment, the agent that downregulates COX-3 expression is an siRNA or pharmacologic agent capable of inhibiting COX-3 gene expression.

### V. COMPOUNDS AND METHODS FOR IDENTIFYING COMPOUNDS

The invention provides methods of identifying compounds or agents for modulating autophagy. Further provided are compositions useful for performing the inventive methods. In some aspects, the invention provides a method of identifying a compound that modulates COX-3 expression or activity, the method comprising a) expressing COX-3 protein in a cell or population of cells; b) contacting said cell or population with said candidate agent; and c) measuring the level of expression or activity of COX-3; wherein a decrease in expression or activity of the COX-3 protein relative to a control cell population not exposed to said candidate agent is indicative of COX-3 modulating activity of said candidate agent. In some aspects, the COX-3 protein is selected from the group consisting of r68, r57, r50 or r44. The candidate agent selected may be a small molecule or may be selected from the group consisting of an antisense oligonucleotide, an oligopeptide, a ribozyme, an siRNA, a ribozyme, an aptamer, and an antibody or a fragment thereof. Such compounds may be useful, e.g., to inhibit viral replication, e.g., for treatment of disorders involving excessive replication, such as encephalomyocarditis.

The invention further provides methods of identifying a candidate agent that modulates autophagy in a cell comprising: a) contacting a cell or population of cells that expresses COX-3 protein with a candidate autophagy modulating agent; and b) measuring the level of expression and/or enzymatic activity of COX-3, wherein: i) a decrease in expression and/or enzymatic activity of COX-3 protein relative to a control cell or population of cells not exposed to said candidate autophagy modulating agent is indicative that said candidate autophagy modulating agent inhibits autophagy; or ii) an increase in expression and/or enzymatic activity of COX-3 protein relative to a control cell or population of cells not exposed to said candidate autophagy modulating agent is indicative that said candidate autophagy modulating agent induces autophagy. Potential markers for COX-3 include, but are not limited to, Myc, FLAG, and the amphisome marker LAMP-1. Compounds identified using an inventive method may further be used to assess autophagy of the cell or cell population.

The invention further provides methods of identifying compounds useful in inhibiting encephalomyocarditis viral (EMCV) replication comprising: a) providing a composition comprising a COX-3 polypeptide and a candidate agent; (b) determining whether the candidate agent inhibits the COX-3 polypeptide; wherein if the candidate agent inhibits the COX-3 polypeptide, the candidate agent is identified as a candidate agent that inhibits EMCV replication. The method may further include assessing the ability of the candidate agent that inhibits EMCV replication to inhibit EMCV viral replication. In some embodiments, the determining step may include determining whether the test compound inhibits (i) expression of the COX-3 polypeptide or (ii) enzymatic activity of the COX-3 polypeptide. In some embodiments, the composition of step a) may include i) a cell-free composition comprising purified COX-3, and wherein step (b) comprises determining whether the candidate agent inhibits enzymatic activity of COX-3; or ii) a cell that expresses a COX-3 polypeptide, and wherein step (b) comprises determining whether the candidate agent inhibits expression or enzymatic activity of COX-3. The method may further include i) contacting a cell with the candidate agent and a virus, wherein the cell would be susceptible to the virus in the absence of the candidate agent; and/or ii) administering the candidate agent to a subject, wherein the subject would be susceptible to EMCV infection in the absence of the candidate agent; and/or iii) contacting a cell that is infected by EMCV with the candidate agent; and iv) administering the candidate agent to a subject, wherein the subject is infected by EMCV.

Compounds identified using an inventive method may be used for any purpose in which it is desired to alter expression or activity of the gene product. In some embodiments, a compound is useful for increasing or decreasing production of a functional gene product of interest by cells. In some embodiments, the cells are isolated cells. In some embodiments, a compound is useful for increasing or decreasing production of a gene product in vivo.

A compound identified using an inventive method, e.g., a compound identified as a modulator of a DNA or gene product of interest, can be tested in cell culture or in animal models (“in vivo”) to further characterize its effects. Cytotoxicity can be assessed e.g., using any of a variety of assays for cell viability and/or proliferation such as a cell membrane integrity assay, a cellular ATP-based viability assay, a mitochondrial reductase activity assay, a BrdU, EdU, or H3-Thymidine incorporation assay, a DNA content assay using a nucleic acid dye, such as Hoechst Dye, DAPI, Actinomycin D, 7-aminoactinomycin D or propidium iodide, a cellular metabolism assay such as AlamarBlue, MTT, XTT, and CellTitre Glo, etc. The compound can be tested in an animal model of a disorder, e.g., a genetic disorder.

One of skill in the art would be aware of suitable methods to assess expression and/or activity of a gene product of interest. Methods known in the art can be used for measuring mRNA or protein. A variety of different hybridization-based or amplification-based methods are available to measure RNA. Examples include Northern blots, microarray (e.g., oligonucleotide or cDNA microarray), reverse transcription (RT)-PCR (e.g., quantitative RT-PCR), or reverse transcription followed by sequencing. The TaqMan® assay and the SYBR® Green PCR assay are commonly used real-time PCR techniques. Other assays include the Standardized (Sta) RT-PCR™ (Gene Express, Inc., Toledo, Ohio) and QuantiGene® (Panomics, Inc., Fremont, Calif.). In some embodiments the level of mRNA is measured. In other embodiments, a reporter-based system is used. Assays for activity of a gene product (e.g., enzymatic activity, binding activity) would be selected base on the particular activity of interest. In general, assays could be cell-free or cell-based in various embodiments of the invention.

A wide variety of test compounds can be used in the inventive methods. For example, a test compound can be a small molecule, polypeptide, peptide, nucleic acid, oligonucleotide, lipid, carbohydrate, or hybrid molecule. Compounds can be obtained from natural sources or produced synthetically. Compounds can be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. The library may comprise, e.g., between 100 and 500,000 compounds, or more. Compounds are often arrayed in multiwell plates. They can be dissolved in a solvent (e.g., DMSO) or provided in dry form, e.g., as a powder or solid. Collections of synthetic, semi-synthetic, and/or naturally occurring compounds can be tested. Compound libraries can comprise structurally related, structurally diverse, or structurally unrelated compounds. Compounds may be artificial (having a structure invented by man and not found in nature) or naturally occurring. In some embodiments, a library comprises at least some compounds that have been identified as “hits” or “leads” in other drug discovery programs and/or derivatives thereof. A compound library can comprise natural products and/or compounds generated using non-directed or directed synthetic organic chemistry. Often a compound library is a small molecule library. Other libraries of interest include peptide or peptoid libraries, cDNA libraries, and oligonucleotide libraries. A library can be focused (e.g., composed primarily of compounds having the same core structure, derived from the same precursor, or having at least one biochemical activity in common).

Compound libraries are available from a number of commercial vendors such as Tocris BioScience, Nanosyn, BioFocus, and from government entities. For example, the Molecular Libraries Small Molecule Repository (MLSMR), a component of the U.S. National Institutes of Health (NIH) Molecular Libraries Program is designed to identify, acquire, maintain, and distribute a collection of >300,000 chemically diverse compounds with known and unknown biological activities for use, e.g., in high-throughput screening (HTS) assays (see https://mli.nih.gov/mli/). The NIH Clinical Collection (NCC) is a plated array of approximately 450 small molecules that have a history of use in human clinical trials. These compounds are highly drug-like with known safety profiles. The NCC collection is arrayed in six 96-well plates. 50 μl of each compound is supplied, as an approximately 10 mM solution in 100% DMSO. In some embodiments, a collection of compounds comprising “approved human drugs” is tested. An “approved human drug” is a compound that has been approved for use in treating humans by a government regulatory agency such as the US Food and Drug Administration, European Medicines Evaluation Agency, or a similar agency responsible for evaluating at least the safety of therapeutic agents prior to allowing them to be marketed. The test compound may be, e.g., an antineoplastic, antibacterial, antiviral, antifungal, antiprotozoal, antiparasitic, antidepressant, antipsychotic, anesthetic, antianginal, antihypertensive, antiarrhythmic, antiinflammatory, analgesic, antithrombotic, antiemetic, immunomodulator, antidiabetic, lipid- or cholesterol-lowering (e.g., statin), anticonvulsant, anticoagulant, antianxiety, hypnotic (sleep-inducing), hormonal, or anti-hormonal drug, etc. In some embodiments, a compound is one that has undergone at least some preclinical or clinical development or has been determined or predicted to have “drug-like” properties. For example, the test compound may have completed a Phase I trial or at least a preclinical study in non-human animals and shown evidence of safety and tolerability. In some embodiments, a test compound is substantially non-toxic to cells of an organism to which the compound may be administered or cells in which the compound may be tested, at the concentration to be used or, in some embodiments, at concentrations up to 10-fold, 100-fold, or 1,000-fold higher than the concentration to be used. For example, there may be no statistically significant effect on cell viability and/or proliferation, or the reduction in viability or proliferation can be no more than 1%, 5%, or 10% in various embodiments. Cytotoxicity and/or effect on cell proliferation can be assessed using any of a variety of assays (some of which are mentioned above). In some embodiments, a test compound is not a compound that is found in a cell culture medium known or used in the art, e.g., culture medium suitable for culturing vertebrate, e.g., mammalian cells or, if the test compound is a compound that is found in a cell culture medium known or used in the art, the test compound is used at a different, e.g., higher, concentration when used in a method of the present invention.

In some embodiments, a method of identifying compounds is performed using a high throughput screen (HTS). A high throughput screen can utilize cell-free or cell-based assays. High throughput screens often involve testing large numbers of compounds with high efficiency, e.g., in parallel. For example, tens or hundreds of thousands of compounds can be routinely screened in short periods of time, e.g., hours to days. Often such screening is performed in multiwell plates containing, e.g., e.g., 96, 384, 1536, 3456, or more wells (sometimes referred to as microwell or microtiter plates or dishes) or other vessels in which multiple physically separated cavities are present in a substrate. High throughput screens can involve use of automation, e.g., for liquid handling, imaging, data acquisition and processing, etc. Without limiting the invention in any way, certain general principles and techniques that may be applied in embodiments of a HTS of the present invention are described in Macarrón R & Hertzberg R P. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Exemplary methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg Hüser.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, cell, or non-human transgenic animal, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1

### Recoding by Multiple Mechanisms Produces Cyclooxygenase and Cyclooxygenase-Related Proteins from Frameshift-Containing COX-3/COX-1b Transcripts in Rat and Human

Cyclooxygenase isozymes catalyze the rate-limiting step in prostaglandin synthesis, are involved in myriad physiological and pathophysiological processes, and are inhibited by aspirin-like drugs.

We previously identified an alternatively spliced, intron-1 retaining variant of COX-1 cloned from canine brain tissue. This new variant, termed COX-3 or COX-1b, is an enzymatically active prostaglandin synthase in dog brain and possesses distinct pharmacological properties relative to COX-1 and COX-2. The COX-3 mRNA is expressed at relatively high levels in a tissue and cell type dependant manner in all species examined. However, in humans and most rodent species, intron-1 is 94 and 98 nucleotides long respectively. Retention of the intron in these species introduces a frameshift predicted to yield a very small 8-16 kD protein with little similarity to either 72 kD COX-1 or COX-2.

Here, we report cloning and ectopically expressing a complete and accurate COX-3 cDNA from both rat and human. Contrary to the small protein predicted from the scanning model of translation, we found that COX-3 mRNA encodes multiple large molecular weight cyclooxygenase-like proteins from the same reading frame as COX-1. Translation of these proteins relies on several recoding mechanisms including cap-independent translation initiation, alternative start site selection, and ribosomal frameshifting. Two COX-3 encoded proteins are active prostaglandin synthase enzymes with activities similar to COX-1 and represent novel targets of NSAIDs. Other COX-3 proteins have unknown functions, but their size and cellular location suggest potential roles as cytosolic enzymes and nuclear factors. Using siRNA and Western blotting we have identified some of these proteins expressed in vivo in multiple tissues and cells.

### BACKGROUND

Cyclooxygenase (COX) is a signaling enzyme that catalyzes the first step of prostaglandins synthesis and is involved in a wide range of processes including inflammation, nociception, thermoregulation, parturition, thrombosis, and cancer(1-6). As their names suggest COX-1 and COX-2 were the first COX isoforms identified. COX-1 is generally regarded as a housekeeping gene and is expressed at constant levels in many tissues. COX-2, on the other hand, shows low constitutive expression and is markedly upregulated during inflammation and a number of other conditions(6,7).

In 2002 a COX-1 alternative splice variant, known as COX-3 or COX-1b, was discovered by our laboratory in large part because it is highly expressed in canine brain tissue(8). This variant retains intron-1 (90 bp long) in its fully processed transcript, inserting 30 amino acids into the N-terminal signal peptide region of the protein. Expression of this variant in Sf9 cells produced an active prostaglandin synthase enzyme that retains a signal peptide and has modified enzymatic activity compared to COX-1.

Northern blot analysis of multiple human tissues found significant levels of COX-3 mRNA in humans with highest expression in cerebral cortex, heart, and skeletal muscle(8). However, intron-1 in human and most rodent species is out-of-frame. Intron-1 in human and rat is 94 and 98 base pairs long, respectively, and introduces a translational reading frameshift beginning at exon-2. Uncorrected, the scanning model of translation predicts the synthesis of a small (8 to 13 kD) protein with little similarity to COX-1.

Some previous studies by others have supported this conclusion; however, these were limited in their ability to investigate whether a complete COX-3 mRNA made only the small predicted protein; or, conversely made high molecular weight proteins because they did not clone, characterize and express the entire COX-3 mRNA. In some cases they used clones that only contained a truncated portion of the putative coding region(9,10). Other studies have indicated that at least one high molecular weight protein is synthesized by RNA editing to correct the frameshift(11). We, however, have been unable to detect these edited transcripts (unpublished data).

Knowledge of the mechanisms behind eukaryotic translation initiation has expanded over the past decade and a number of exceptions to the standard scanning model of translation are now documented (12-15) (13,16-18). We, therefore, hypothesized that recoding mechanisms such as ribosomal frameshifting or alternative translational initiation could function to rectify the frameshift in COX-3 to produce fill-length cyclooxygenase-like proteins in vivo(8) which are unique cyclooxygenase enzymes or related proteins, functional in physiology and disease and potentially novel therapeutic targets.

Here we report the cloning and expression of accurate and complete rat and human COX-3 cDNAs from transient and stable transfections in multiple cell lines and demonstrate that rat COX-3 mRNA makes a minimum of 7 higher molecular weight proteins by utilizing three distinct recoding mechanisms. Two of these proteins, each around 72 kD in size, are N-glycosylated similar to COX-1 and are enzymatically active. Expression of human COX-3 eDNA produced similar results demonstrating evolutionary conservation of these recoding mechanisms in producing proteins from the COX-3 transcript. Analysis of rat tissues as well as multiple human cell lines by a variety of methods including immunoblotting and siRNA knock-down experiments confirmed cellular expression of COX-3 encoded proteins in vivo.

### Results

Polysome Analysis of COX-3 mRNA

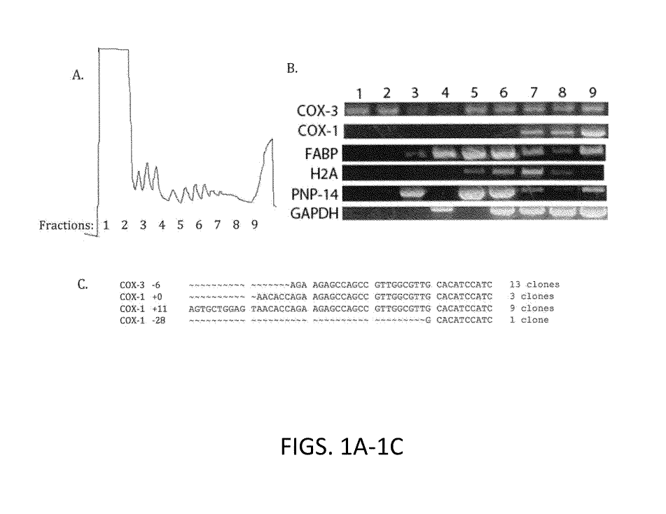

To determine whether COX-3 mRNA is actively translated in vivo, rat spleen polysomes were sedimented through a linear sucrose gradient. Analysis by RT-PCR of each gradient fraction demonstrated that COX-3 mRNA is translated in a complex fashion. Some COX-3 mRNA is translated on heavy polysomes, similar to COX-1 and glyceraldehyde dehydrogenase (GAPDH) mRNAs, each of which have long, actively translated open-reading frames (FIGS. 1A and 1B). Other COX-3 mRNAs are translated on monosomes or with only a few ribosomes similar to short reading frame mRNAs fatty-acid binding protein-7(FABP), phospho-neuro protein-14 (PNP-14), and histone protein 2a (Ht2A).

Cloning of full-length rat COX-3 eDNA

In order to ascertain how COX-3 might be translated on heavy polysomes in vivo, we cloned from this fraction an accurate, full-length rat COX-3 cDNA clone including 5′ and 3′ untranslated regions (UTRs). Analysis by 5′ rapid amplification of cDNA ends (RACE) demonstrated that rat COX-3 utilizes a distinct 5′ transcriptional initiation site 6 base-pairs downstream from the NCBI GenBank (NM_{—}017043) reported +0 cap site of COX-1. This was seen in 13 out of 13 independent COX-3 clones (FIG. 1C). This same analysis of rat COX-1 identified two additional unreported transcriptional start sites, +11 (9 out of 13 clones) and −28 (1 out of 13 clones) with respect to the GenBank consensus 5′ cap site (3 out of 13 clones).

Analysis by 3′RACE demonstrated alternative polyadenylation of the COX-3 transcript relative to COX-1. The first consensus polyadenylation site in COX-1, AAUAAA, is skipped over and an additional ˜2 kB of 3′ UTR information is added in COX-3 mRNA to form a 4.5 kb messenger transcript compared to the 2.8 kb COX-1 transcript.

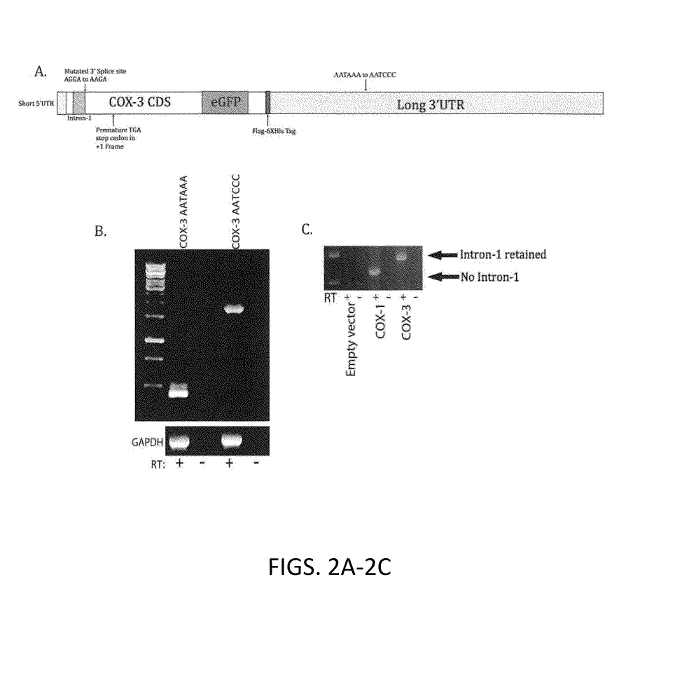

Postulating that the unique UTR regions may regulate translation of the COX-3 mRNA, we cloned the full length COX-3 cDNA with short 5′UTR and long 3′UTR into a pcDNA3.1 mammalian expression vector (FIG. 2A). To ensure expression of authentic COX-3 mRNA, site-directed mutagenesis was used to introduce two changes to the COX-3 cDNA clone. The first was mutation of the consensus 3′ splice acceptor site AGGA to AAGA, a silent mutation, to prevent splicing of intron-1 upon COX-3 expression. Next we mutated the proximal AATAAA consensus polyadenylation signal to AATCCC to prevent recognition of this site by the polyadenylation complex and ensure that the longer 3′ UTR was included on the fully processed COX-3 mRNA.

RT-PCR analysis and DNA sequencing of amplicons corresponding to the 5′ region of the COX-3 mRNA produced by transfection of the COX-3 expression construct into a CHO cell line confirmed that intron-1 is retained in the mature transcripts (FIG. 2C). Additionally, 3′RACE analysis confirmed that mutation of the proximal polyadenylation signal caused polyadenylation solely at the correct location producing an accurate 4.5 kb COX-3 mRNA transcript (FIG. 2B). To facilitate detection and purification of any high molecular weight COX-3 proteins encoded by the COX-3 cDNA we added enhanced green fluorescent protein (eGFP) as well as Flag and His tags to certain COX-3 expression constructs (FIG. 2A). eGFP was inserted between amino acids 500 and 501 while Flag and His tags were placed at the C-terminal end of the coding sequence in-frame with the COX-1(+0) reading frame. In this way, the tags will only be present if the frameshift is rectified or avoided in some manner to produce proteins using the same reading frame as COX-1.

### Detection of High Molecular Weight COX-3 Proteins

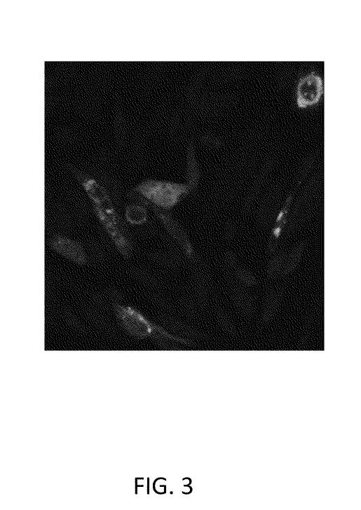

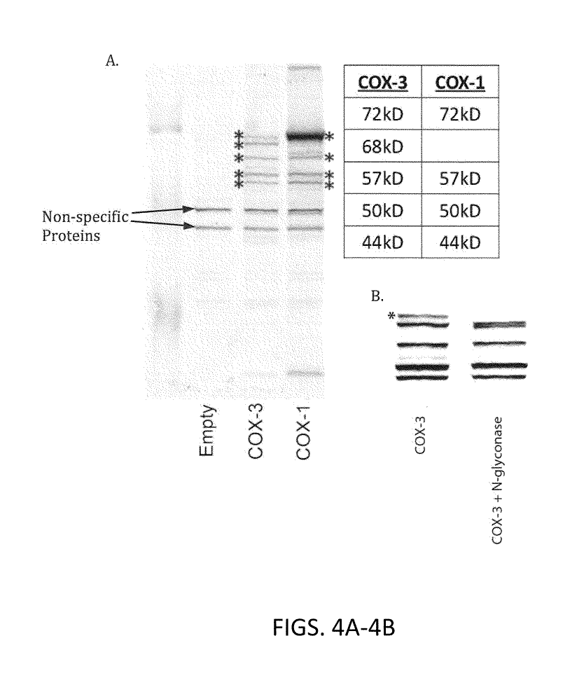

Upon expression of the COX-3 construct and subsequent analysis by fluorescence microscopy, we detected a high percentage of GFP positive cells, confirming that one or more mechanisms result in translation of COX-3 from its long open reading frame (FIG. 3). Immunoblot analysis of these cells using an anti-Flag antibody detected a series of five COX-3 proteins of approximately 72, 68, 57, 50 and 44 kD in size (after subtracting the 27 kD contributed by GFP insertion). The largest of these, the 72 kD COX-3 protein, migrates at the same molecular weight as fully processed and glycosylated COX-1. Cells transfected with a COX-1 construct express 57, 50, and 44 kD proteins in addition to the full-length 72 kD COX-1 form (FIG. 4A).

Both COX-1 and COX-2 are glycosylated with N-linked, high-mannose oligosaccharides on three and four asparagine residues, respectively(6). To determine whether any COX-3 protein(s) are glycosylated, we treated CHO cell lysates with endoglycosidase F (N-glycanase) which caused an increase in the electrophoretic mobility of the 72 kD COX-3 protein, indicating that it is glycosylated in a manner similar to COX-1 (FIG. 4B). However, N-glycanase treatment had no effect on the 68, 57, SO and 44 kD COX-3 proteins, indicating that they are not glycosylated, are not processed through the ER-Golgi and, therefore, are not degradation products of the larger 72 kD COX-3 form, but distinct translation products.

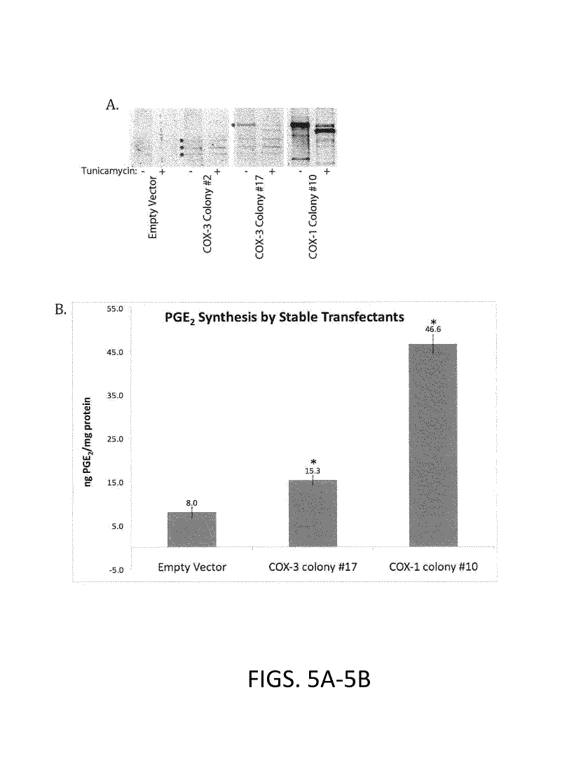

Stable transfectants were created in order to determine whether high-level expression of COX-3 mRNA in transient transfection was artifactually causing expression of these five COX-3 proteins (due to potentially high plasmid copy numbers in individual cells). For this, a clone without the GFP insertion but retaining the Flag and His tags was used. Investigation of individual stable transfectants was done on 24 separate colonies. Two clonally-isolated stable COX-3 colonies showed expression of COX-3 proteins. Interestingly, only one colony (#17) showed expression of the 72 kD COX-3 protein while both expressed the non-glycosylated 57, SO, and 44 kD proteins indicating that not all cells contain the ability to translate the 72 kD form of COX-3 (FIG. 5A). Another indication that cell-specific factors, signals, or mechanisms are required for expression of some COX-3 encoded proteins is that neither COX-3 stable transfectant expressed the 68 kD protein. Expression of this form, like the 72 kD protein, may require clonal isolation of a cell capable of expressing it. As was seen with the transient transfectants, the 72 kD protein was sensitive to tunicamycin, an inhibitor of N-linked glycosylation, whereas the lower molecular weight forms were not.

Three COX-1 colonies showed significant levels of COX-1 protein expression. These colonies homogenously produced only a 72 kD protein and none of the lower 68, 57, 50 or 44 kD proteins.

Stable transfectants were tested for cyclooxygenase activity by anti-PGE₂ radioimmunoassay. As shown, COX-3 colony #17, expressing the 72 kD COX-3 form, showed a statistically significant 91% increase in COX activity over the background level of PGE₂ produced in cells expressing empty vector, confirming that COX-3 encodes an enzymatically active cyclooxygenase enzyme (FIG. 5B).

### Analysis of Recoding Mechanisms for 72 kD COX-3 Protein Expression

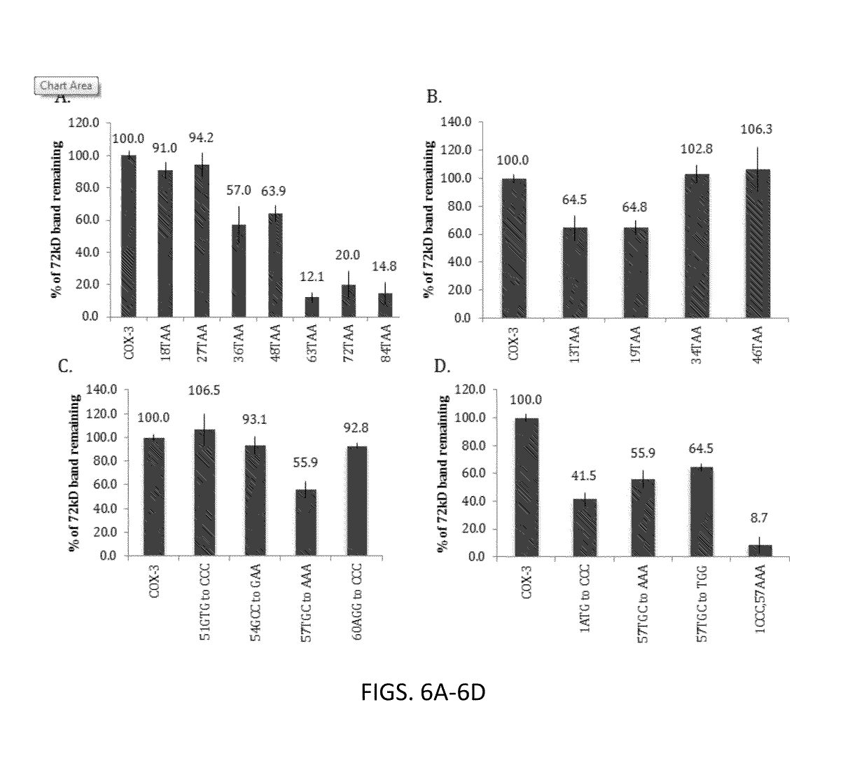

We used site-directed mutagenesis to identify mechanism(s) producing high molecular weight COX-3 encoded proteins. To test for translation initiation at the consensus COX-1ATG followed subsequently by a ribosomal frameshift we mutated ATG-47 to a non-initiating CCC codon. This resulted in a −50% decrease in the expression level of the 72 kD COX-3 protein as determined by immunoblot band intensity (FIG. 6D) indicating that a portion of the 72 kD form initiates on this out of frame ATG codon. Stop TAA codons introduced at different locations of the coding sequence in the +1 reading frame (relative to the COX-1 long open reading frame) showed that positions 59 and 65 bp (nucleotide numbering relative to the COX-3 mRNA 5′ cap site identified earlier) eliminated expression of ˜50% of the protein whereas stop codons further downstream in this same +1 reading frame, at 85 and 92 bp, had no effect on translation levels (FIG. 6A).

Concordantly, TAA stop codons introduced in +0 reading frame at positions 64 and 73 had no effect on expression, whereas stop codons further downstream at positions 82 and 94 bp in the +0 reading frame eliminated −50% of the expression of the 72 kD COX-3 protein (FIG. 6B). Together these results are consistent with 50% of the 72 kd protein translated by ribosomes initialing at ATG 47 (in the +treading frame), translating to a point between nucleotides 73 and 80 (sequence of CCCCAC) and performing a −1 frameshift, placing it into the correct +0 reading frame.

Expression of the remaining 50% of the 72 kD protein was eliminated by introduction of TAA stop codons at position 109, 118, and 130 (FIG. 6A) consistent with a second translation initiation site between nucleotides 94 and 109. Point mutation of each codon between these two nucleotides demonstrated that only one mutation, 103TGC to AAA or TGG, consistently and significantly (−50%) decreased expression of the 72k0 protein indicating that the remaining −50% of the 72 kD COX-3 protein is translated by initiation on this in-frame cysteine codon (FIG. 6C).

As confirmation, a double mutant, 47ATG to GCG (preventing initiation of the frameshifted form) and 103TGC to AAA (preventing initiation of the cysteine form), blocked translation of essentially all of the 72 kD protein (FIG. 6D).

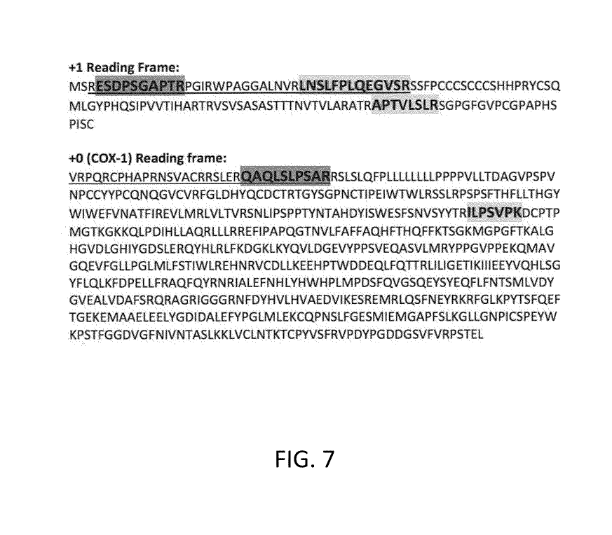

Treatment with N-glycanase consistently caused a mobility shift for about 90% of the 72 kD protein(s), suggesting that both codon 47 and codon 103-initiated proteins contained functional N-terminal signal peptides that directed them into the ER lumen for N-linked glycosylation. The remaining 10% of N-glycanase resistant, non-glycosylated 72 kD COX-3 protein suggested that yet a third 72 kD protein was encoded by the COX-3 mRNA. Its size suggested translation initiates at or near the ATG located at nucleotide 47 in the +1 reading frame used to translate COX-1, To test this hypothesis, AQUA peptide coupled mass spectrometry was performed on trypsin-digested proteins of 50-80 kD expressed by CHO cells transfected with a COX-3 expression construct following Flag and His column purification. Positive identification of two peptides encoded by nucleotides in the +1 reading frame confirm translation of a protein in the +1 reading frame past nucleotides 76 (the point where the glycosylated 72 kD COX-3 protein frameshifted). Detection of this second peptide in the +1 reading frame, located 19 amino acids upstream of the premature stop codon, indicates that the tunicamycin insensitive form is derived from a protein which translates in the +1 reading frame then performs a −1 ribosomal frameshift at some point in the last 19 codons of the +1 open reading frame (FIG. 7).

### Analysis of Recoding Mechanisms for 68 kD COX-3 Protein Expression

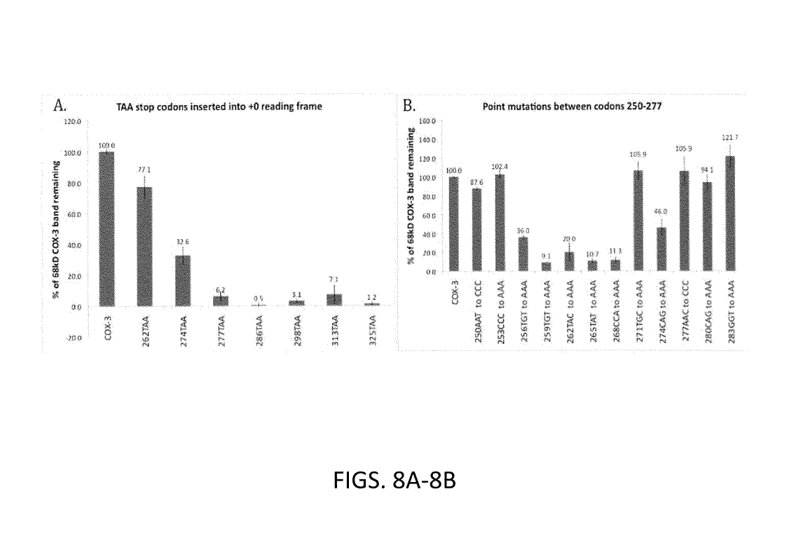

To identify the region of translation initiation for the 68 kD COX-3 form, stop codons were introduced at various positions in the predicted region of initiation (based upon the size of the protein). TAA stop codons at nucleotide 262 had no effect on the translation, whereas stop codons further downstream at positions 286, 298, and 313 completely eliminated expression of the 68 kD protein (FIG. 8A) indicating that translation initiates at some point after position 262, but before 286.

To verify this, we created a series of point mutations of each codon between 250 and 283 and demonstrated that mutation of any codon between 256 and 268 or codon 274 significantly decreased or eliminated translation of the 68 kD protein indicating that this initiation mechanism is dependent upon a 21-nucleotide upstream element for efficient expression (FIG. 5B).

### Analysis of Recoding Mechanisms for 57, 50, and 44 kD COX-3 Protein Expression

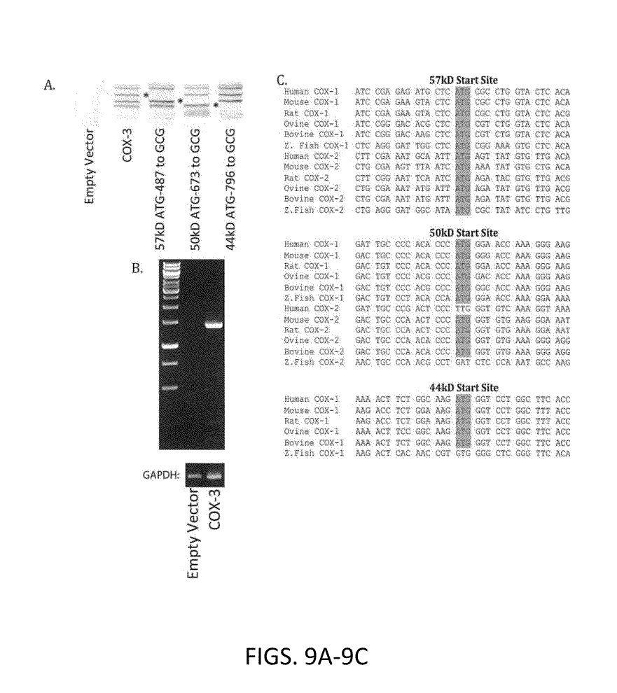

Analysis of the COX-3 nucleotide sequence identified three highly conserved downstream in-frame ATG codons at positions 487, 637, and 796 that could serve as initiation sites to produce the 57, 50, and 44 kD proteins through internal translational initiation (FIG. 9C). Mutation of each of these ATG codons to non-initiating GCG(ala) codons prevented translation of the 57, 50 and 44k1) forms, respectively (FIG. 9A) confirming that these forms are translated through internal translation initiation.

We recognized the possibility that these lower molecular weight forms could be the result of translation from 5′ truncated COX-3 mRNAs produced through cryptic promoter sites or broken mRNAs. However, multiple lines of evidence confirm that these lower molecular weight proteins are translated from the full-length COX-3 mRNA. First, we performed 5′RACE analysis on the COX-3 mRNA following transient transfection in CHO cells and found only full-length COX-3 transcripts (FIG. 9B). In addition, stable transfectants expressing a much lower level of COX-3 mRNA per cell would be predicted to have a negligible level of broken mRNA, yet these cells still express the lower molecular weight proteins.

Finally, previous mutation studies in which stop codons were inserted into the +1 reading frame in the intron-1 region of the COX-3 mRNA and mutation the first ATG codon (47ATG) to CCC resulted in a 3 to 5 fold increase in the expression of the 57, 50 and 44 kD forms suggesting a translational connection between the upstream open reading frame and internal initiation for these lower molecular weight COX-3 proteins.

### Expression and Pharmacological Analysis of Each COX-3 Form

To better understand the unique role of each high-molecular weight COX-3 encoded protein we prepared a series of clones designed to express primarily each COX-3 form. For the 72 kD frameshifted form we artificially corrected the reading-frame by inserting an additional cytosine at position 76 (CCC to CCCC), the identified frameshift site. This clone efficiently expressed a 72 kD glycosylated protein of the same apparent molecular weight as that produced by the authentic COX-3 clone. In addition, we created a clone that efficiently expresses a modified version of the 72 kD cysteine-initiated form by mutating an upstream ATG at position 47 bp to a GCG to prevent any unwanted translation from this codon and replacing the initiating TGC codon at position 103 with an ATG codon. This clone also produced a 72 kD glycosylated protein of the same molecular weight as the authentic 72 kD COX-3 protein.

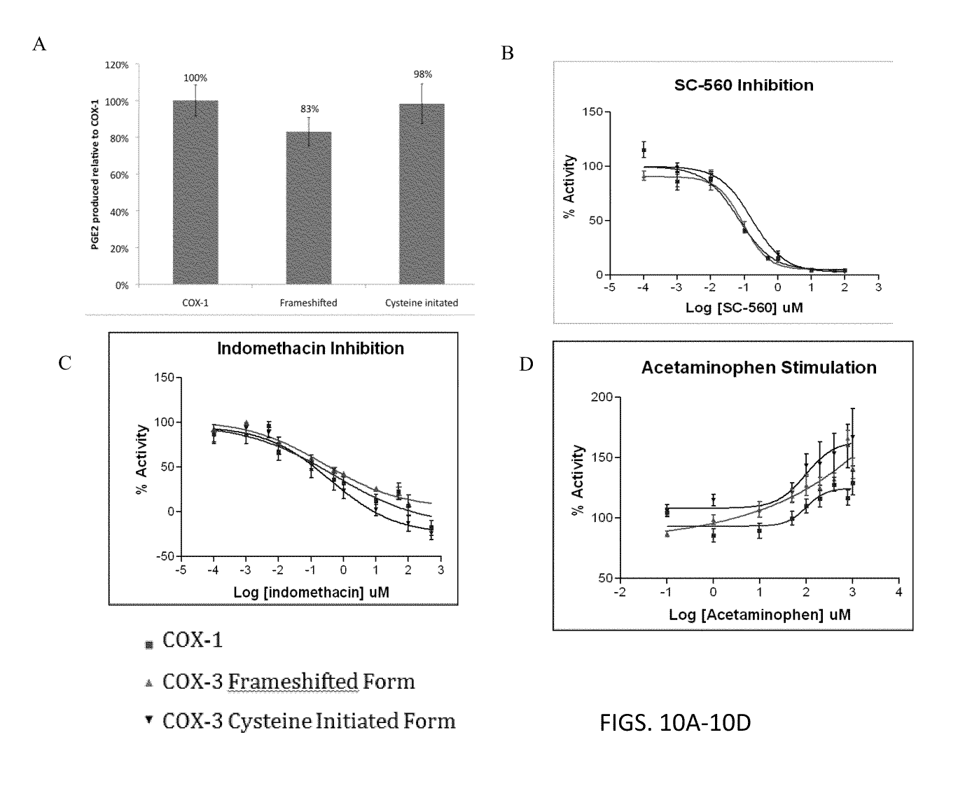

Enzymatic and pharmacologic assays demonstrated that these two 72 kD COX-3 proteins are active prostaglandin synthases and targets of NSAIDs. Both COX-3 proteins showed a high level of cyclooxygenase activity with a specific activity near COX-1 (FIG. 7A). Indomethacin and the COX-1 specific inhibitor SC-560 inhibited both 72 kD COX-3 forms with IC₅₀ values near that seen for COX-1. Additionally, treating these COX-3 forms with the analgesic/antipyretic drug acetaminophen stimulated COX activity with an ECs near that of COX-1 (FIGS. 10B, 10C and 10D).

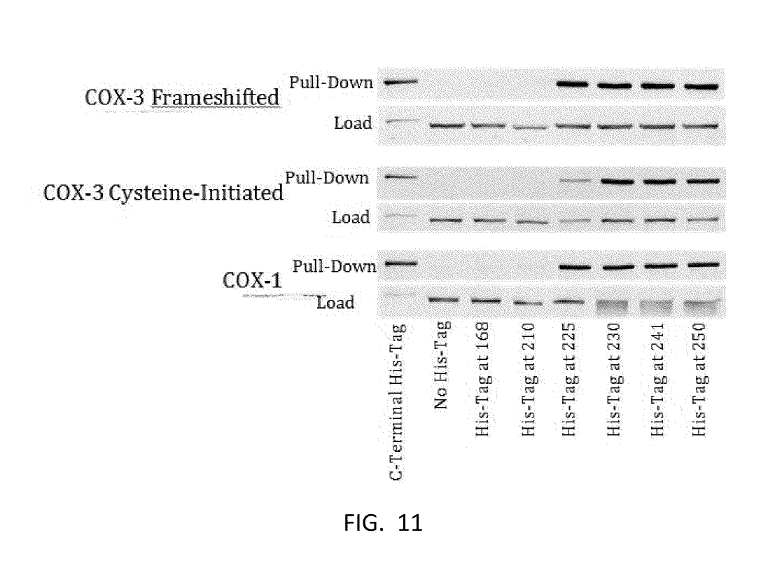

Given that both of these COX-3 proteins have an electrophoretic mobility very close to that of fully processed COX-1, we sought to determine whether the signal peptide for these COX-3 clones is cleaved as is the case for COX-1. To do this we engineered and expressed a series of clones containing His tags placed at different locations along the N-terminal region of the proteins and tested for the presence of the His tag by purification over a cobalt resin column. The results presented in FIG. 11 show that when expressed in CHO cells, the signal peptide is cleaved from both the frameshifted and cysteine-initiated forms of COX-3 in a region very close to that of COX-1 with the cysteine initiated form cleaving one or two amino acids downstream from the COX-1 cleavage site.

In order to better analyze the 57 kD, 50 kD, and 44 kD forms, we engineered clones truncated to remove sequence upstream of each initiating ATG codon. For the 57 kD form we additionally mutated the 50 kD and 44 kD AT codons to GCG codons to prevent simultaneous expression of these forms. In the case of the 50 kD clone, we also mutated the 44 kD ATG to GCG for the same reason.

CHO cells transiently expressing each of these clones were assayed for COX activity, but showed no significant difference when compared with cells expressing an empty vector control (data not shown).

### Expression of Human COX-3

Having confirmed that the rat COX-3 message encodes multiple large molecular weight proteins we sought to determine whether the same was true of human COX-3, which also exhibits a frameshift due to an intron-1 length of 94 bp. To do this we cloned the entire human COX-3 cDNA into a pcDNA 3.1 mammalian expression vector, mutated the 3′ splice acceptor site to prevent splicing out of intron-1, and mutated the proximal polyadenylation signal to ensure inclusion of the longer 3′UTR in the final message, as we did for rat COX-3. We also placed Flag and His tags on the (−terminus of the coding region to facilitate detection and purification of human COX-3 encoded proteins.

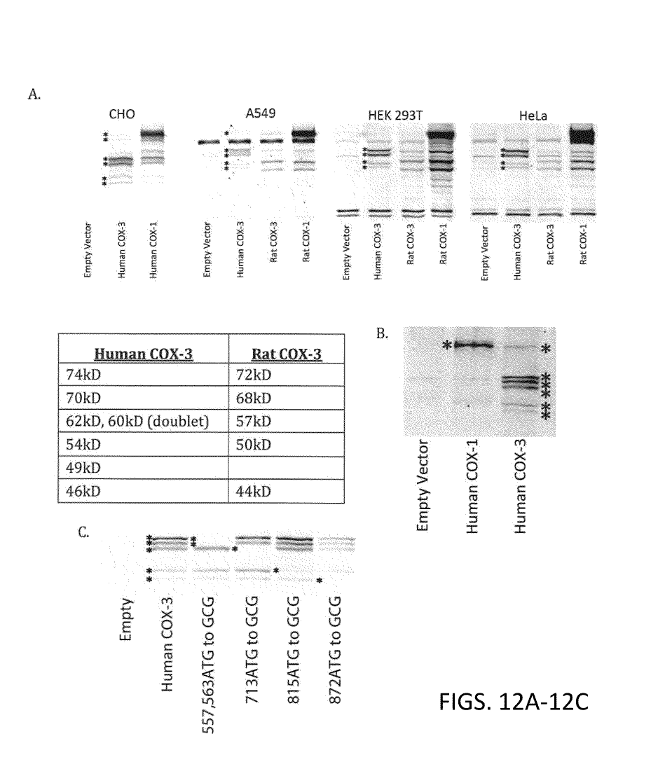

Transient expression of this hCOX-3 clone in different cell lines confirmed that human COX-3 mRNA also encodes multiple high molecular weight proteins similar to those seen for rat COX-3 (FIG. 12A). Seven proteins are detected in CHO and A549 cells with molecular weights of 74, 70, 62, 60, 54, 49, and 46 kD. However, when expressed in HeLa and 293 cells the largest forms are not expressed, again indicating that expression of the largest of these proteins relies upon processes expressed in a cell-type dependent manner.

As a test for over-expression artifacts, we generated A549 cells stably expressing our human COX-3 construct and verified expression of the same proteins by anti-Flag Western blot (FIG. 12B).

As predicted by the evolutionary conservation of the ATG internal initiation codons identified in rat COX-3, site-directed mutagenesis of their human analogs, ATG codons at positions 557&563, 713, and 872 blocked expression of the 62&60 kD, 54, and 46 kD forms respectively (FIG. 12C). The human COX-3 mRNA contains an additional ATG codon at position 815 which is not found in the rat mRNA. Mutation of this ATG to GCG blocked expression of the 49 kD COX-3 protein.

We note that the 62&60 kD forms appear to be alternatively modified versions of the same protein as mutation of a single codon, ATG-557, reduced expression of both forms by −80%, while mutation of an ATG codon just 3 nucleotides further downstream at 563 (conserved in rat) also reduced expression of both forms, but by only −20%. Mutation of both codons completely eliminated expression of this doublet indicating that either site can be used, but that ATG-557 is preferentially used for initiation.

### In Vivo Identification of COX-3 Encoded Proteins

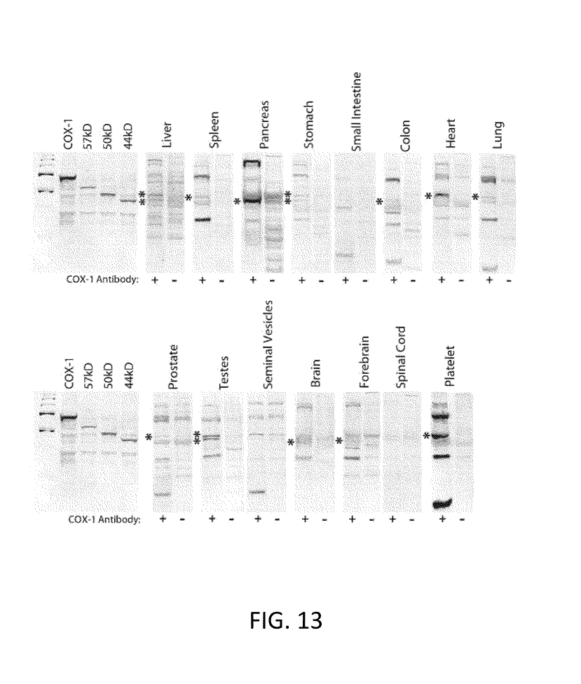

Having defined the proteins encoded by both human and rat COX-3 mRNAs, we tested whether any of these COX-3 proteins are expressed in vivo. To do this, we screened rat tissues by Western blot for expression of the lower molecular weight forms of COX-3. As shown in FIG. 13 we detected modest levels of both 50 kD and 44 kD COX-3 proteins in multiple rat tissues with the highest levels seen in platelet, pancreas, testes and heart. These were shown to be N-glycanase insensitive, a signature of SOkD and 44 kD COX-3 encoded proteins, but not of degradation products of COX-1.

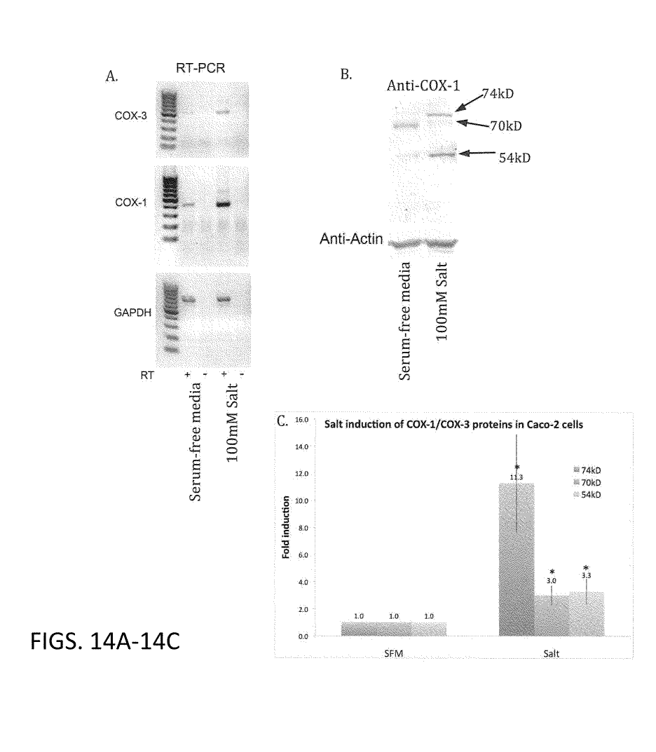

To study the in vivo expression of human COX-3, we used a COX-3 induction model developed by Nurmi et. al.(19) We treated Caco-2 cells with 100 mM NaCl for 22 hours and detected a large induction of COX-1 and more modest induction of COX-3 mRNA by RT-PCR analysis (FIG. 14A). Immunoblots demonstrated a concomitant 11 fold increase in a 74 kD protein (expected size of COX-1) as well as a smaller ˜3 fold increase in 70 kD, and 54 kD proteins (FIGS. 14B and 14C). Both correspond in size with the human 70 kD (analog of the rat 68 kD) and the 54 kD (analog of the rat 50 kD) COX-3 proteins identified in ectopic expression studies.

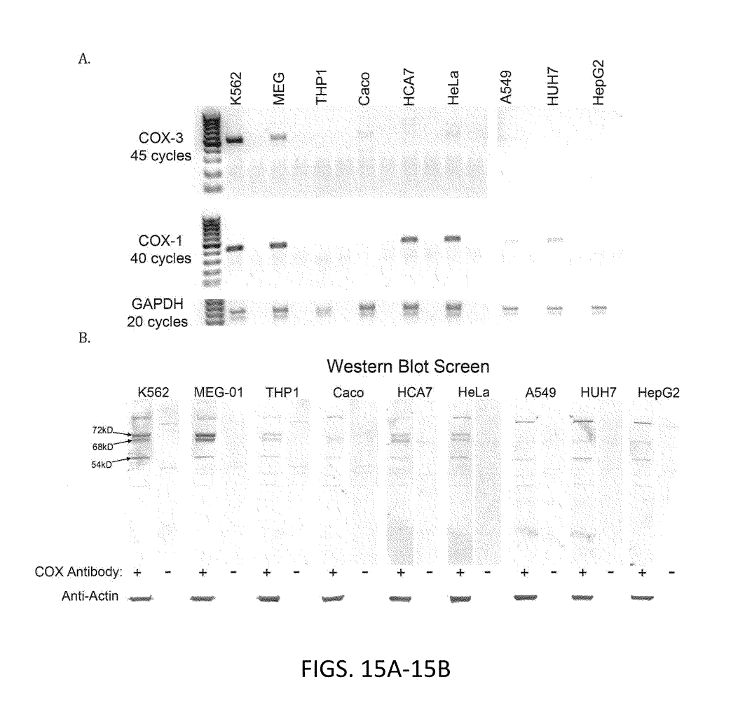

We then screened 8 additional human cell lines for COX-3 expression by RT-PCR and by Western blot. As shown in FIG. 15A, COX-3 mRNA is expressed widely in the majority of the cell types, with highest expression in KS62 cells and MEG-01 cells, Immunoblot analysis detected 74 kD, 70 kD and 54 kD immunoreactive proteins (FIG. 15B) in many of these cell lines. The expression of the 74 kD protein correlated generally well with the level of COX-1 mRNA while the 70 kd and 54 kD proteins correlated generally well with COX-3 mRNA expression.

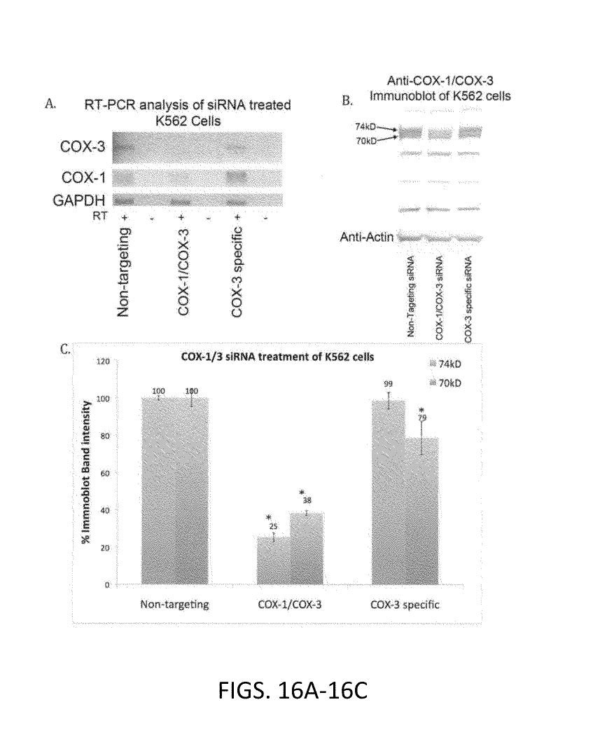

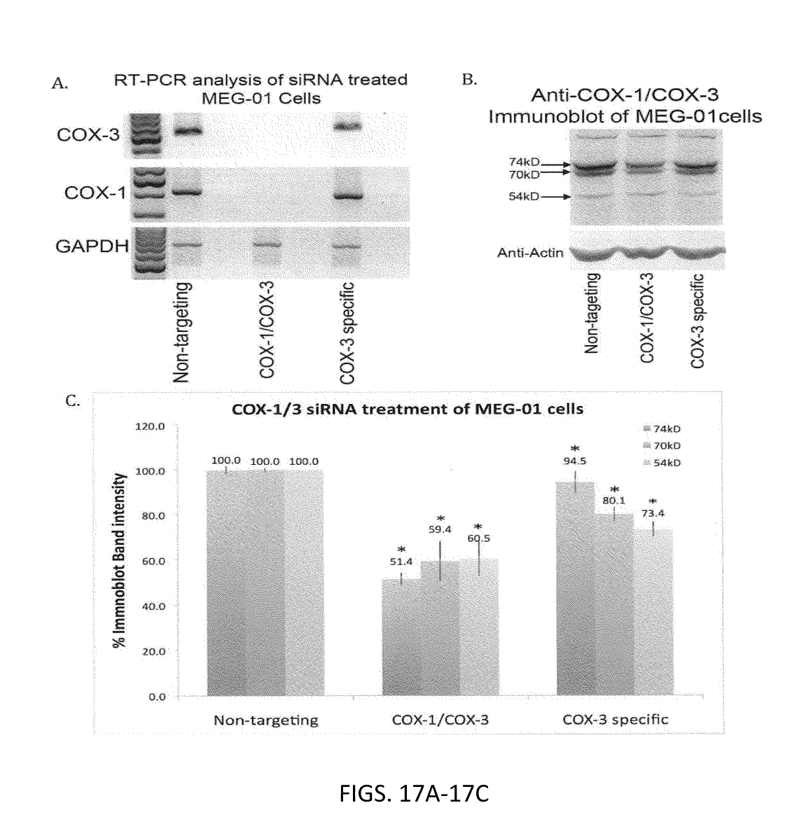

To confirm that the lower molecular weight 70 and 54 kD) forms were derived from the COX-3 mRNA, we treated both KS62 and MFG-01 cells with siRNAs targeting either exons 10 and 11 of the COX-1/COX-3 mRNAs or intron-1 of the COX-3 mRNA. RT-PCR analysis demonstrated that the COX-3 specific (intron-1) siRNAs knock-down approximately 40% of the COX-3 mRNA with no effect on COX-1 mRNA, but that they are not as efficient as exon 10&11 siRNAs which knock-down both COX-1 mRNA and COX-3 mRNA at about 90% efficiency (FIGS. 16A and 17A).

Treatment with COX-3 specific siRNAs lead to a statistically significant-20% decrease in the intensity of the 70 kD protein (26% decrease in 54 kD) in both KS62 and MEG-01 cells with no decrease in the level of the 74 kD COX-1 protein (FIGS. 16B, 16C, 17B and 17C) confirming that the 70 kD) (and 54 kD in MEG-01) proteins are translated from the COX-3 mRNA in vivo in these cell lines. Immunoblotting of exon 10&11 siRNA treated cells detected a statistically significant −50% decrease in the 74 and 70 kD (and 54 kD in MEG-01) proteins indicating these are derived from either the COX-1 or COX-3 transcripts.

### DISCUSSION

In total our data indicates that the COX-3 mRNA is translated through multiple recoding mechanisms to produce at least five cyclooxygenase-related proteins whose expression depends upon specific tissue and cellular conditions. This is consistent with prior reports related to expression of other mRNAs where a single transcript gives rise to multiple versions of a protein by initiating translation at multiple sites (reviewed by Touriol et. al. (20)), Ingolia, et al. (21,22) demonstrated, using ribosomal profiling, that 65% of all transcripts in mouse embryonic stem cells contain more than one translational initiation site that is used at a relatively high level, while 16% contained four or more start sites producing either N-terminal extended or N-terminal truncated proteins(22).

More recently, Lee et. al. using a modified ribosome profiling method analyzed initiation sites for a large number of mRNAs in mouse embryonic fibroblast cells. While the overall signal for COX-1 mRNA is low, they were able to identify four previously unreported downstream translation initiation sites for COX-1 in addition to the annotated ATG codon(23) confirming that this mRNA is heavily recoded. One of the initiation sites they identified in these mouse cells (155 bp) is very close to the position of the initiation site we identified for the 68 kD form of rat COX-3 mRNA.

The precise mechanism by which internal translation start sites are selected, in light of the scanning model, is not yet well understood, but likely involves one of two processes. The first, leaky scanning, requires the ribosome to scan from the 5′ cap site and skip over the first ATG codon to initiate at a down-stream ATG codon(18,24). Often this occurs when the nucleotides surrounding the first ATG codon (known as the Kozak consensus sequence) are sub-optimal. The second mechanism relies on cap-independent translation. In this process the ribosome is recruited directly to internal portions of the mRNA, usually through the action of structurally complex RNA motifs known as internal ribosomal entry sites (IRES) or related RNA sequences known as cap-independent translation enhancer sequences (CITEs)(25). Both contain high-affinity binding sites for either the ribosome itself or eukaryotic initiation factors which lead to internal translation initiation (26).

Our data indicate efficient translation of the 68 kD COX-3 form depends on a ˜22 nucleotide-long region near the initiation site perhaps indicating that it is translated through a cap-independent mechanism. Whether a similar large region is necessary for translation of the 57, 50, and 44 kD COX-3 forms has not been directly assessed, and the mechanism of downstream initiation for this mRNA will require further research. However, this may suggest the lower molecular weight forms of COX-3, produced through initiation at internal codons, play a role in processes such as hypoxia, cancer, viral infection response, and other conditions in which cap-dependant translation is shut down and cap-independent translation predominates(27-29).

Although often associated with prokaryotes, eukaryotic translation initiation can occur at non-ATG codons, and reportedly accounts for as much as 55% of all initiation events(20,22). Near-cognate codons are often used but translation can initiate at other codons as well, such as the initiating TGC codon identified in our work. Recently, Anaganti et. al. reported the cloning of a short CAPC (a tumor suppressor and inhibitor of NF-KB signaling, also known as LRRC26 leucine rich repeat containing 26) variant from human cancer cell lines that lacked any ATG codons(30). Upon expression it was determined, using mass spectrometry, that a 44 amino-acid polypeptide is efficiently expressed through translation initiation at a cysteine encoded by a TGC codon. Analysis of the mRNA sequence at the initiating TGC codon for this S-CPAC variant (GCC TGC CGT) shows some similarity with the start site identified in our study for the 72 kD cysteine-initiated COX-3 form (GCC TGC AGG) perhaps indicating a role for surrounding nucleotides in initiation at TGC or other non-cognate codons.

### Role of COX-3 Proteins

We have identified two separate recoding mechanisms by which cells can produce active prostaglandin synthase enzymes from COX-3 mRNA with activities similar to COX-1. This raises the question of what unique functions the COX-3 enzymes accomplish in cells. The answer may be that, similar to the distinction between COX-1 and COX-2 which are analogous from an enzymatic standpoint, regulation is key. For example, Nurmi et. al. found a rapid induction of COX-3 mRNA in response to osmotic stress while COX-1 is not induced significantly until 20 hours after treatment(19). If this same treatment is performed on cells following siRNA knockdown of COX-3, COX-1 and COX-2 expression is rapidly induced to compensate for the lack of COX-3 expression indicating that, in this model, COX-3 expression is coordinately regulated with COX-1& COX-2 expression with cross-talk between expression of the three isoforms.

The fact that COX-3 mRNA is expressed at significantly high levels in a tissue and cell-specific fashion further suggest a role in physiology. We initially found COX-3 mRNA expressed in specific regions of brain and heart(8). Kis et. al. further looked at expression of COX-3 mRNA in various brain cell populations by RT-PCR and determined that COX-3 mRNA in the brain is strongly expressed in endothelial cells, but at much lower levels in neurons, astrocytes, pericytes and choroidal epithelial cells(31). This is corroborated by Northern blot studies demonstrating the highest levels of COX-3 mRNA expression in highly vascularized tissues including heart, skeletal muscle, placenta, liver, spleen and stomach(11). Taken together these data suggest that COX-3 may play a role in vascular function; perhaps in processes including vasodilatation, clotting, or remodeling.

With regard to acetaminophen, we determined that unlike canine COX-3, which is specifically inhibited by acetaminophen, the 72 kD forms of COX-3 were both stimulated in the presence of micromolar concentrations of acetaminophen in our assay. Stable expression of COX-3 demonstrated that the 72 kD forms of COX-3 require cell-specific conditions to be efficiently expressed. It is likely that in our over-expressed COX-3 assay we are not fully mimicking the cellular conditions required for acetaminophen inhibition, which is highly dependant upon intracellular oxidant tone. This could also represent an authentic difference between canine and rodent COX-3 enzymes. Further research will be needed to fully elucidate the contribution of COX-3 to acetaminophen-induced analgesia and antipyresis.

The functions of the lower molecular weight COX-3 encoded proteins are intriguing because the 68 kD, 57 kD, 50 kD, and 44 kD COX-3 forms are missing signal peptides which should prevent translocation of these proteins to the ER for glycosylation. Glycosylation has previously been shown to be necessary for folding of the COX enzyme to its catalytically active form; however, if the carbohydrate residues are removed post-translationally the enzyme retains peroxidase activity(32). Due to the fact that the entire peroxidase active site region of the protein is still present it is possible that even without glycosylation these proteins fold into active peroxidase enzymes. In fact, the 68 kD protein lacks only the first ˜11 amino acids compared with fully processed COX-1, and retains the entire dimerization, membrane binding, peroxidase and cyclooxygenase active site domains.

Sequence alignment between COX-1 and COX-2 suggest the possibility of recoding of the COX-2 mRNA as well. The details of the mechanisms by which translation, splicing and recoding of COX-3 are coordinated are being further investigated, however, we have previously shown for COX-2 the retention of a portion of intron-1 is exquisitely regulated in response to mitogenic stimulation(7). The final translational product of the intron-1 retained COX-2 mRNA has never been determined due to the presence of multiple stop codons within the intron, but in light of our results showing extensive recoding of intron-1 retained COX-1 (COX-3 mRNA) we propose that a careful analysis of this alternatively spliced mRNA will demonstrate similar recoding mechanism result in translation of COX-2 related proteins from this COX-2 mRNA as well.

Cyclooxygenases function at the heart of critical physiological processes including nociception, pyresis, thrombosis, inflammation, regulation of vascular tone, ovulation, implantation, angiogenesis, parturition and pathophysiological processes such as neoplasia and inflammatory diseases. These results lay a groundwork for additional studies which will further explain how the cyclooxygenase enzyme system regulates aspects of these dynamic processes.

### Materials and Methods

All animal studies were reviewed and approved by the institutional animal care and use committee at Brigham Young University

### Cell Culture

Cell lines used in this study were from ATCC and were used at low passage numbers. Cells were maintained at 37 C and 5% CO₂ and passaged once cells reached-90% confluence. Chinese hamster ovary (CHO), A549, HeLa, HUH7, HepG2, and cells were grown in DMEM/F12 50:50 (Sigma-Aldrich or Gibco) medium with 10% FBS (Sigma-Aldrich) and 1% penicillin/streptomycin (P/S)(Gibco) supplements. K562, MEG-01, and THP1 cells were maintained in RPMI 1640 medium with 10% FBS, 1% P/S, and 1% glutamine added (Gibco). Caco-2 cells were maintained in DMEM, high glucose with 20% FBS, 1% F/S, and 1× non-essential amino acids. Prior to each experiment cells were seeded in fully supplemented standard growth medium without antibiotics. For tunicamycin treatments, 10 μg/mL tunicamycin was incubated with the cells for 24 hours in standard cell culture medium prior to harvesting.

For salt treatment of Caco-2 cells, cells were grown to ˜95% confluence in standard medium then growth medium was replaced with media minus supplements with or without 100 mM NaCl added. Cells were grown for 22 hours at 37° C. then protein and RNA was harvested and analyzed by Western blot and RT-PCR.

### Polysome Analysis

Polysome analysis was performed following the protocol of Zhu et. al.(33) Briefly, −100 day old male Long Evans rats (Charles River) were sacrificed by decapitation and spleens removed and homogenized by polytron into a solution of 1×LSB (20 mM tris, 10 mM NaCl, 30 mM MgClz) containing 100 μg/ml emetine (Sigma), 160 U/ml RNasin (Invitrogen), 10 mM VRC (ribonucleoside vanadyl complex, Sigma), 1 mM DTT (dithiothreitol, Sigma), and 200 μg/ml heparin(Sigma). Igepal CA 630 was added to a final concentration of 1.2% and further homogenized by 10 to 20 strokes in an ice-cold dounce. Nuclei and mitochondria were sedimented by centrifugation and the resulting supernatant mixed 1:1 with polysome sail solution (1.07 M sodium chloride, 7 mg/ml heparin, 0.1 mM DTT, 0.2 U/μL RNasin, 360 μg/ml emetine, and 100 mM VRC).
RNA-containing lysate (200 μL) was layered directly on top of a 4.8 ml 15% to 50% sucrose gradient and centrifuged at 45000 rpm in an SW 55 ti rotor for 90 minutes at 4° C. After centrifugation, ten 0.5 mL fractions were collected while monitoring the 254 nm absorbance using an ISCO-5A spectrophotometer/fractionator. RNA was purified from each fraction using the RNeasy midi RNA purification kit (Qiagen) by mixing gradient fractions with RLT buffer and 1 volume of 70% ethanol and following the recommended protocol. Expression levels of mRNA in each fraction were analyzed by RT-PCR using the following primer pairs for PCR amplification.

COX-3:
GTCATGAGTCGTGAGD(forward)
and
GTACAACTCTCCATCCAGCA(reverse),
COX-1:
CAGAGTCATGAGTCGAAGA(forward)
and
GTACAACTCTCCATCCAGCA(reverse),
GAPDH:
GGTGGAAGAATGGGAGTTG(forward)
and
GGTGGAGAATGGGAGTTG(reverse),
H2A:
CTCGTGCAAAAGCGAAGTCT(forward)
and
TACCCAAGCCTCTCCTCAGA(reverse),
FABP:
AAGGGCAAGGATGGTAGATG(forward)
and
CCTCCACACCAAAGACAAAC(reverse),
PNP-14:
GCAGAGAAGACCAAGGAAGG(forward)
and
CATGCCACAATCACAACGTA(reverse).

5′ and 3′Rapid Amplification of cDNA Ends

5′ and 3′rapid amplification of cDNA ends (RACE) analysis on endogenous rat spleen RNA was performed using the RLM race kit (Ambion). RACE analysis on transiently transfected CHO cells was performed using GeneRacer kit (Invitrogen) following the recommended procedures. Platinum Pfx amplification kit (Invitrogen) was used for PCR reactions. Primers AGACTCCTFCACTCATGACGACTC (COX-1) and ACTCCTTCCTGCAGAGG (COX-3) as well as a sense-strand primer complementary to the 5′ adapter sequence (provided by the manufacturer) were used for amplification of the 5′ end of COX-1 and COX-3. Primers TGTGCCAGATTACCCTGGAGA (COX-1) and GGGAACTGGTTTTCAACTGGAGG (COX-3) and the provided antisense primer complementary to the 3′ adapter sequence were used for amplification of the 3′ end.

Cloning of Rat COX-3 eDNA

The 5′ UTR and the long 3′UTR were cloned by 5′ and 3′RACE respectively as described. In preparing the 5′ end of the COX-3 cDNA, we simultaneously mutated the intron-1 splice acceptor site from AGGA to AAGA in order to prevent splicing out of the intron. The remainder of the COX-3 CDS and the first 2000 bp of the 3′UTR were PCR amplified and cloned into a pcR-4 TOPO vector. Flag and His tags were inserted between the last codon of the CDS and the UAG stop codon in order to facilitate detection of any translation products produced by the COX-3 cDNA These four DNA segments were fused together by fusion PCR amplification to yield a full-length 4.3 kb COX-3 cDNA clone. The full-length COX-3 cDNA was subcloned into a pcDNA 3.1(+)mammalian expression vector (Invitrogen). In an additional construct, enhanced green fluorescent protein (eGFP) was inserted into a ClaI site in COX-3 between amino acids 500 and 501 of COX-3.

Finally, site-directed mutagenesis was used to mutate the first polyadenylation signal sequence at position 2937 bp from AATAAA to AATCCC to prevent premature polyadenylation of encoded transcripts and nucleotides between the vector's transcriptional 5′ cap site and the beginning of the COX-3 insert were removed so that the final COX-3 mRNA would begin at the authentic 5′ end. Human COX-3 expression constructs were made in a similar fashion to the rat COX-3 cDNA constructs.

### Transient Protein Expression

Transient transfection of COX-3 and other constructs into CHO cells was performed using lipofectamine reagent (Invitrogen) following the recommended procedure. HeLa, A549, and 293T cells were transfected using Lipofectamine 200 (Invitrogen) following the recommended protocol.

For immunoblot analysis, cells were scraped up into RIPA buffer (50 mM tris pH 8.0, 150 mM NaCl, 1% NP-40, 0.5% deoxycholate, 0.1% SDS, 1× Protease inhibitor cocktail from Roche) and lysed by passing through a 25-gauge syringe twelve times. Samples were centrifuged in a microfuge at full speed for 5 minutes to sediment insoluble material, protein concentration was determined by DC assay, and samples analyzed by Western blot using nitrocellulose membrane blocked with 2.5% milk.

Primary antibodies used in this study include anti-DDDDK (Flag) (Abeam ab1162 at a 1:1000 dilution), anti-neomycin phosphotransferase (Abcam ab60018 at a 1:1000 dilution), anti-rat COX-1 (Cayman Chemicals—160109 at 1:1000 dilution), anti-human COX-1(Santa Cruz Biotech-1752R at 1:200 dilution), and anti-actin (Abeam ab3280 at 1:7000 dilution). Blots were probed with IR dye labeled secondary anti-rabbit (926-32211) and anti-mouse (926-32220) antibodies (LI-COR Biosciences) and imaged using an Odyssey scanner (LI-COR Biosciences). Odyssey application software was used for all protein quantitation measurements.

Preparation and Analysis q/Stable Transfectants

Stable CHO and A549 transfectants were prepared by first linearizing empty pcDNA 3.1 vector, Flag/His Tagged-COX-1(no GFP), and Flag/His-tagged COX-3 (no GFP) plasmid DNA with Mfel (New England Biolabs) digestion for one hour. DNA was transfected into the cells and after 24 hours, cells were seeded onto a 100 mm plate in standard growth medium containing 750 μg/mL Geneticin (Gibco) and cultured in this selection medium for ˜2 weeks. Single cells were isolated by seeding them out in 96 well plates at a density of ½ cell per well in standard media with geneticin. Individual colonies were screened for COX expression by immunoblot using an anti-flag (Abeam ab1162) and an anti-COX-1 (Cayman Chemicals 160109) antibody and by anti-PGE₂ radioimmunoassay.

### Protein Purification

To enrich ectopically expressed COX-3 encoded protein by immunoprecipitation, cells were scraped into RIPA buffer and passed through a 25-gauge needle 12 times to lyse. Lysates were cleared by centrifuging at 15,000 g for 15 minutes and protein concentration of the supernatant was determined by DC protein assay (Bio-Rad). Anti-flag antibody resin (20 μL/milliliter of lysate, Sigma) was added and incubated overnight at 4° C. on a rotator. In the morning, beads were sedimented by centrifugation at 3000 rpm for 5 minutes and the supernatant removed using a syringe and 25-gauge needle. The beads were washed twice with 5 volumes RIPA buffer and the protein eluted by adding 2 volumes of RIPA buffer containing 300 μg/mL 3× Flag peptide (Sigma) and incubating at 4° C. for one hour on a rotator.

For purification of N-terminal His-tag insertion proteins, carboxy-terminal Flag and His tags were removed from the rat COX-1, frameshift corrected COX-3, and cysteine initiated COX-3 expressing clones. For the purpose of identifying cleavage sites for the signal peptide, His tags were then inserted by site-directed mutagenesis into various positions in the N-terminal region of each clone. The His-tag insertion sites were selected to minimize disruption of stretches of hydrophobic amino-acids and interference with signal recognition particle binding. These clones were then transiently expressed in CHO cells and proteins chromatographed over a cobalt resin column (Thermo Scientific). Protein was eluted with 50!IL elution buffer (500 mM NaCl, 20 mM HEPES pH 7.4, 30 mM β-octyl glucoside, 500 mM imidizole) and analyzed by immunoblot using an anti-COX-1 antibody (Cayman Chemicals 160109).

### Cyclooxygenase Activity Assays

To measure prostaglandin production, CHO cells transfected with COX-1, COX-3, or empty vector constructs were treated with arachidonic acid (5 μM) for 15 minutes at 37° C. and aliquots (100 μL) of media were subsequently assayed for PGE₂ levels via dextran coated charcoal based competitive radioimmunoassay (RIA) using an anti-PGE₂ antibody (Sigma-Aldrich) and tritiated PGE₂ (Perkin Elmer) following the antibody manufacturer's recommended protocol. PGE₂ levels in cells expressing empty vector were used as a standard for background levels of PGE₂. For inhibition studies, cells were pretreated with drug for 30 minutes at 37° C.

Whole cell lysates of COX-3 stable transfectants were analyzed in the same manner, except that cells were first lysed into PBS containing protease inhibitors. Arachidonic acid (30 μM) was added to the lysates to initiate the reaction and samples were incubated for 15 minutes at 37° C. Following the incubation, samples were heat inactivated at 65° C. for 15 minutes. As an additional control to measure background levels of PGE2 in the stable transfectants, an aliquot of each lysate was heat inactivated before being mixed with arachidonic acid, incubated for 15 minutes at 37° C., and assayed by RIA.

### Site-Directed Mutagenesis

All mutagenesis experiments were performed using the Geneart site-directed mutagenesis kit from Invitrogen. A forward primer was prepared which overlapped the mutation site and contained the desired mutation. A reverse primer was also prepared so that there were at least 12 nucleotides of overlap between the 5′ end of the reverse primer and the 5′ end of the forward primer. PCR amplification was performed using platinum Pfx (Invitrogen).

The reaction mixture was subjected to Dpnl digestion overnight followed by in vitro recombination to circularize the amplicon. DH5a chemically competent E. Coli (Invitrogen) were transformed with the circularized amplicon, single colonies inoculated into ampicillin-containing LB broth, cultured overnight at 37° C., and plasmid DNA purified and sequenced by dideoxy BigDye based sequencing (BYU DNA sequencing center).

### Mass Spectrometry

COX-3 was transiently expressed in CHO cells and protein purified by anti-flag immunoprecipitation followed by chromatograghing through a cobalt resin column. Fluted protein was desalted and concentrated by centrifuging through a 30 kD cutoff ultrafiltration column then electrophoresed through a 10% acrylamide gel. Proteins were stained with coomassie blue (Pierce) and a gel slice encompassing proteins from 50 kD to 80 kD in size was excised and analyzed via AQUA-peptide assisted mass spectrometry analysis.

### Rat Tissue COX-1 and COX-3 Screen

A male Long Evans rat, ˜200 days old, was anesthetized and sacrificed by decapitiation. Organs and tissues were removed, rinsed in PBS, and polytron homogenized into PBS with protease inhibitor cocktail (Roche) at a ratio of 4 mL per gram of tissue. SDS was added to 0.5% and protein concentration determined by DC assay.

Platelets were isolated by collecting trunk blood into a room temperature solution of PBS with 250 U/mL heparin (Sigma-Aldrich). Blood was centrifuged twice at 500 g for 2 minutes and the supernatant (platelet-rich plasma) removed and then centrifuged at 12,000 g for 5 minutes to sediment platelets.

For immunoblots, 30 μg of protein was electrophoresed, transferred to a nitrocellulose membrane, and probed using either an anti-COX-1 antibody (Cayman) or purified non-immune rabbit IgG's at the same 1:1000 dilution. N-glycanase treatment was performed using enzyme purchased From Prozyme following the recommended protocol.

### Analysis of Eel/Lines For COX-1 and COX-3 Expression

For protein analysis, cells were scraped into 1 mL RIPA buffer and lysed by passing through a 25 gauge needle 12 times. Lysate was cleared by centrifuging samples at full speed in a microfuge for 15 minutes at 4° C. and protein concentration determined by DC assay. Protein (30 μg) was analyzed for COX-1/COX-3 expression by Western blot probing with a COX-1 antibody (Santa Cruz Biotech, sc-1752R) raised against an epitope near the (−terminus of human COX-1.

For RT-PCR analysis, RNA was harvested using the miRNeasy purification kit following the manufacturer's protocol. Reverse transcription was performed using a Superscript reverse transcription kit (Invitrogen) with two gene specific primers to COX-1/COX-3 (TCAGAGCTCTGTGGATGGTCG) and GAPDH (AGCCAAATTCGTTG). PCR was performed following the Platinum Pfx protocol given earlier with 20, 40 and 45 cycles for GAPDH, COX-1 and COX-3 respectively using the following PCR primer pairs: GAPDH (GTCGCCAGCCGAGC, ACCTTGCCACAGCCT), COX-1 (CCATGAGCCGGAGTCTCTTG, CTGATGTAGTCATGTGCTGAGTFGTA), COX-3(CATGAGCCGTGAGTGCGA, CTGATGTAGTCATGTGCTGAGTTGTA).

siRNA Knock-Down of COX-3 Expression

siRNAs (100 nanomoles/150,000 cells) were transfected into MEG-01 and K562 cells using lipofectamine RNAiMAX reagent (Invitrogen) using the recommended reverse transfection protocol and incubated at 37° C. for 72 hours. Cells were harvested and half used for RNA purification and RT-PCR analysis and the other half analyzed by immunoblot following the protocols used for the cell line screen.

Three different COX-3 specific siRNAs (targeting intron-1) were used together in this paper. Two of these (GGUGGAGCCUUGAAUGCCA, and CCUGGUGGAGCCUUGAAUG) were designed using the siRNA design tool available from Dharmacon. The third COX-3 siRNA (CUCAUCUCUCUCCUCUGCA) was also prepared by Dharmacon based upon the siRNA used successfully by Nurmi et. al (19). COX-1/COX-3 siRNAs were purchased from Dharmacon (siGenome Smart Pool) and contain a mixture of four different siRNAs all targeting exons 10 and 11 of the COX-1 and COX-3 mRNAs. The sequence of these COX-1/COX-3 siRNAs are GGAAUUGUAUGGAGACAUU, GAACAUJGGACCACCACAUC, CAAGAGGUUUGGCAUGAAA, GGGAAUGGCAGCAGAGUUG. As a negative control, cells were transfected with 100 nanomoles of Dharmacon's non-targeting siRNA #2.
1. Vane, J. R. (1971) Inhibition of prostaglandin synthesis as a mechanism of action for aspirin-like drugs. Nat New Bioi, 231, 232-235.
2. Roth, J., Rummel, C., Barth, S. W., Gerstberger, R. and Hubschle, T. (2006) Molecular aspects of fever and hyperthermia. Neural Clin, 24, 421-439, v.
3. Khan, A. H., Carson, R. J. and Nelson, S. M. (2008) Prostaglandins in labor—a translational approach. Front Biosci, 13, 5794-5809.
4. Moncada, S. and Vane, J. R. (1979) The role of prostacyclin in vascular tissue. Fed Proc, 38, 66-71.
5. Toomey, D. P., Murphy, J. F. and Conlon, K. C. (2009) COX-2, VEGF and tumour angiogenesis. Surgeon, 7, 174-180.
6. Simmons, D. L., Botting, R. M. and Hla, T. (2004) Cyclooxygenase isozymes: the biology of prostaglandin synthesis and inhibition. Pharmacal Rev, 56, 387-437.
7. Xie, W. L., Chipman, J. G., Robertson, D. L., Erikson, R. L. and Simmons, D. L. (1991) Expression of a mitogen-responsive gene encoding prostaglandin synthase is regulated by mRNA splicing. Proc Nat/Acad Sci USA, 88, 2692-2696.
8. Chandrasekharan, N.Y., Dai, H., Roos, K. L., Evanson, N. K., Tomsik, J., Elton, T. S. and Simmons, D. L. (2002) COX-3, a cyclooxygenase-1 variant inhibited by acetaminophen and other analgesic/antipyretic drugs: cloning, structure, and expression. Proc Nat/Acad Sci USA, 99, 13926-13931.
9. Snipes, J. A., Kis, B., Shelness, G. S., Hewett, J. A. and Busija, D. W. (2005) Cloning and characterization of cyclooxygenase-1b (putative cyclooxygenase-3) in rat. j Pharmacal Exp Ther, 313, 668-676.
10. Kis, B., Snipes, J. A., Gaspar, T., Lenzser, G., Tulbert, C. D. and Busija, D. W. (2006) Cloning of cyclooxygenase-1b (putative COX-3) in mouse. Injlamm Res, 55, 274-278.
11. Qin, N., Zhang, S. P., Reitz, T. L., Mei, J. M. and Flores, C. M. (2005) Cloning, expression, and functional characterization of human cyclooxygenase-1 splicing variants: evidence for intron 1 retention.] Pharmacal Exp Ther, 315, 1298-1305.
12. Atkins, J., Gesteland, R F. (2010) Recoding: Expansion of Decoding Rules Enriches Gene Expression. Springer.
13. Dinman, J. D. (2012) Control of gene expression by translational recoding. Adv Protein Chern Struct Bioi, 86, 129-149.
14. Ivanov, I. P. and Matsufuji, S. (2010) In Atkins, J. F. and Gesteland, R. F. (eds.). Springer New York, Vol. 24, pp. 281-300.
15. Michel, A. M., Roy Choudhury, K., Firth, A. E., Ingolia, N. T., Atkins, J. F. and Baranov, P. V. (2012) Observation of dually decoded regions of the human genome using ribosome profiling data. Genome Res.
16. Gerashchenko, M. V., Su, D. and Gladyshev, V. N. (2010) CUG start codon generates thioredoxinfglutathione reductase isoforms in mouse testes.] Bioi Chern, 285, 4595-4602.
17. Kochetov, A. V. (2008) Alternative translation start sites and hidden coding potential of eukaryotic mRNAs. Bioessays, 30, 683-691.
18. Kozak, M. (2002) Pushing the limits of the scanning mechanism for initiation of translation. Gene, 299, 1-34.

### Example 2

### Translational Recoding of Cyclooxygenase-1 and Nucleobindin Genes Produces Proteins that Function in an Evolutionarily-Ancient Autophagic Innate Immunity Pathway

Heme binding peroxidases were among the first enzymes evolved to deactivate reactive oxygen species (ROSs) in earth's early oxygenated environment. Later, unicellular and multicellular organisms utilized these peroxidases to not only deactivate ROSs but enzymatically exploited them in defense against invading pathogens. A major superfamily of heme peroxidases arising from these ancient enzymes is the large peroxidase-cyclooxygenase superfamily, which spans prokaryotic and eukaryotic organisms. Members of this superfamily are heavily involved in innate immunity often by generating products that blunt the effect of invading organisms. In unicellular organisms, fungi, and plants, certain members of this superfamily, which we term cyclooxygenase-like peroxidases (CLPs), contain motifs similar to “modern” mammalian cyclooxygenase-1 and 2 isoenzymes which are targets of aspirin-like drugs. These structural features include evolutionarily conserved alpha helices, peroxidase site, and a reactive tyrosine at a primordial cyclooxygenase active site. However, CLPs are translated in cytosol, lack disulfide bonds, and are un-glycosylated unlike cyclooxygenases.

In lower eukaryotic organisms (e.g. fungi, algae) and in plants these CLPs make oxygenated lipids (lipoxins) that act intra-cellularly in host innate immunity-often to evoke apoptosis in invaded cells.

Mammalian cyclooxygenase (COX) isoenzymes arose recently in evolution and are only found in vertebrates. COX-1 and COX-2 isoenzymes synthesize prostaglandin H2 which downstream synthases metabolize to form many prostanoids, which are typically released from the cell to act in a paracrine manner. Thus, the function of prostanoid synthesis is consistent with the late evolutionary arrival of COX-1 and COX-2 as prostaglandin synthesizing enzymes because prostanoid isomers play complex roles in maintaining tissue and organ homeostasis in advanced multicellular organisms. For example, COX-1 is constitutively expressed in multiple tissues and cells including the gut and platelet where COX-1 maintains prostanoid mediated gut homeostasis and thrombosis, respectively. Likewise, COX-2 is critical to ovulation, inflammation, embryonic implantation, nociception, and fever and is dysregulated in a number of pathological conditions such as inflammatory diseases and cancer.

Over the last two decades, we found that under certain circumstances such as starvation, viral infection, apoptosis, or in the brain of dogs, rodents, and humans; certain splice variants of both COX-1 and COX-2 retain intronic or partial intronic sequences in or near the N-terminal signal peptide. These retained introns or partial introns typically cause frame shifts in the COX mRNA rendering it ineffective with regard to synthesizing COX proteins by the scanning method of translation. These “non-functional” mRNAs are inducible and in certain circumstances are abundant, exceeding the levels of COX-1 and COX-2 mRNAs.

A splice variant of COX-1 that retains intron-1, denoted as COX-3 or COX-1b, cloned from canine brain, is expressed widely in mammals. But retention of intron-1 in rat and human causes a frameshift in the coding region and, as mentioned above, is predicted to only produce a small peptide unrelated to cyclooxygenase by the scanning model. We showed, however, that catalytically active glycosylated COXs are translated from rat COX-3 mRNA through translational recoding mechanisms of ribosomal frameshifting and translation initiation at alternative start codons. We also found that recoding of this transcript produces four other rat COX-3 recoded proteins that are cytosolic (as opposed to being in the lumen of the endoplasmic reticulum [ER] like COX-1 and COX-2), are unglycosylated, and do not catalyze the prostaglandin synthesis. Three of these recoded proteins initiate translation at downstream AUGs that are evolutionarily conserved among mice, human, and rat. These translationally recoded rat COX-3 products are here referred to as rc57, rc50, and rc44 (for their electrophoretic mobility). The fourth protein (r68) is synthesized from a non-AUG translation start site.

The r57, r50, and r44 proteins show marked similarity to evolutionarily more ancient members of the peroxidase-cylooxygenase superfamily. They are cytosolic, contain heme binding and peroxidase sites, and a reactive tyrosyl-residue at a cyclooxygenase-like site. Because of this similarity to CLPs in unicellular organisms we reasoned that these might function in innate immunity at the cellular (rather than tissue or organ) level. This postulate was buttressed by a previous finding by our laboratory that non-glycosylated COX proteins could interact with nucleobindin (Nuc), a multifunctional protein implicated in innate immunity that serves an intracellular role in signaling.

Among its many putative functions, Nuc inhibits Galphai3, regulates apoptosis, and induces lupus in rats. Nuc contains a cleavable N-terminal signal peptide, two ErF-hands, DNA binding domain, and Galphi3 binding domain. However, despite the fact that its N-terminal signal peptide should direct it to membrane structures, Nuc localizes to both luminal and cytosolic surfaces of golgi, cytosol, and extracellular matrix. The mechanism by which Nuc, containing a signal peptide, localizes to cytosol has been to this point a mystery.

Here we show that cytosolic Nuc (cNuc) occurs through translational recoding similar to that which produces rc57, rc50, and rc44, and that this process directs cNuc to cytosol. Through localization to the cytosolic compartment, translationally recoded rc57, rc50, rc44, and cNuc physically/functionally interact to initiate cellular processes—specifically autophagy. We determined rc57, rc50, and rc44 are members or the critical ATG9 complex that governs formation of autophagosomes. Moreover, these proteins act synergistically with Nuc to form large autophagosome structures, termed mega-autophagosomes, implicated in virus replication. Reactive tyrosyl residue as well as heme binding are essential to this function and demonstrate yet undefined enzymatic roles of rc57, rc50, and rc44 in autophagosome trafficking and mega-autophagosome formation. Furthermore, recoded cNuc in rats regulate autophagic flux, blocking it before amphisome formation. Together these data reveal a new role of COX genes in intracellular innate immunity through an autophagic pathway, which we propose represents their ancient function.

### Results

Evolutionary Comparison of r57, r50, and r44 with CLPs by I-TASSER

Next generation sequencing techniques have identified genomes of 100s of micro-organisms some of which have now revealed CLPs. These discoveries suggest an evolutionary ancient role in innate immunity for CLPs in unicellular life. The secondary structure prediction program I-TASSER generates 3D structures based upon threading/fold recognition methodology. We used this program to predict structures for rc57, rc50, and rc44. These predicted structures were then compared to known or predicted PDB structures of non-animal CLP enzymes.

All 10 templates used were of COX-1 or COX-2 PDB files due to sequence homology. Next, I-TASSER also identified proteins as having highly similar structures to rcCOXs. Predictably, rcCOXs are shown to be similar to members of the peroxidase-cyclooxygenase superfamily such as mammalian COX, α-dioxygenase, lactoperoxidase, and myeloperoxidase. C-scores for the rc57, rc50, and rc44 models are 1.85, 1.63, and 1.61 respectively while TM-scores are 0.98+/−0.05, 0.94+/−0.05, and 0.94+/−0.05 respectively. The RMSD scores for the predicted models of the rc57, rc50, and rc44 are 3.4+/−2.4 Å, 3.7+/−2.5 Å, and 3.4+1-2.4 Å respectively.

Similar to most non-animal CLPs, rCOXs do not contain membrane binding or dimerization domains. However they do contain the C-terminal Nuc binding domain found in COX-1 and COX-2. The predicted structures for both the r57 and r50 have a globular peroxidase site formed by alpha helices (numbering from N-terminus) four, twelve, and seventeen and loop between the fourth and fifth alpha helix. The peroxidase site for r44 is not well structured due to missing alpha helix 4. Known heme binding residues His207 and His388 (numbering based upon ovine COX-1) are situated within the cleft described above in a way that would coordinate with heme for r57 and r50. In contrast, r44 lacks His207. Each rCOX contains a predicted active tyrosyl residue (Tyr385) situated on the opposite side of the heme to the peroxidase site (similarly found in COX-1 and COX-2) which would be indicative of an ability to oxygenate lipids. Unlike r50 and r44, r57 contains the Arg120 residue important for coordination with the carboxyl group of fatty acids which directs the hydrophobic chain into the oxygenase site. All rCOXs contain Tyr355 residue which is also important for interacting with the carboxyl group on fatty acids. Together these data suggest that rCOXs have peroxidase activity and potential lipoxin generating activity, localize to cytosol, do not homodimerize or bind membrane directly Also, there is potential for synthesis of differing products due to retention of Arg120 for r57 but absent for r50 and r44 lacking alpha helix 4.

Comparison of Bacterial CLPs with rCOXs.

CLPs from unicellular organisms have been recently identified in distantly related bacterial species. Nitrosomas europaea, a gram negative proteobacterium, and Mycobacterium vanbaalenii each have CLPs for which we generated predicted structures. Clustal2.1 showed 34%, 35%, and 37% residue homology between CLP in Nitrosomas europea (CLPne) and r57, r50, or r44 respectively. We used 1-TASSER to predict the secondary structure of CLPne to compare the secondary structure of r57, r50 and r44 predicted models. In the top 10 templates used by I-TASSER for modeling CLPne, the top nine are mammalian COXs and the tenth template is oryza sativa fatty acid a-dioxygenase. The CLPne model has a C-score of 0.9, a TM-score of 0.84+/−0.08, and a RMSD score of 5.5+/−3.5 A.

CLPne contains 26 alpha-helices. A cleft is formed by alpha helices (numbering from N terminus) 6, 13, and 17 and loops between alpha helices 5 and 6; 6 and 7; and between 13 and 14. Situated within the cleft are a conserved active tyroslyl-residue important for lipid oxygenase activity and the proximal histidine residue that is important for coordinating with heme. Interestingly, alpha helices 1 and 2 may form a mammalian CLP-like membrane binding domain similar to COX-1 and COX-2, In this case the enzyme would be anchored presumably to the inner surface of the bacterial plasma membrane.

We used Chimera 1.8.1 to visualize secondary structure overlap between CLPne and rCOXs. CLPne secondary structure showed high overlap with both r57 and r50. Interestingly, both the Tyr385 and His388 of these rCOXs overlap with the proposed proximal His ligand to heme and active tyrosyl residue of CLPne in the cleft described above, CLPne lacks the His207 of the rCOXs which is instead an Asp residue.

Next, Mycobacterium vanbaalenii CLP (CLPmv) also contains 26 alpha helices similar to CLPne. Like CLPne and rcCOXs, CLPmv structure is globular with a cleft formed where a proximal His could potentially coordinate with a heme group and a Tyr residue located adjacent to the heme group important for oxygenase activity. The distal His ligand residue is absent and is instead an Asp like CLPne. Likewise, CLPmv secondary structure also has high overlap with rcCOXs wherein the alpha helices, proximal His ligand, and Tyr385 are situated at the peroxidase site. Of nine different enzymes of the large peroxidase-cyclooxygenase superfamily, all 9 enzymes have conserved the active tyrosyl residue and 8 have conserved the proximal His ligand (the exception being lactoperoxidase). Just COX-1 and rcCOXs contain the distal His ligand. Focusing on the secondary structure we found strong alpha helical homology in the catalytic domain of all 9 enzymes but very little overlap at the N-termini. These data indicate the strong similarity between ancient bacterial CLPs and rcCOX proteins. In summary, predicted structures for rcCOXs and bacterial species show a high degree of similarity. These similarities are also seen in other members of the peroxidase-cyclooxygenase superfamily, in particular, each shows conserved helices, heme-binding, and a potential reactive tyrosyl for lipid oxidation.
Confocal Microscopy of rCOXs Identifies a Role in Autophagy

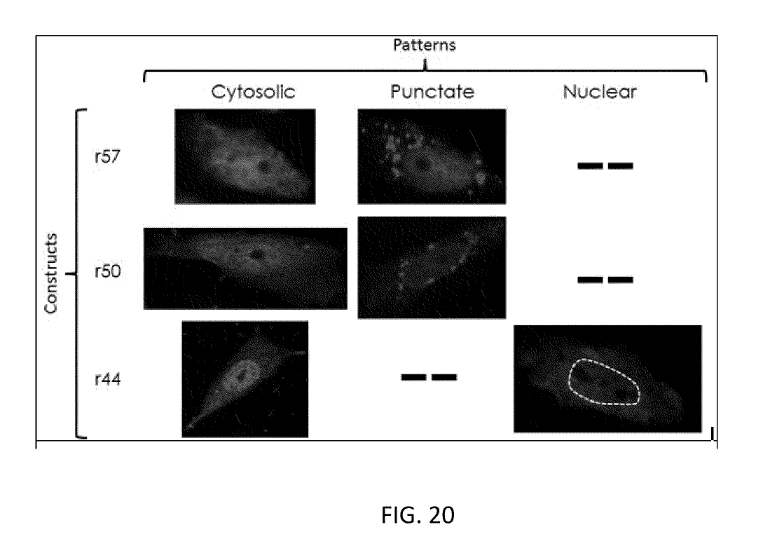

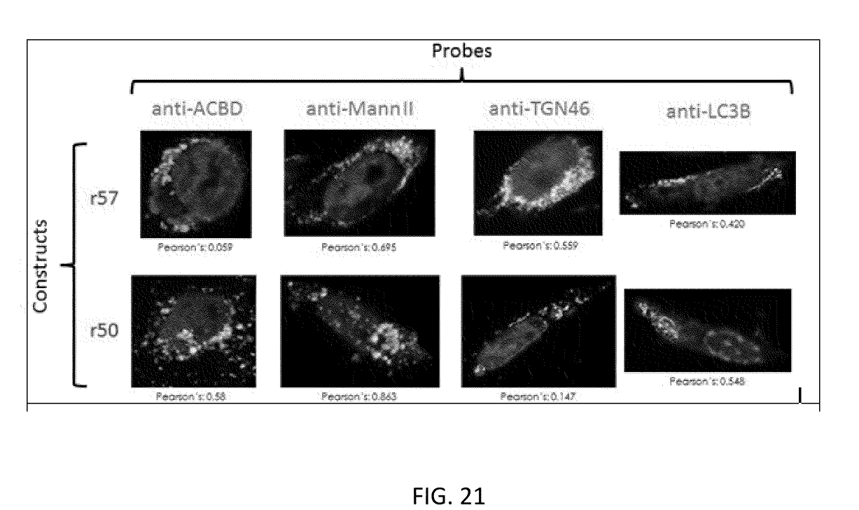

We hypothesized that the rCOXs would be cytosolic due to their lack of a signal peptide as well as their I-TASSER-predicted structures, therefore, we used confocal microscopy to image CHO cells transiently transfected with FLAG-tagged receded COXs to test this hypothesis. Confocal micrographs showed cells transfected with r57 or r50 to exhibit either cytosolic or punctate patterns, in any given experiment, some transfected cells exhibited primarily cytosolic distribution while others exhibited punctate localization, indicative of localizing to organelle-like structures. These putative organelles were typically seen near or around the nucleus suggesting a golgi pattern (FIG. 20). We tested for golgi localization by dual-labeling cells transfected with FLAG-tagged r57 or r50 with probes against the rCOX proteins in combination with markers targeting either cis-golgi (ACBD), intermediate golgi (mannosidasc II), or trans-golgi (TGN46) apparati. Very little r57 co-localized with cis-golgi marker (Pearson's coefficient: 0.059); however, r57 co-localized strongly with intermediate and trans-golgi markers (Pearson's coefficient: 0.695 and 0.559 respectively FIG. 21). In contrast, r50 co-localized with cis and intermediate golgi markers (Pearson's coefficient: 0.58 and 0.863 respectively) more than trans-golgi (Pearson's coefficient: 0.127).

In the process of these analyses, it was noted that not all of the punctate structures observed for r57 and r50 could be accounted for by golgi staining. By analyzing a variety of markers for intracellular membrane structures we identified these organelles as LC3B containing autophagosomes. Thus, when r57 and r50 exhibit punctate pattern they are partitioning between golgi bodies and autophagosomes.

Unlike r57 and r50, r44 does not exhibit a punctate pattern, does not localize to golgi or autophagosomes and instead exhibits cytosolic and intranuclear localizations. As with r57 and r50, which were predominantly either cytosolic or membrane associated, depending on the state of the cell, r44 was either predominantly cytosolic or nuclear in any given cell.

r57 and r50 Co-Localize with ATG9-RFP while r44 Co-Localizes with ATG9-RFP when in Cytosol but not within the Nucleus

Currently, 31 autophagy related (ATG) genes have been identified and of these, ATG9 is associated with innate-immunity autophagy. Additionally, ATG9 is a multi-transmembrane chaperone known to be involved in the transport of membrane from golgi to autophagosomes. The localization pattern of ATC9 has been described to be punctate and cytosolic depending on the state of the cell. ATG9 has been shown to reside on the golgi, endosomes, and autophagosomes. Due to the similar localization pattern seen for rc57 and rc50, we hypothesized the rc57 and rc50 traffic between golgi and autophagosome with ATG9.

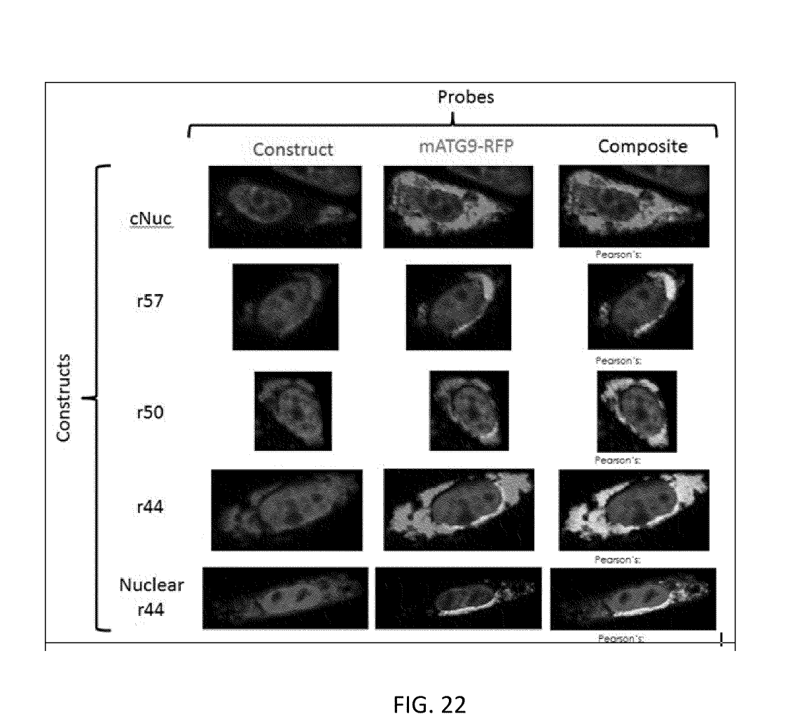

To test this hypothesis, we co-transfected cNuc, rc57, rc50, or rc44 with C-terminal RFP labeled—ATG9 and monitored co-localization by confocal microscopy. Near complete overlap of expression between rcCOXs and ATG9 was observed with Pearson coefficients equal to 0.973, 0.954, and 0.928 for rc57, rc50, and rc44 respectively. ATG9-RFP co-localized with rc57 and rc50 at two sites: either near the nucleus indicating accumulation at the golgi or throughout the periphery of the cell. ATG9-RFP only co-localized with rc44 in the periphery of the cell when rc44 was cytosolic and not when rc44 was intranuclear (FIG. 22). To assure that this co-localization was not an artifact of overexpression of tagged proteins we assessed the localization of rc57, rc50, and rc44 with endogenous ATG9 using an anti-ATG9 antibody probe. Again, near identical overlap between rcCOXs and ATG9 was observed (Pearsons coefficient: between 0.7 and 0.8). However, when rc44 was in the nucleus endogenous ATG9 was found in a punctate pattern in the periphery of the cell.

Because confocal microscopy could not distinguish between colocalization and actual interaction between rcCOXs and ATG9 we performed co-immunoprecipitation experiments by co-transfecting rcCOXs with hemaglutanin (HA) tagged ATG9 and immunoprecipitating using anti-HA antibody to isolate the ATG9 complex. In rc57 transfected cells, rc57 was co-immunoprecipitated with ATG9 demonstrating these proteins, if not binding partners, are in the same protein complex.

We observed cNuc was found in a punctate pattern at the periphery of cells mutually excluded from ATG9-RFP while ATG9-RFP was found in a golgi-like pattern around the nucleus.

### Site-Directed Mutagenesis Gives Evidence for Recoding Mechanisms of Nucleobindin

We previously identified nucleobindin as a binding partner of non-glycosylated COX proteins. The rc57, rc50, and rc44 all contain the nucleobindin interaction domain and are non-glycosylated and, therefore, represent potential binding partners for nucleobindin, but only for a cytosolic form of this protein. This interaction is functionally important because nucleobindin is implicated in autophagy/innate immunity.

Previous studies have shown there are two pools of Nuc, one found in the lumens of ER/golgi/endosome organelles and a cytosolic form. Since Nuc contains a signal peptide, only the organelle pool should exist. Currently, a mechanism for how Nuc is translated into cytosol has not been defined. Because recoding results in synthesis of cytosolic proteins from signal peptide containing COX transcripts, we tested whether a similar phenomenon occurs with Nuc.

A clue to the fact that such recoding occurs in vivo is indicated by studies that show the cytosolic Nuc and organelle bound Nuc to be relatively the same size. This indicated that a translation initiation site for the creation of cNuc through recoding potentially is located near the cleavage site for the signal peptide resulting in a cytosolic protein that would be the same size as proteolytically processed luminal Nuc. We therefore looked for potential start codons shortly downstream of the initiating AUG. For mouse and rat Nuc but not human, a conserved downstream AUG is found 6 codons from the signal peptide cleavage site. These were mutagenized in Myc-tagged or non Myc-tagged constructs, transfected into CHO cells, and analyzed via probing western blots with appropriate antibodies. This experimentation determined that initiation at these internal AUGs produce a cytosolic Nuc of the correct size seen in in vivo studies.

We then tested whether translation initiation at the internal AUG resulted in similar localization patterns as reported. We transfected non-mutagenized Nuc and M1KNuc into CHO cells and analyzed the localization using confocal microscopy. We found Nuc in a punctate pattern as either near the nucleus or in the periphery while M1KNuc localized throughout the cytosol and at times formed a large ring like structure. These results implicates Nuc localization being dependent upon recoding mechanism via the use of downstream initiation start AUG similar to rcCOXs.

Nuc Localizes to Membrane Structures Whereas cNuc Localizes Mainly to Cytosol but Also Mega-Autophagosomes

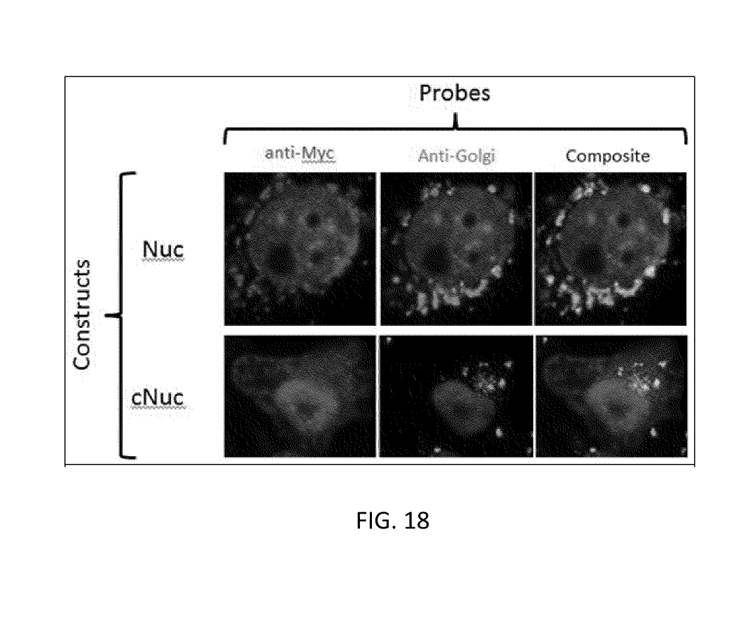

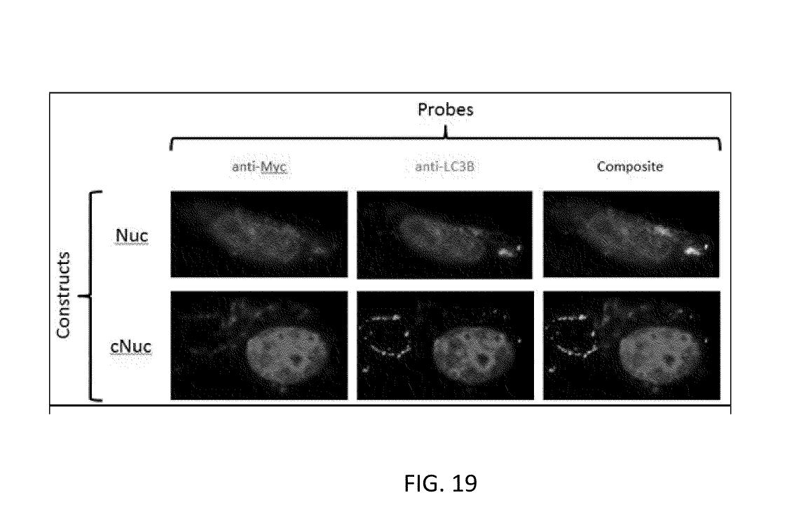

In agreement with previous studies, we obtained confocal microscopy images that showed Myc-tagged cNuc was largely cytosolic and that Myc-tagged Nuc exhibited the same punctate pattern (FIG. 18). Nuc localized to golgi bodies (ACBD marker) as reported before but we also observed Nuc localized to the autophagosome marker LC3B marker. We also observed that cNuc associated around large ring-like structures (FIG. 19). LC3B also co-localized with cNuc around these large structures that have been previously been identified in rat pancreatic cells infected with coxsackievirus to be mega-autophagosomes. However, we never observed mega-autophagosomes in Nuc transfected cells.

Recoded COXs Translocate with cNuc Around Mega-Autophagosomes and Act Synergistically in Both the Formation of Mega-Autophagosomes and Localization of cNuc to LC3B

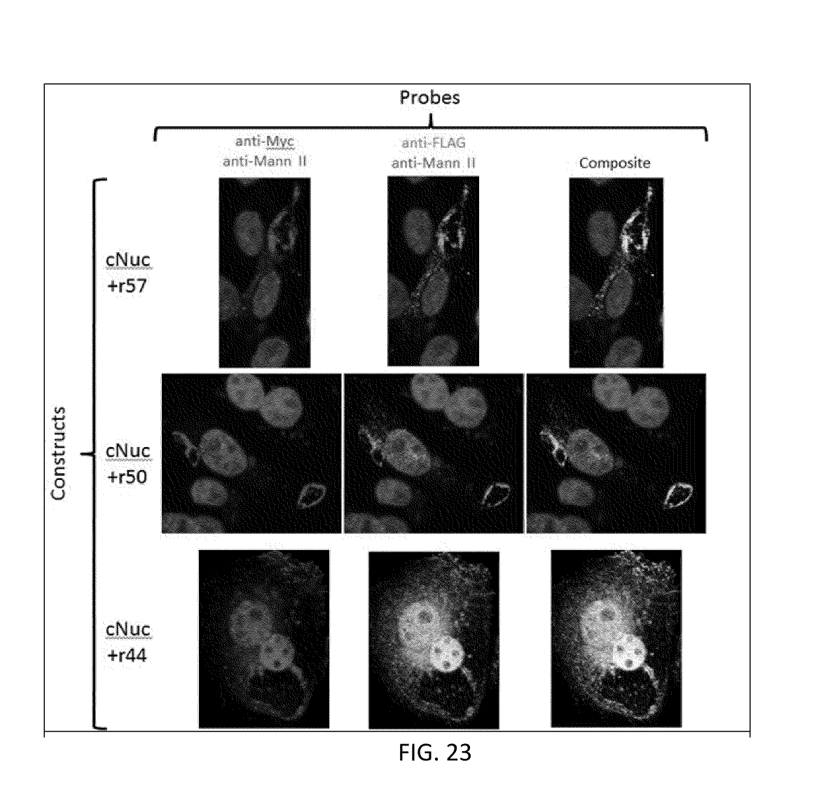

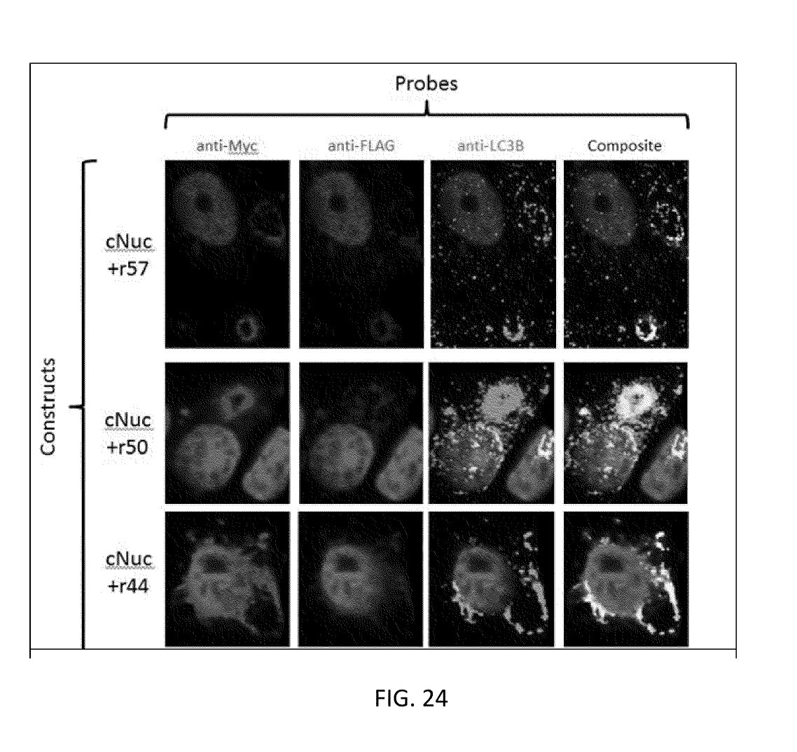

Previously, we showed COX and Nuc interaction was dependent on COX being unglycosylated or hypo-glycosylated. We hypothesized that r57, r50, and r44, being unglycosylated and cytosolic, would co-localize and/or interact with cNuc. When we co-expressed Myc tagged cNuc with FLAG tagged rCOXs, r57 and r50 both translocated from golgi to co-localize with cNuc around mega-autophagosomes (FIG. 23). Likewise, r44 co-localized with cNuc around mega-autohphagosomes (FIG. 24). Mega-autophagosomes were usually found near the plasma membrane when cNuc was co-expressed with r57 or r50 while r44/cNuc formed mega-autophagosomes were situated near the nucleus. Mega-autophagosomes never formed in non-transfected cells, suggesting that the action of either rCOXs or cNuc is intracellular and not paracrine.

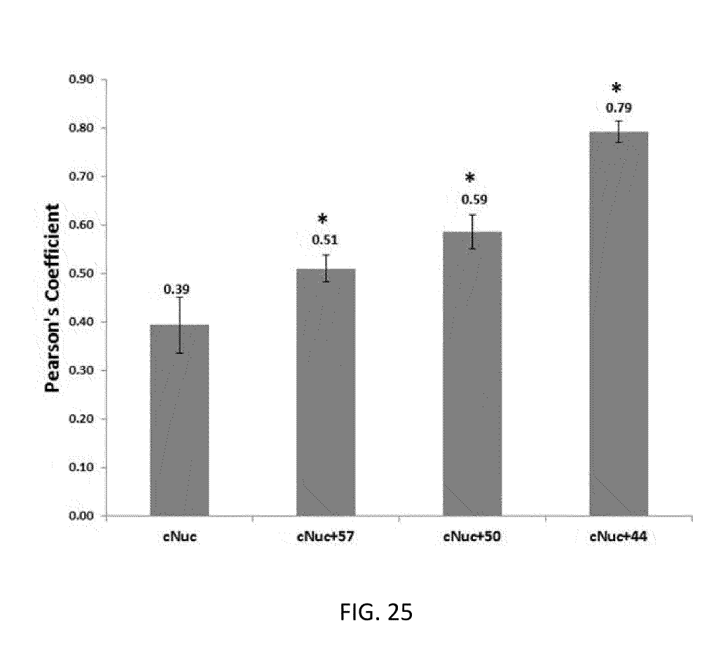

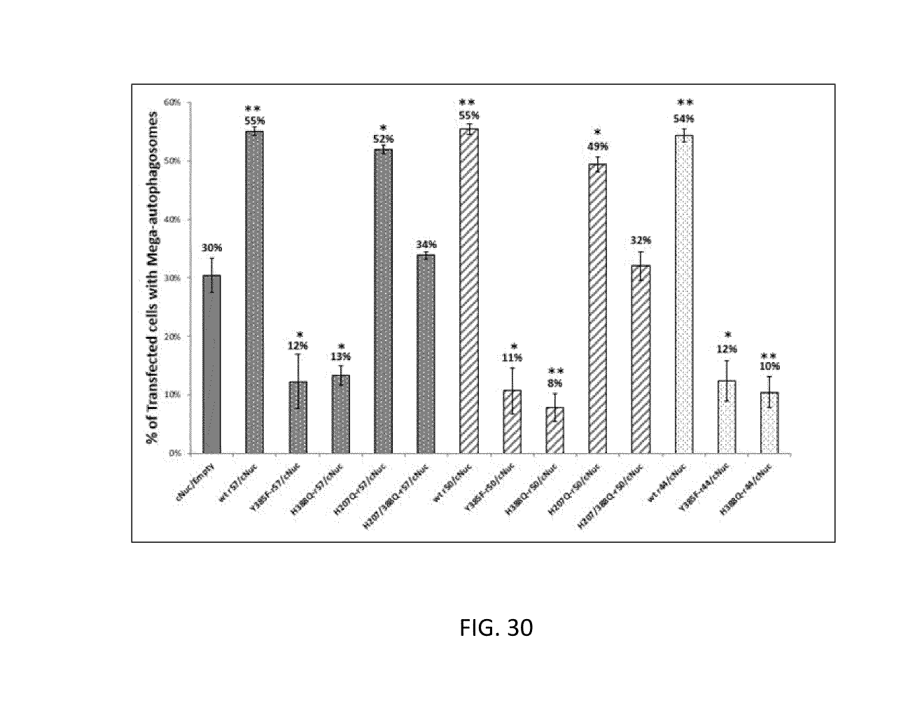

Interestingly, we found a statistically significant increase in co-localization of cNuc to LC3B when co-expressed with r57, r50, or r44 (p-value=0.04, 0.005, and 7.4×10-5 respectively) than compared to cNuc expressed alone (FIG. 25). Also, co-transfection doubled the number of transiently transfected CHO cells with mega-autophagosomes compared to cNuc alone (FIG. 30). These data suggest rCOX proteins are important for the formation of mega-autophagosomes.

Site-Directed Mutagenesis of Important Catalytic COX Residues Influence Mega-Autophagosome Formation

In FIG. 30 we find cNuc alone lead to mega-autophagosome formation in 30% of transfected cells while co-transfection with rCOXs doubles the percentage of cells containing mega-autophagosomes. To test whether rCOXs effect on mega-autophagosome formation is due to peroxidase activity, we mutated key residues important for coordinating with heme. Heme is an important co-factor required for peroxidase and cyclooxygenase activity. The two most important residues for coordinating heme are the proximal histidine ligand (His388) and the distal histidine ligand (His207). Since r44 does not contain a distal histidine, only it's proximal histidine ligand was mutated. These constructs were then co-transfected with cNuc and visualized using confocal microscopy with triple-labeling against Myc-tagged cNuc, FLAG-tagged rCOXs, and LC3B. We then counted the number of transfected cells that formed a mega-autophagosome when these rCOX constructs were co-transfected with cNuc. The distal ligand mutated contructs (H207Q-r57 and H207Q-r50) co-transfected with cNuc did not effect the number of transfected cells with mega-autophagosome compared to wild-type rCOXs co-transfected with cNuc. While co-transfecting H388Q-r57, H388Q-r50, or H388Q-r44 with cNuc caused the number of transfected cells with mega-autophagosomes to be half that of cNuc alone transfected cells indicative of a dominant negative effect. Mutating the proximal and distal histidine ligands (H388/207Q-r57 and H388/207Q-r50) blocked wild type rCOX mega-autophagosome induction and just around 30% of the cells contained mega-autophagosomes.

We then tested whether the effects described above were due to lack of heme (which results in loss of structure) or due to catalytic activity. We mutated the active tyrosyl (Tyr385) to a Phe. We observed similar dominant negative effects with Tyr385 (Y385F-r57, Y385F-r50, and Y385F-r44) mutated constructs. These results suggest the rCOXs possess catalytic activity and that the bioactive molecule acts in an intracellular manner in the formation of mega-autophagosomes.

Mutation of Active Tyrosine (Tyr385) and Both Proximal (His388) and Distal (His207) Histidine Ligands Disrupts cNuc/rCOX Co-Localization but not Mutation of Either His388 or His207

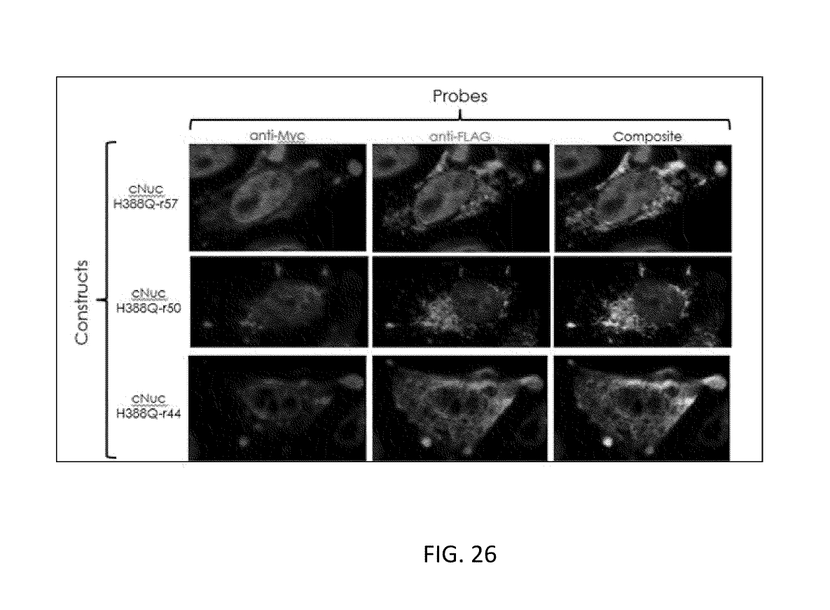

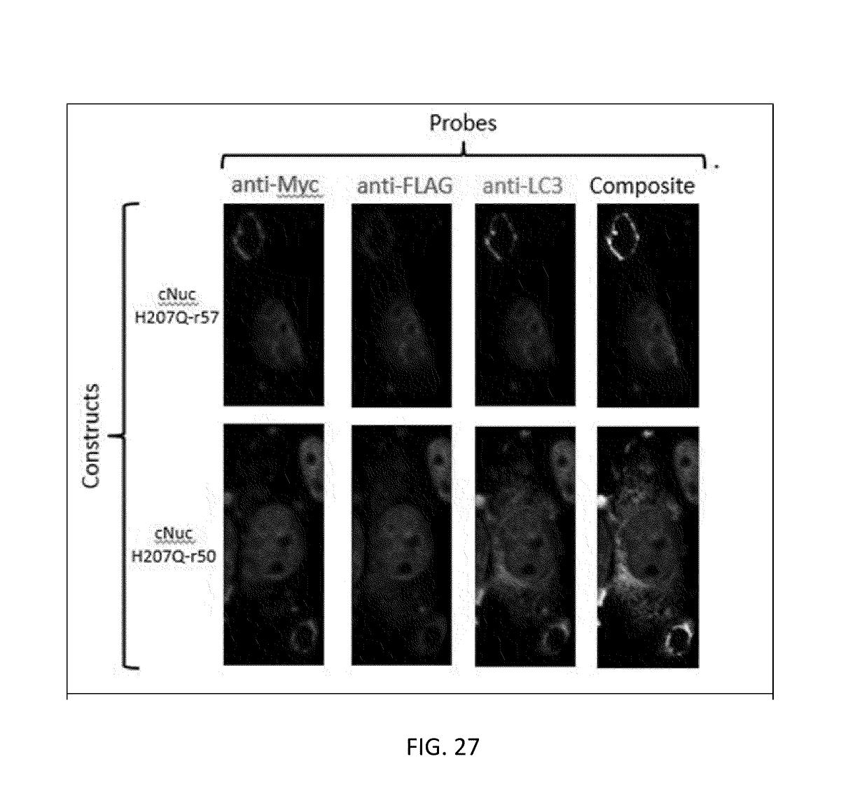

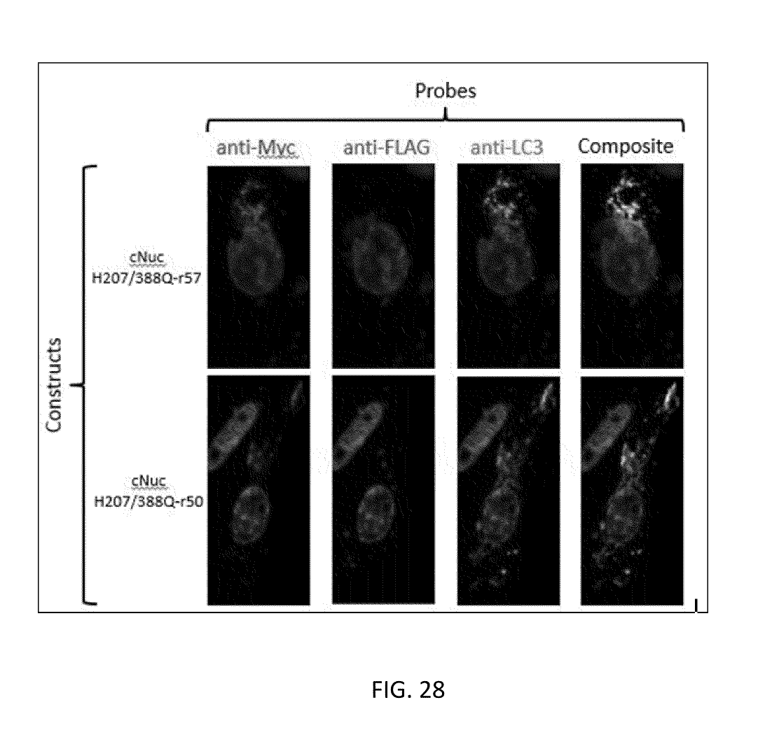

We then looked at the localization pattern of the mutated constructs described above for disruption of cNuc and rCOX localization. The H388Q-r57, H388Q-r50, and H388Q-r44 constructs continued to co-localize with cNuc but mostly at punctate autophagosomes located in the periphery of the cell (FIG. 26), We observed the H207Q-r57 and H207Q-r50 constructs co-localized with cNuc around mega-autophagosomes similar to wild type rCOX/cNuc transiently transfected cells (FIG. 27). The constructs H388/207Q-r57 and H388/207Q-r50 blocked rCOX co-localization with cNuc and was instead localized in large patches around the nucleus, cNuc was found at the periphery of the cell with autophagosomes (FIG. 28).

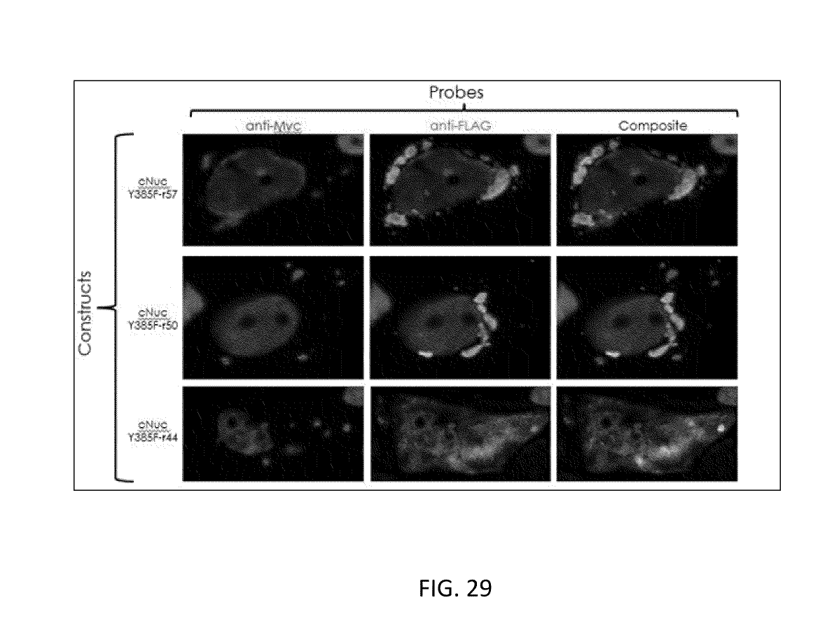

Confocal microscopy images of Y385F-r57 or Y385F-r50 co-transfected with cNuc showed no co-localization with cNuc. Instead we saw Y385F-r57 and Y385F-r50 in enlarged globular structures situated around the nucleus, similar to H388/207Q-r57 and H-1388/207Q-r50 constructs co-transfected with cNuc. cNuc was also found in punctate structures in the periphery of the cell localized to autophagosomes. Images demonstrated that Y385F-r44Y remained cytosolic and co-localized with cNuc at punctate structures in the periphery of the cell. There were some instances Y385F-r44 was in the nucleus suggesting that cyclooxygenase activity is not required for r44 localization into the nucleus (FIG. 29).

cNuc Blocks Autophagic Flux Before Amphisome Formation

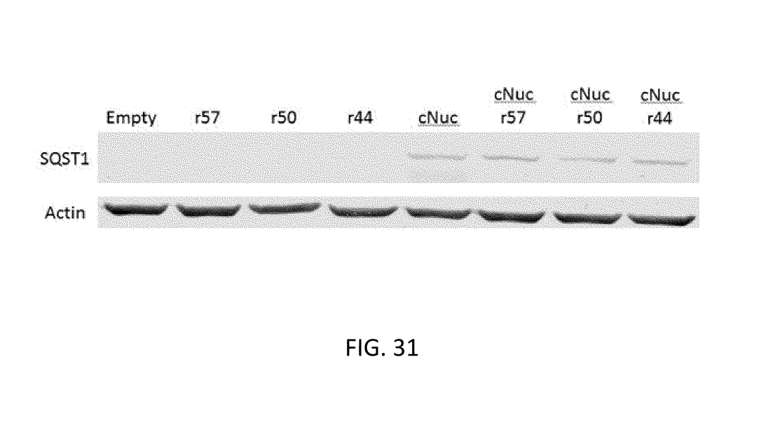

We investigated autophagic flux disruption due to mega-autophagosomes reported to being involved with blocking autophagic flux. Sequestrosome 1 (p62) is an ubiquitin-binding scaffold protein that directs ubiquitinated proteins for degradation through binding LC3B in autophagosomes. Accumulation of p62 shows autophagosome flux disruption. Twenty-four hours after transfection, p62 accumulation was found in CHO cells overexpressing cNuc and cNuc with r57, r50, or r44 (FIG. 31) but p62 was not detected in r57; r50, or r44 overexpressing cells.

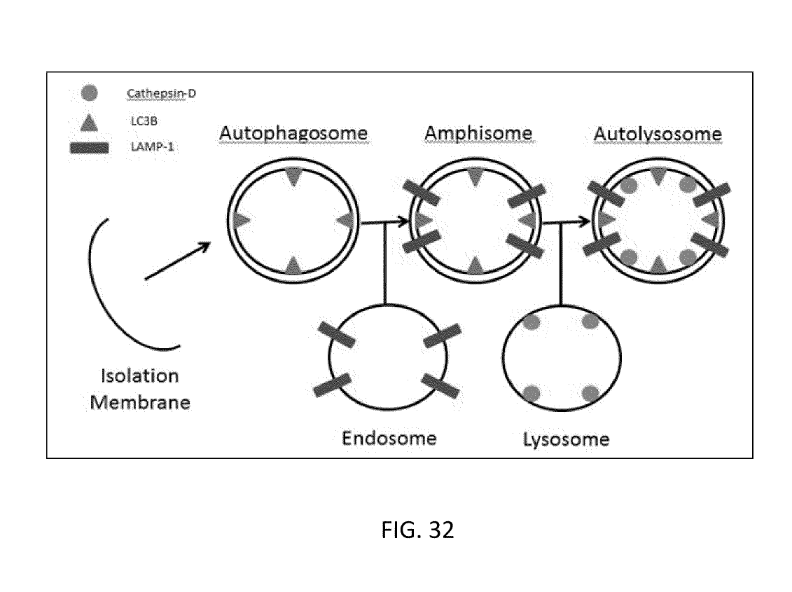

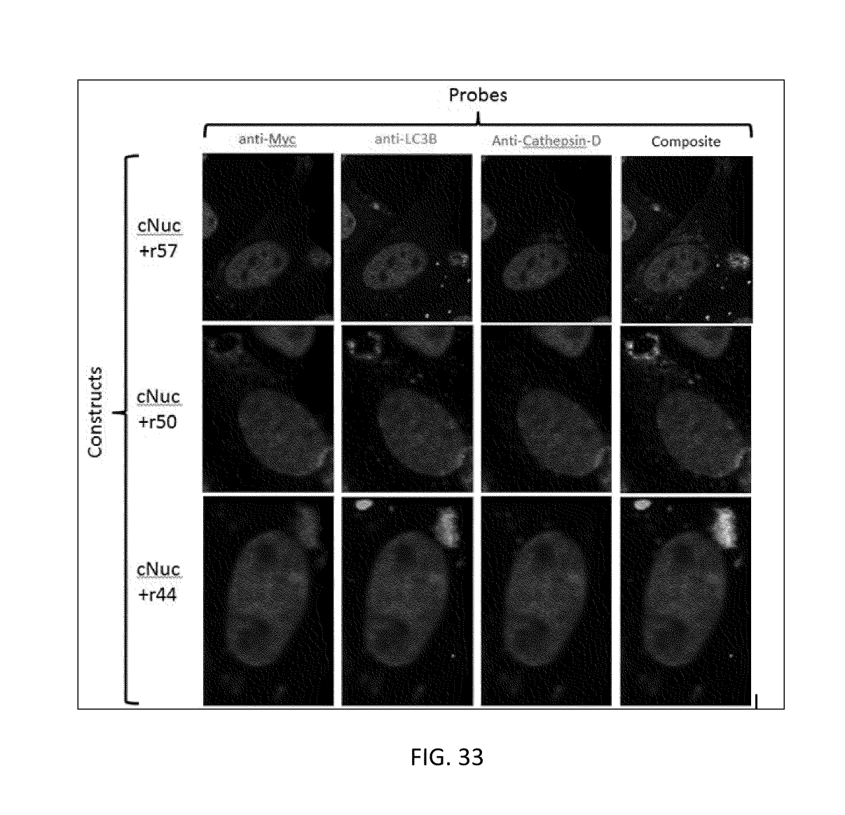

Next, we determined at what point autophagy is blocked. FIG. 32 illustrates the sequence of autophagy maturation and cellular self-digestion. To test for autolysosome formation, mega-autophagsomes formed by the co-transfection of cNuc alone or with rCOXs were probed with the lysosomal marker cathepsin-D. There were no detectable amounts of cathepsin-D found localized to mega-autophagosomes formed by transient co-transfection of cNuc and r57, r50, or r44 (FIG. 33).

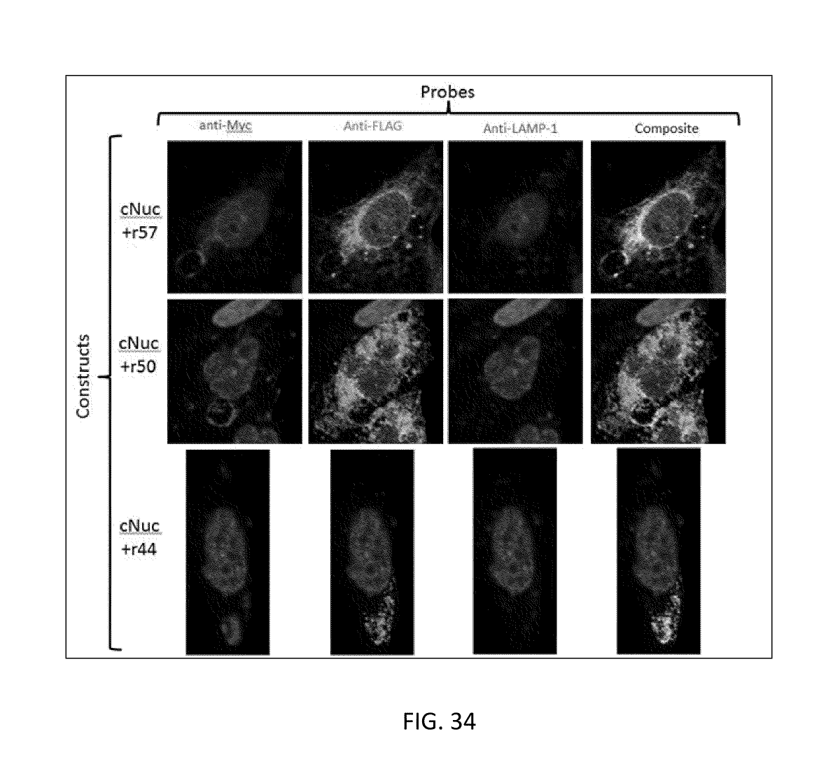

LAMP-1, an amphisome marker, also did not co-localize with mega-autophagosomes formed by cNuc or the co-transfection of cNuc with r57, r50, or r44 (FIG. 34). These results indicate autophagy is blocked before amphisome formation by cNuc and cNuc with r57, r50, or r44.

r50, r44, cNuc Alone and cNuc Co-Transfected with r57 Induce Encephalomyocarditis Viral (EMCV) Replication while r57 Inhibits EMCV Replication

The rCOX constructs being related to ancient unicellular COX and evidence showing their involvement in the formation of innate immune responsive autophagic structures. EMCV is a positive strand RNA picornavirus implicated in human febrile illness. Also, the mTOR small molecule inhibitor rapamycin is shown to stimulate protein synthesis of EMCV proteins (Ref) and that autophagy specifically is shown to promote EMCV viral replication. We infected rCOX or/and cNuc transfected/co-transfected CHO cells with EMCV to determine their influence in viral replication. The media was collected and plaque forming assays were then performed and plaques were counted compared to Empty vector plaque counts.

We found a statistically significant increase in EMCV replication in cells that had been transfected with r50, r44, cNuc, and cNuc co-transfected with r57. There was between 30% to 55% increase in EMCV replication in r50, cNuc, and cNuc with r57 transfected cells while r44 doubled the number of EMCV virions compared to Empty vector. The r57 statistically inhibited virion production by 40% compared to Empty vector. These results indicate the r57, r50, r44, and cNuc play unique roles in innate immunity due to their different actions with EMCV infection.

### DISCUSSION

Adaptation to environmental changes at both the organismal, cellular, and molecular level is important for survival. One mechanism of adaptation is the utilization of early evolved genes for other biological processes. Peroxidases' early role in relieving cellular stress from earth's early oxygenated atmosphere is still evident in nitrosomas europaea's response to the cellular stress of arsenic poisoning by inducing CLPne. Then as a means of fending off invading pathogens, higher organisms such as plants would re-use these putative cyclooxygenases in innate immunity as shown by the induction of tobacco CLP also known as pathogen-induced oxygenase (PIOX) which induces apoptosis. In each of these instances, the action of CLP is in an intracellular manner. Not until animal peroxidases/cyclooxygenases (such as COX-1 or COX-2) evolved are lipoxins seen acting on a tissue/organ level.

Our laboratory has shown that animal genes utilize ancient methods of translation (recoding) to produce enzymes where we show here fulfill a form of innate immunity that evolved in early eukaryotic organisms (autophagy). Recent research has shown time and again a connection between autophagy and immunity and this phenomenon has been referred to as “xenophagy”. In some instances, autophagy is shown to protect host cells from invasion while other pathogens either evade autophagy or utilize autophagy for replication. The multiplicity of outcomes to host invasion by autophagy is due to the complexity in regulation and formation of autophagosomes.

A major contributor to innate immunity is COX. COX to date is known to be the first and rate limiting step in prostanoid production that is involved in organ or tissue based innate immunity such as inflammation. Here we show a new role of COX gene products in innate immunity where they act in an intracellular manner.

Our laboratory has shown rCOXs have tissue specific expression, similarly we show here rCOXs localize to different compartments of CHO cells. Interestingly, organelle localization appears to be dependent upon the preservation of alpha helix 4, r57 and r50 are seen localized to golgi and autophagosomes while r44 is missing alpha helix 4 and is seen in either nucleus or cytosol. Only with the co-transfection of cNuc do we find r44 localized to an organelle structure. Due to lacking the membrane binding domain, being translated into the cytosol because of the lack of signal peptide sequence, and r57, r50, and r44 each found in cytosol by confocal microscopy we speculated they form a complex with a membrane bound protein. One potential membrane bound protein the rCOXs could form a complex with is the golgi/autophagosome localized, membrane transferring chaperone, ATG9.

Recently, p38-IP, an important signaling protein that actives the p38a pathway, was shown to interact with the cytosolic C-terminal domain of ATG9. This result indicates that autophagy is activated via the MAPK pathway, specifically p38a, which is shown to be activated during osmotic stress, similar to COX-3 induction. We found r57, r50, and r44 localize strongly with both exogenous and endogenous ATG9 in a golgi-like or cytosolic pattern. Intranuclear r44 has decrease overlap with exogenous ATG9 but does not co-localize with endogenous ATG9, interestingly, endogenous ATG9 appears punctate indicative of autophagy activation when r44 is found exclusively in the nucleus. We also report r57 as a binding partner of the ATG9 complex through immunoprecipitation experiments.

Our laboratory has found the immunity-associated protein Nuc to be a binding partner of COX and here we show the recoded form of Nuc, cNuc, not only associates with rCOXs but associates with them around virally-associated structures, mega-autophagosomes. Mega-autophagosome is a recently described phenomenon found to occur during coxsackie viral infection in rat pancreatic acinar cells. Mega-autophagosome formation is believed to be caused by the disruption of autophagy by the virus but the process by which this is done has yet to be defined. Also, whether mammalian cells utilize this phenomenon for other biological processes has yet to be determined.

The rcCOXs were shown to accentuate mega-autophagosome formation and we have evidence through point mutations of residues important for oxygenation of lipids that this effect is due to catalytic activity. Mutation of either the proximal His ligand or active tyrosyl residue resulted in a dominant negative effect in mega-autophagosome formation while mutation of the distal histidine did not impede rcCOX/cNuc mega-autophagosome formation. This result indicates that the proximal His ligand and the active tyrosyl residues are highly important for formation of rcCOX/cNuc induced mega-autophagosomes while the distal His plays a minor role. This idea is supported by evolutionary conservation of the proximal His ligand and active tyrosyl residues in both prokaryotic and eukaryotic organisms while the distal His ligand residue is not found in the prokaryotic organisms listed. These results indicate rcCOXs' peroxidase site is more similar to CLP peroxidase sites than to mammalian COX-1/2 peroxidase sites. Interestingly, mutation of both the proximal and distal His ligand residues did not have a dominant negative effect but we instead saw mega-autophagosome formation restored to cNuc levels indicating that the distal His ligand plays a role in heme binding.

Mutation of catalytic residues also effected rcCOX localization with cNuc. Mutation of both the proximal and distal His ligand residues or mutation of active tyrosyl residue resulted in cNuc being mutually excluded from the rcCOXs. While, rcCOXs continued to localize with cNuc when either proximal or distal His ligands were mutated to glutamine. These results indicate that oxygenation of lipids plays a major role in rcCOX localization with cNuc. This idea is supported by crystal structure analysis of homodimerized mammalian COX where oxygenation of the substrate, arachidonic acid, induces structural changes to COX necessary for further activity.

Mega-autophagosome has been shown to correlate with autophagic flux disruption. We also find the same phenomenon here when cells are transfected with cNuc, however, we see that mega-autophagomes lack both the autolysosome marker cathepsin-D and amphisome marker LAMP-1 which indicates that autophagic flux is blocked before amphisome formation. Our results indicate cNuc blocks autophagy and the mechanism needs further investigation. Recently, Jaenisch's group showed defective amphisome formation in Niemann-Pick type C1 (NPC1) disease is due to mutations of NPC1 gene that impedes SNARE complex formation, important for autophagosome/amphisome fusion. One possibility is that cNuc interacts with SNARE proteins directly and impedes their action in organelle fusion. Another is that cNuc has trypsin-like protease activity and could proteolyse either SNARE or NPC1 proteins that are important for autophagy.

EMCV infection also indicates each rCOX play different roles in autophagy due to differences in viral replication rates. Due to evidence of catalytic capabilities of the rCOXs we speculate the differences in EMCV maturation rates is due to differences in rCOX catalytic products. Arg120 has been shown to be a critical residue in substrate orientation. The only rCOX that retains Arg120 is r57 which should synthesize a product that inhibits EMCV replication that differs from r50 and r44's products. While r50 produces a lipoxin that aids in EMCV replication. Also, due to confocal images showing an autophagy activation pattern for endogenous ATG9 when r44 is intranuclear and studies showing EMCV replication is dependent on autophagy activation we determine the r44 protein causing EMCV maturation to double could indicate r44 activates autophagy.

Finally, comparison of rcCOXs with prokaryotic and plant CLPs showed striking similarities in both alpha helical and catalytic residue conservation even with low residue homology (typically ˜30%). These secondary structure features are highly selected for in organismal survival against both invading pathogens and cellular stress. Our work presents more roles COX genes play in innate immunity and further research is needed to determine the effects of NSAIDs and other anti-pyretic drugs on this new role of COX.

### Materials and Methods

### Cell Culture

CHO cells were from ATCC and grown in DMEM/F12 50:50 medium (Corning) with 10% fetal bovine serum (FBS) and 1% penicillin/streptomycin (Gibco) at 37 C and 5% C02 in humid environment and used at passages below 20. Cells were passaged passaged by washing the cells twice in phosphate buffered saline ([PBS]) and trypsinized (0.05% trypsin from Sigma-Aldrich) until cells detached and seeded onto new plates.

### Plasmid DNA Purification

We used Genelute plasmid mini-prep kit (Sigma-Aldrich) for purification of plasmid DNA from transformed DH5a E. coli cells. Transformed cells were grown in LB broth ( ) overnight at 37 C and 250 rotation. After incubation, cells were pelleted at 3000×g for 10 minutes. The LB broth was removed and pellets were resuspended in 200 uL of Resuspension buffer. Cells were lysed by the addition 200 uL of Lysis buffer. After inverting tubes twice 350 uL of Neutralization buffer was added and samples were again inverted. Cell debris was pelleted in microcentrifuge (Eppendorf 1517C) for 10 minutes at 14,000 rpm. Supernatant was removed and transferred to mini-prep column provided by kit and centrifuged again for 1 minute at 14,000 rpm. Then 750 uL of Wash buffer was added to column and centrifuged again as before. Column was dried by centrifugation for 2 minutes at 14,000 rpm. Elution buffer was added to dried columns and centrifuged at 14.000 rpm for 1 minute. DNA concentration was measured using Take3 module for the Synergy H4 Hybrid plate reader.

### DC Protein Assay

DC protein assay kit provided by Bio-Rad. For protein measurement, 5 μL sample was placed into wells of a 96-well plate. Then 25 μL of Reagent A, prepared by addition of 20 μL of Reagent S into 1 mL of Reagent A, was added to each sample and the reaction started by the addition of 200 μL of Reagent B and allowed to incubate for 15 minutes at rt. Protein was measured at an absorbance of 750 nm using Synergy H4 Hybrid plate reader. The measurements were compared to protein standard prepared by serial dilution of 2 mg/mL of BSA (Bio-Rad) to 0.05 mg/mL and then measured as described above.

### Transient Transfection

Transient transfection were performed as described before. Briefly, CHO cells were seeded onto 6-well plates at a confluency of 70% in DMEM/F12 media with 10% FBS and allowed to incubate overnight at 37 C and 5% CO2. After incubation, DNA was prepared by adding 1500 ng of DNA to DMEM/F12 to a total volume of 100 μL. Then 100 μL of lipofectamine solution (10 μL of lipofectamine [Invitrogen] into 90 μL of DMEM/F12) was added and allowed to incubate at rt on rotator for 20 minutes. Cells were washed twice with DMEM/F12 and 600 μL of media added to cells. Prepared lipofectamine/DNA solution was added to washed cells, total volume is 800 μL, and allowed to incubate for 2 hours at 37 C. After incubation, 800 μL of DMEM/F12 media with 20% FBS was added and cells left to incubate at 37 C for 24 hours

### Confocal Microscopy

After cells were transfected as described above, cells were washed thrice with cold PBS and fixed with 4% paraformaldehyde for 15 minutes at room temperature. Fixative was then aspirated and samples were again washed 3 times with cold PBS. Samples were blocked using block solution (PBS with 1% Tween 20 and 1% goat serum) for 30 minutes. After blocking, samples were again washed 3 times with cold PBS and probed with primary antibodies in block solution overnight. Antibody solution was removed and samples washed 3 times with cold PBS for 10 minutes each with slight agitation. After washes, samples were probed with secondary antibody prepared in PBS with 1% Tween 20 for 2 hours at room temperature. Then samples were again washed 3 times for 10 minutes each in PBS. Cover slips were then mounted on slides using Fluoromount-G. Slides were sequentially scanned using filter settings for DAPI, TRITC, FITC, or Texas red using an Olympus FluoView FV000 confocal laser scanning microscope.

### Site-Directed Mutagenesis

Site-directed mutagenesis were done using the Invitrogen kit Geneart site-directed mutagenesis. Primers were prepared where the forward primer contained the mutation while the reverse primer contained at least 12 nucleotides on the 5′ end overlap the 5′ end of the forward primer. PCR reaction was performed using platinum Pfx (Invitrogen) following the protocol provided with 100 ng of DNA template. Amplification was performed using GeneAMP 9700 PCR system thermocycler (Applied Biosystems). After the polymerase was heat activated at 94 C for 5 minutes, samples were amplified for 18 cycles where each cycle was the following: 30 seconds at 94 C, 30 seconds at melting temperature of the primers (usually 57 C), and amplicons extended at 68 C for 1 minute for every 1000 bp of DNA template. After 18 cycles, complete synthesis was assured by an extra 5 minutes of reaction at 68 C. Amplicons were then separated from template by DpnI digestion (New England Bio) and electrophoresed on a 1% agarose gel in TBE buffer (0.18 M Tris, 0.1 M boric acid, 2 mM EDTA) with 0.0024% ethidium bromide and visualized using UV light. The visualized amplicons were then excised from the gel and purified using Qiagen Gel Extraction kit.

Purified amplicons were then recombined using Geneart site-directed mutagenesis module (Invitrogen) where amplicon was added to 5× reaction buffer, 10× recombination enzyme, and Dnase/Rnase free water to a final volume of 10 μL. The reaction was allowed to run for 10 minutes at rt. After the reaction, circularized amplicon was then transformed into chemical competent DH5α E. coli cells (Invitrogen).

Transformation followed protocols provided by Invitrogen. Briefly, DNA incubated with the competent cells for 30 minutes on ice. After 30 minute incubation, cells were heat shocked at 42 C for 22 seconds and placed back on ice for 2 minutes. SOC media, provided by the kit, was added at 250 μL for every 50 μL of E. coli cells. Cells were then incubated for 1 hour at 37 C on rotator. After 1 hour incubation, bacteria were inoculated onto LB plates with 100 mg/L ampicillin and cultured at 37 C overnight. The following day, single colonies were picked and cultured in LB broth overnight at 37 C. DNA plasmids were then purified from bacteria described above.

### Plaque-Forming Assay

CHO cells were seeded to the night before infection to complete confluence. Cells were washed twice with DMEM F/12 media before infection. Virus were placed on top of cells and allowed to absorb for 1 hour angling the cells every 10 minutes to ensure cells were fully covered. After adsorption the media was remove and cells washed twice with DMEM F/12 media. Agar at 2% in PBS was melted and mixed with warm DMEM/F12 with 20% FBS and then laid on top of the cells. The virus was allowed to replicate for 36 hours and then 5 mg/mL of MTT in PBS was added to visualize the cells. Plaques were counted using light microscope.

### Immunoblotting

Samples were denatured at 65 C in 4× Sample buffer. Denatured samples were electrophoresed on 10% polyacrylamide (PAGE) gels with 0.47% bis-acrylamide gels using Running buffer (100 mM Tris, 750 mM glycine, 1% (w/v) SDS) at constant milliamp of 15 per gel until Bio-Rad molecular weight markers were significantly separated. Proteins in gel were then transferred to Bio-Rad nitrocellulose membrane (0.2 μm) at a constant voltage of 100V for 75 minutes. Transfer buffer (100 mM Tris, 750 mM glycine, 20% (v/v) MeOH) was kept cold throughout the transfer. After transfer, membranes were blocked using 2.5% milk in PBS for at least 1 hour. Primary antibodies were incubated with membranes in 0.5% milk in PBS overnight on rotator at 4 C. After incubation, membranes were washed 3 times in PBS+0.1% (v/v) Tween 20 (PBST) for 5 minutes at rt. After washes, secondary anti-bodies labeled with IR dye (LI-COR Biosciences) in PBST were incubated with the membrane for 1 hour at rt. Blots were again washed 3 times in PBST and dried before scanning. Membranes were scanned using an Odyssey scanner (LI-COR Biosciences). Primary antibodies used throughout this work were anti-FLAG (Santa Cruz Biotechnology, Siga Aldrich), anti-Myc (Abcam), anit-HA (Santa Cruz.,

### BIBLIOGRAPHY

Sarkar, S.; et. al. Impaired Autophagy in the lipid-storage disorder Niemann-Pick Type C1 disease. Cell Reports, 2013, 5, 1302-1315.
Simmons, D. L.; Botting, R. M.; and Hla, T. Cyclooxygenase Isoenzymes: The Biology of Prostaglandin Synthesis and Inhibition. Pharmo. Rev. 2004, 56, 387-437.
Zhang, Y.; Li, Z; Ge, X.; Guo, X.; and Yang, H. Autophagy promotes the replication of encephalomyocarditis virus in host cells. Autophagy, 2011, 7, 613-628.
Oberste, M. S.; Gotuzzo, E.; Blair, P.; Nix, W. A.; Ksiazek, T. G.; Comer, J. A.; Rollin, P.; Goldsmith, C. S.; Olson, J.; and Kochel, T. J. Human Febrile Illness caused by encephalomyocarditis virus infection, Peru. Emerging Infect. Dis. 2009, 15, 640-646.
Beretta, L.; Svitkin, Y. V.; and Sonenberg, N. Rapamycin stimulates viral protein synthesis and augments the shutoff of host protein synthesis upon picornavirus infection. J. Virology, 1996, 70, 8993-8996.
Young, A. R. J.; Chan, E. Y. W.; Hu, X. W.; Kochi, R.; Crawshaw, S. G.; High, S; Hailey, D. W.; Lippincott-Schwartz, J.; and Tooze, S. A. Starvation and ULK1-dependent cycling of mammalian ATG9 between the TGN and endosomes. J. of Cell Sci, 2006, 119, 3888-3900.
Lin, P.; Fischer, T.; Weiss, T.; and Farquhar, M. G. Calnuc, an EF-hand Ca2+ binding protein, specifically interacts with the C-terminal α5-helix of Gαi3. PNAS, 2000, 97, 674-679.
Weiss, T. S.; Chamberlain, C. E.; Takeda, T.; Lin P.; Hahn, K. M.; and Farquhar, M. G. Gαi3 binding to calnuc on golgi membranes in living cells monitored by fluorescence resonance energy transfer of green fluorescent protein fusion protein. PNAS. 2001, 98, 14961-14966.
Baliff, B. A.; Micek, N. V.; Barratt, J. T.; Wilson, M. L.; and Simmons, D. L. Interaction of cyclooxygenases with an apoptosis- and autoimmunity-associated protein. PNAS, 1996, 93, 5544-5549.
Lin, P.; Li-Niculescu, H.; Hofmeister, R.; McCaffery, J. M.; Jin, M.; Hennemann, H.; McQuistan, T.; Vries, Luc De; and Farquhar, M. G. The mammalian calcium-binding protein, nucleobindin (CALNUC), is a golgi resident protein. J. Cell Bio. 1998, 141, 1515-1527.
Lu, X.; Xie, W.; Reed, D.; Bradshaw, W. S.; and Simmons, D. L. Nonsteroidal anti-inflammatory drugs cause apoptosis and induce cyclooxygenase in chicken embryo fibroblasts. PNAS, 1995, 92, 7961-7965.
Kemball, C. C.; Alirezaei, M.; Flynn, C. T.; Wood, M. R.; Harkins, S.; Kiosses, W. B.; and Whitton, J. L. Coxsackievirus infection induces autophagy-like vesicles and megaphagosomes in pancreatic acinar cells in vivo. J Virolo, 2010, 84, 12110-12124.
Zhang, Y. I-TASSER server For protein 3D structure prediction. BMC Bioinformatics, 2008, 9.
Roy, A.; Kucukural, A.; and Zhang Y. I-TASSER: a unified platform for automated protein structure and function prediction. Nature Protocols, 2010, 5, 725-738.
Roy, A.; Yang, J.; Zhang, Y. COFACTOR: an accurate comparative algorithm for structure-based protein function annotation. Nucleic Acids Res. 2012, 40, W471-W477.
Xie, W. L.; Chipman, J. G.; Robertson D. L.; Erickson, R. L.; and Simmons, D. L. Expression of a mitogen-responsive gene encoding prostaglandin synthase is regulated by mRNA splicing. PNAS, 1991, 88, 2692-2696.
Simmons, D. L.; Levy, D. B.; Yannoni, Y.; and Erikson, R. L. Identification of a phorbol ester-repressible v-src-inducible gene. PNAS, 1989, 86, 1178-1182.
Simmons, D. L.; Botting, R. M.; Robertson, P. M.; Madsen, M. L.; and Vane, J. R. Induction of an acetaminophen-sensitive cyclooxygenase with reduced sensitivity to nonsteroid anti-inflammatory drugs. PNAS, 1999, 96, 3275-3280.
Shimokawa, T. and Smith, W. L. Essential histidines of prostaglandin endoperoxide synthase: His-309 is involved in heme binding. JBC, 1991, 266, 6169-6173.
Deretic, V. and Levine, B. Autophagy, immunity, and microbial adaptations. Cell Host and Microbe, 2009, 5, 527-549.
Zamocky, M. and Obinger, C. Molecular phylogeny of heme peroxidases. In Biocatalysis based on heme peroxidases; Torres, E. and Ayala M., Ed.; Springer-Verlag: Berlin Heidelberg, 2010; p 7.

### Example 3

### Chemical Assays for Drug Screening

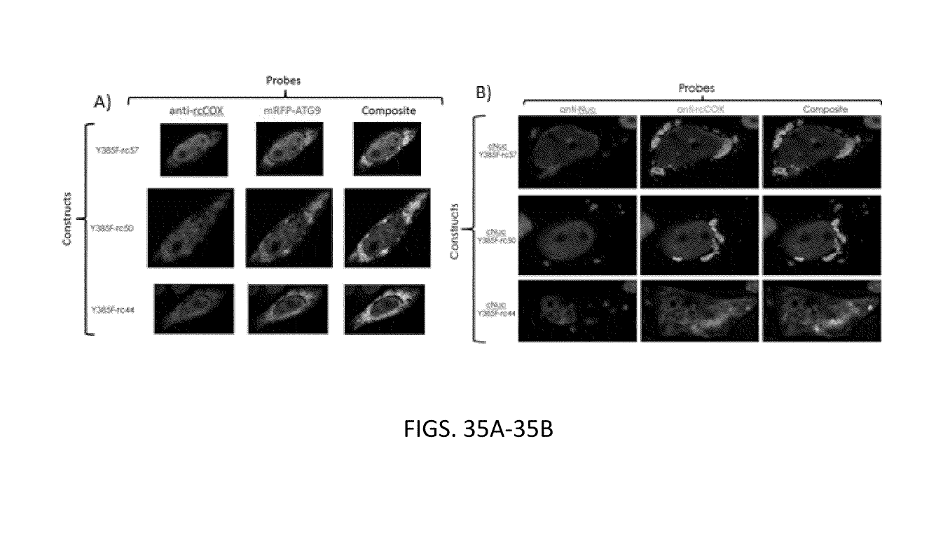

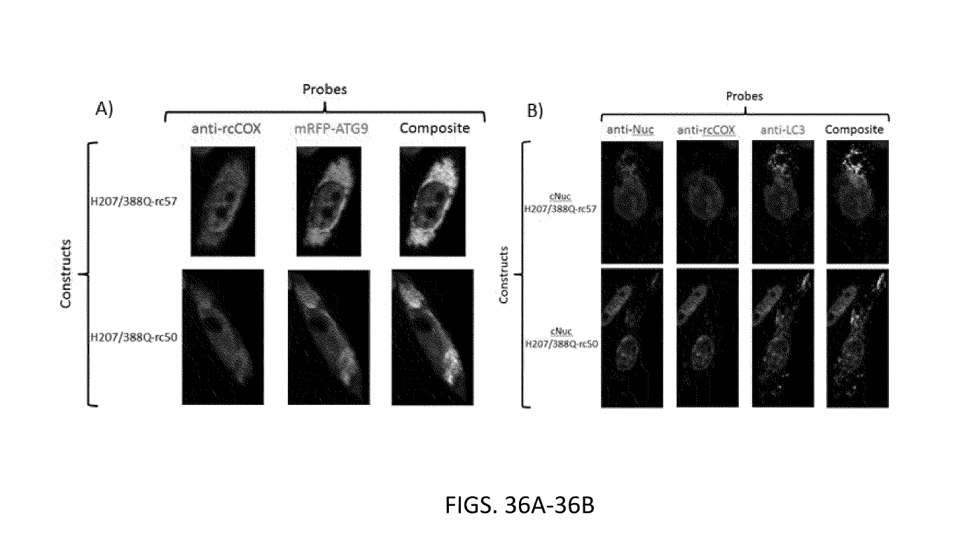

The finding that recoded cyclooxygenase proteins (rc57, rc50, and rc44) bind to the ATG9 complex and regulate membrane transport from the ATG9 vesicle to the phagophore/autophagosome, led us to test whether these proteins possessed enzymatic activity, and whether this enzymatic activity was essential for ATG9 vesicle fusion with the phagophore. Therefore, the tyrosine that forms the reactive tyrosyl radical in all cyclooxygenases (often referred to as tyrosine 385, using the ovine COX-1 primary sequence as reference) was mutagenized to a phenylalanine. This is a change of a single hydroxyl group, leaving a structurally similar aromatic amino acid but preventing the formation of a tyrosyl radical. Similarly, histidine 388, which constitute the proximal ligand to heme of cyclooxygenase enzymes, and histidine 207 which provides the distal ligand, were mutagenized to glutamine in separate assays. Both tyrosine- and histidine-mutagenized proteins yielded similar phenotypes in transfected cells: recoded cyclooxygenase proteins bound ATG9 vesicles, but these vesicles did not form autophagosomes or megaautophagosomes (when cytosolic nucleobindin was co-transfected) (FIG. 35 and FIG. 36). Thus, a redox mediated enzymatic activity of recoded COX proteins requiring heme and a tyrosyl radical is essential for fusion of ATG9 vesicles with the phagophore and/or for phagophore maturation into autophagosomes. The blockade of this pathway with small molecule therapy would be useful in all areas where autophagy has been implicated in disease including, as examples, cancer, neurodegenerative disease, immunity, and inflammation.

The finding that a redox enzymatic reaction involving heme and a tyrosyl radical at 385 presents a potential druggable enzymatic target to modulate autophagy pathways where these unique COX-related proteins function. Since autophagy and COX (i.e. COX-1 and COX-2) proteins are involved in innate immunity and inflammation, these physiological and pathological processes, such as xenophagy, are particularly attractive targets.

To identify assays for drug targeting of these unique recoded COX-related proteins we have used the two enzymatic reactions associated with cyclooxygenases as predictors for the types of enzyme activities carried out by rc57, rc50, and rc44. The first reaction is the oxygenation of lipid substrate, typically an unsaturated fatty acid, to form an oxylipin. Many diverse types of oxylipins are now known, but cyclooxygenases are most closely associated with their production of prostaglandins. Because these recoded COX-related proteins lack glycosylation and other features of cyclooxygenases, they likely make products other than prostaglandins, which we determined to be the case (see below). In order for cyclooxygenase-like enzymes to make oxylipins, the enzymes require a peroxidase activity that occurs at an active site that is distinct from that which produces the oxylipin. The interplay between the peroxidase and oxylipin active site is required for oxylipin production.

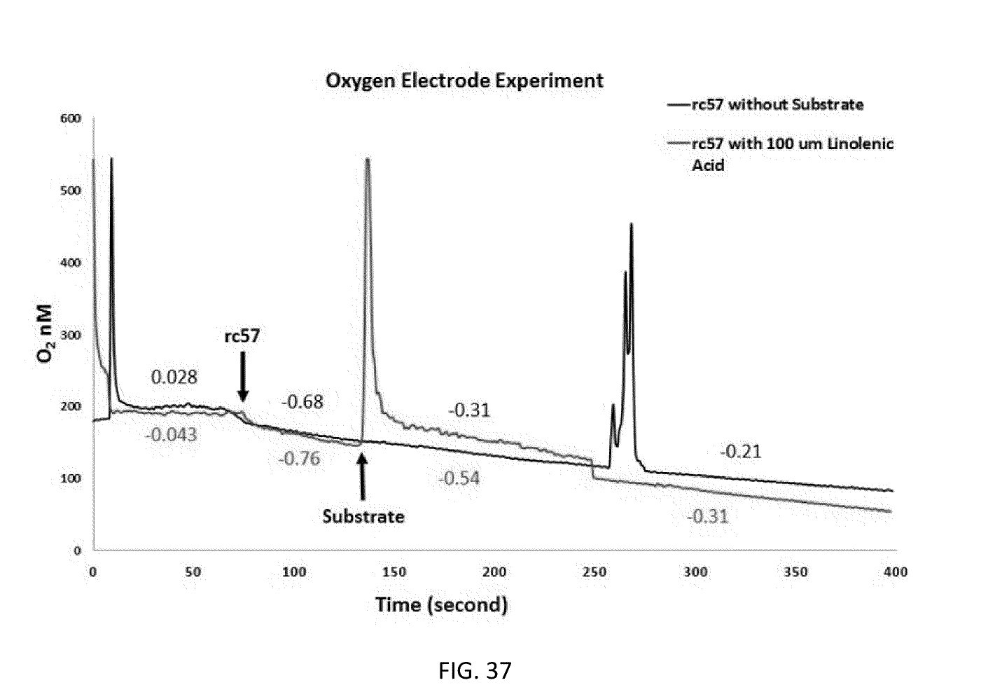

We first tested recombinant rc57, rc50, and rc44 for prostaglandin synthesis by standard measurement of prostaglandin E2 in a radioimmunoassay. All three enzymes fail to make prostaglandins, demonstrating that they are enzymatically distinct from cyclooxygenases 1 and 2. Next we tested for oxylipin production by challenging recombinant rc57 with various unsaturated fatty acids as potential substrates and measuring for a decrease in solvent oxygen levels as oxygen became integrated into the fatty acids tested. The assay details are described in the section immediately below. The addition of lenolenic acid produced a marked decrease in solvent oxygen levels in the assay, indicating this fatty acid as a potential substrate of rc57 (FIG. 37). This finding also demonstrated that rc57 is an oxylipin synthase and, therefore, possesses intrinsic peroxidase activity as well.

From this finding the following methods can be developed to test drugs against recombinantly produced rc57, rc50, and rc44 (and their human orthologs) in high-throughput drug testing, a) Identification of oxylipin products generated by these enzymes can be used to produce specific anti-oxylipin antibodies for rapid enzyme-linked immunosorbant assays or radioimmunoassays. In this assay, drug would be applied to the recombinant system and inhibition of the oxylipin product would be measured either by ELISA or by radioimmunoassay; b) Oxylipin products can be similarly measured after drug treatment in recombinant enzyme systems by nuclear magnetic spectroscopy, mass spectrometric, chromatographic elution (e.g. HPLC, capillary electrophoresis or chromatography, gas chromatography, etc.), radioisotopic and other analytical methods of measuring small molecules. c) Fluorescence-based methods of measuring redox reactions for cyclooxygenase and other redox enzymes are known, and can be applied to recombinant recoded COX proteins, including but not limited to oxygenation of fluorescent or proto-fluorescent substrate molecules, 1 d) Peroxidase enzymes using chromogenic or fluorescent substrates such as guaiacol, or TMPD can be used in recombinant assays to test for inhibition or modulation of the peroxidase enzyme active site.

In the above assays we seek for molecules that either stimulate or inhibit the redox activity of these proteins because both stimulators and inhibitors may be important in specific medical situations.

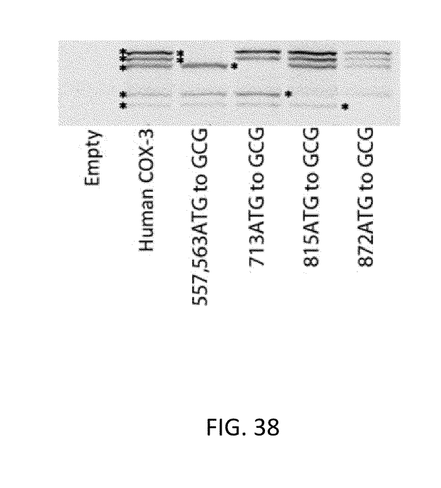

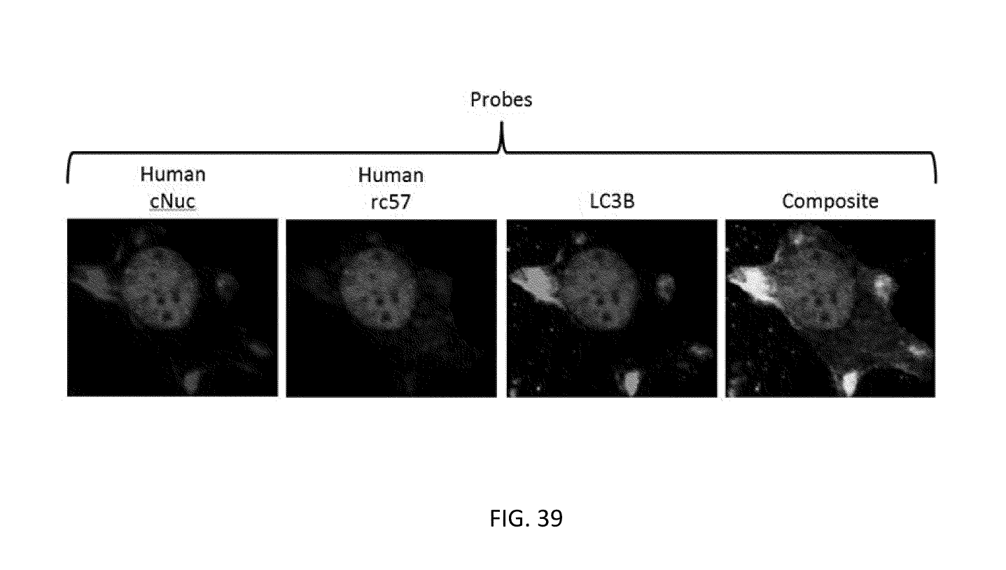

Because the human COX-3/COX1b mRNA is recoded in a fashion very similar to that of rat, and because the human orthologs of the rat proteins localize to autophagosomes in a fashion similar to rat (FIG. 38 and FIG. 39 and shown in reference 2), we also claim the same methods for testing the human orthologs of the rat proteins we have identified.

Recombinant Assay Details.

Linearized rc57 construct with the T7 promoter region, T7 terminator region, and ribosome binding site was prepared by polymerase chain reaction (PCR) using rc57 transcript in pcDNA 3.1 as a template. 2 Recombinant rc57 was then synthesized from the linearized rc57 construct using Escherichia coli-based cell-free protein synthesis by the method of the Bundy laboratory. 3 Once enzyme was synthesized, activity was measured by 02 consumption using a Hanatech Oxy/Ecu. Reaction buffer (PBS and 1 μM hematin) of 1.7 mL was heated to 37° C. and stirred using a magnetic stir bar at 100 rpm. O2 levels were measured for 1 minute before the introduction of 200 μL rc57 lysate. Once rc57 lysate is added, O2 levels were again measured for 1 minute to serve as background level of O2 consumption. Then 100 μL of substrate or PBS was added and O2 consumption was measured for an additional 4 minutes. Rates of consumption were calculated by plotting linear regressions using Excel.

### BIBLIOGRAPHY

1. Bonner, A and Fry, M. R. Development of a fluorescent based assay to detect cyclooxygenase inhibitory activity of a delta-lactone derivative. www.dep.anl.gov/p_undergrad/ugsymp/2012/abstracts/41.html
2. Hunter, J. C. Multiple Recoding Mechanisms Produce Cyclooxygenase and Cyclooxygenase-Related Proteins from Frameshift-Containing COX-3/COX-1b Transcripts in Rat and Human. Ph.D. Dissertation, Brigham Young University, Provo, Utah, December 2012.
3. Shrestha P.; Holland, T. M.; and Bundy, B. C. Streamlined Extract Preparation for Escherichia coli-based Cell-Free Protein Synthesis by Sonication or Bead Vortexing Mixing. BioTechniques. 2012, 53, 163-174.

What is claimed is:

1. A method for modulating autophagy in a cell comprising contacting one or more cells that expresses COX-3 with an effective amount of an agent that modulates the expression level and/or enzymatic activity of COX-3 or an isoform thereof.

2. The method of claim 1 wherein modulating autophagy comprises inhibiting autophagy.

3. The method of claim 2 wherein inhibiting autophagy inhibits viral replication.

4. The method of claim 2 wherein the agent specifically inhibits the expression level and/or enzymatic activity of COX-3 protein or the isoform thereof.

5. The method of claim 4 wherein the agent interferes with COX-3 protein interaction with nucleobindin (Nuc), thereby inhibiting autophagy.

6. (canceled)

7. (canceled)

8. (canceled)

9. The method of claim 1 wherein the isoform of COX-3 is selected from the group consisting of r68, r57, r50 or r44.

10. (canceled)

11. (canceled)

12. (canceled)

13. (canceled)

14. (canceled)

15. (canceled)

16. The method of claim 1 wherein modulating autophagy comprises inducing autophagy.

17. The method of claim 16 wherein inducing autophagy inhibits the growth or proliferation of tumor cells.

18. The method of claim 16 wherein inducing autophagy suppresses viral infection.

19. The method of claim 16 wherein the agent specifically increases the expression level and/or enzymatic activity of COX-3 protein or an isoform thereof.

20. (canceled)

21. (canceled)

22. (canceled)

23. (canceled)

24. The method of claim 16 wherein the isoform of COX-3 is selected from the group consisting of r68, r57, r50 or r44.

25. (canceled)

26. (canceled)

27. (canceled)

28. (canceled)

29. A method for inhibiting viral replication comprising contacting a cell that expresses COX-3 with an effective amount of an agent that inhibits the expression level and/or enzymatic activity of COX-3 or an isoform thereof.

30. The method of claim 29 wherein viral replication comprises autophagy-associated viral replication.

31. The method of claim 30 wherein viral replication comprises picornavirus viral replication.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. A method for inhibiting viral infection in a subject comprising administering to the subject an effective amount of an agent that increases the expression level and/or enzymatic activity of COX-3 or an isoform thereof.

45. (canceled)

46. (canceled)

47. (canceled)

48. (canceled)

49. (canceled)

50. The method of claim 44 wherein the agent specifically increases the expression level and/or enzymatic activity of COX-3 protein or the isoform thereof.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. A method of identifying a candidate agent that modulates autophagy in a cell comprising: a) contacting a cell or population of cells that expresses COX-3 protein or an isoform thereof with a candidate autophagy modulating agent; and b) measuring the level of expression and/or enzymatic activity of COX-3 or the isoform thereof, wherein: i) a decrease in expression and/or enzymatic activity of COX-3 protein or the isoform thereof relative to a control cell or population of cells not exposed to said candidate autophagy modulating agent is indicative that said candidate autophagy modulating agent inhibits autophagy; or ii) an increase in expression and/or enzymatic activity of COX-3 protein or isoform thereof relative to a control cell or population of cells not exposed to said candidate autophagy modulating agent is indicative that said candidate autophagy modulating agent induces autophagy.

65. The method of claim 64, further comprising assessing autophagy of said cell or population of cells.

66. A method of identifying a candidate agent that inhibits viral replication comprising: a) providing a composition comprising a COX-3 polypeptide or isoform thereof and a candidate agent; (b) determining whether the candidate agent inhibits the COX-3 polypeptide or isoform thereof; wherein if the candidate agent inhibits the COX-3 polypeptide or isoform thereof, the candidate agent is identified as a candidate agent that inhibits viral replication.

67. The method of claim 66, further comprising assessing the ability of the candidate agent that inhibits viral replication to inhibit viral replication.

68. (canceled)

69. (canceled)

70. (canceled)

71. (canceled)

72. (canceled)

73. (canceled)

74. (canceled)

75. A method of identifying a candidate agent that suppresses viral infection comprising: a) providing a composition comprising a COX-3 polypeptide or isoform thereof and a candidate agent; (b) determining whether the candidate agent induces the COX-3 polypeptide or isoform thereof; wherein if the candidate agent induces the COX-3 polypeptide or isoform thereof, the candidate agent is identified as a candidate agent that suppresses viral infection.

76. The method of claim 75, further comprising assessing the ability of the candidate agent that suppresses viral infection to suppress viral infection.

77. (canceled)

78. (canceled)

79. (canceled)

80. (canceled)

81. (canceled)

82. (canceled)

83. (canceled)

84. (canceled)


SEQUENCE LISTING (70 sequences)

SEQ ID NO: 1 - DNA, 21 bp, Artificial
  primer
tcagagctctgtggatggtcg

SEQ ID NO: 2 - DNA, 14 bp, Artificial
  primer
agccaaattcgttg

SEQ ID NO: 3 - DNA, 14 bp, Artificial
  primer
gtcgccagccgagc

SEQ ID NO: 4 - DNA, 16 bp, Artificial
  primer
accttgcccacagcct

SEQ ID NO: 5 - DNA, 20 bp, Artificial
  primer
ccatgagccggagtctcttg

SEQ ID NO: 6 - DNA, 26 bp, Artificial
  primer
ctgatgtagtcatgtgctgagttgta

SEQ ID NO: 7 - DNA, 18 bp, Artificial
  primer
catgagccgtgagtgcga

SEQ ID NO: 8 - DNA, 26 bp, Artificial
  primer
ctgatgtagtcatgtgctgagttgta

SEQ ID NO: 9 - RNA, 19 nt, Artificial
  siRNA
gguggagccuugaaugcca

SEQ ID NO: 10 - RNA, 19 nt, Artificial
  siRNA
ccugguggagccuugaaug

SEQ ID NO: 11 - RNA, 19 nt, Artificial
  siRNA
cucaucucucuccucugca

SEQ ID NO: 12 - RNA, 19 nt, Artificial
  siRNA
ggaauuguauggagacauu

SEQ ID NO: 13 - RNA, 19 nt, Artificial
  siRNA
gaacauggaccaccacauc

SEQ ID NO: 14 - RNA, 19 nt, Artificial
  siRNA
caagagguuuggcaugaaa

SEQ ID NO: 15 - RNA, 19 nt, Artificial
  siRNA
ggagauggcagcagaguug

SEQ ID NO: 16 - DNA, 16 bp, Artificial
  primer
gtcatgagtcgtgagt

SEQ ID NO: 17 - DNA, 20 bp, Artificial
  primer
gtacaactctccatccagca

SEQ ID NO: 18 - DNA, 19 bp, Artificial
  primer
cagagtcatgagtcgaaga

SEQ ID NO: 19 - DNA, 20 bp, Artificial
  primer
gtacaactctccatccagca

SEQ ID NO: 20 - DNA, 19 bp, Artificial
  primer
ggtggaagaatgggagttg

SEQ ID NO: 21 - DNA, 18 bp, Artificial
  primer
ggtggagaatgggagttg

SEQ ID NO: 22 - DNA, 20 bp, Artificial
  primer
ctcgtgcaaaagcgaagtct

SEQ ID NO: 23 - DNA, 20 bp, Artificial
  primer
tacccaagcctctcctcaga

SEQ ID NO: 24 - DNA, 20 bp, Artificial
  primer
aagggcaaggatggtagatg

SEQ ID NO: 25 - DNA, 20 bp, Artificial
  primer
cctccacaccaaagacaaac

SEQ ID NO: 26 - DNA, 20 bp, Artificial
  primer
gcagagaagaccaaggaagg

SEQ ID NO: 27 - DNA, 20 bp, Artificial
  primer
catgccacaatcacaacgta

SEQ ID NO: 28 - DNA, 24 bp, Artificial
  primer
agactccttcactcatgacgactc

SEQ ID NO: 29 - DNA, 17 bp, Artificial
  primer
actccttcctgcagagg

SEQ ID NO: 30 - DNA, 21 bp, Artificial
  primer
tgtgccagattaccctggaga

SEQ ID NO: 31 - DNA, 23 bp, Artificial
  primer
gggaactggttttcaactggagg

SEQ ID NO: 32-33 - skipped (2 entries)

SEQ ID NO: 34 - DNA, 33 bp, Unknown
  partial rat COX-3 sequence
agaagagccagccgttggcgttgcacatccatc

SEQ ID NO: 35 - DNA, 39 bp, Unknown
  partial rat COX-1 sequence
aacaccagaagagccagccgttggcgttgcacatccatc

SEQ ID NO: 36 - DNA, 50 bp, Unknown
  partial rat COX-1 sequence
agtgctggagtaacaccagaagagccagccgttggcgttgcacatccatc

SEQ ID NO: 37 - DNA, 11 bp, Unknown
  partial rat COX-1 sequence
gcacatccatc

SEQ ID NO: 38 - protein, 127 aa, Unknown
  COX-3 peptide sequence
MSRESDPSGAPTRPGIRWPAGGALNVRLNSLFPLQEGVSRSSFPCCCSCCCSHHPRYCSQMLGYPHQSIPVVTIHARTRVSVSASASTTTNVTVLARATRAPTVLSLRSGPGFGVPCGPAPHSPISC

SEQ ID NO: 39 - protein, 631 aa, Unknown
  COX-3 peptide sequence
VRPQRCPHAPRNSVACRRSLERQAQLSLPSARRSLSLQFPLLLLLLLLPPPPVLLTDAGVPSPVNPCCYYPCQNQGVCVRFGLDHYQCDCTRTGYSGPNCTIPEIWTWLRSSLRPSPSFTHFLLTHGYWIWEFVNATFIREVLMRLVLTVRSNLIPSPPTYNTAHDYISWESFSNVSYYTRILPSVPKDCPTPMGTKGKKQLPDIHLLAQRLLLRREFIPAPQGTNVLFAFFAQHFTHQFFKTSGKMGPGFTKALGHGVDLGHIYGDSLERQYHLRLFKDGKLKYQVLDGEVYPPSVEQASVLMRYPPGVPPEKQMAVGQEVFGLLPGLMLFSTIWLREHNRVCDLLKEEHPTWDDEQLFQTTRLILIGETIKIIIEEYVQHLSGYFLQLKFDPELLFRAQFQYRNRIALEFNHLYHWHPLMPDSFQVGSQEYSYEQFLFNTSMLVDYGVEALVDAFSRQRAGRIGGGRNFDYHVLHVAEDVIKESREMRLQSFNEYRKRFGLKPYTSFQEFTGEKEMAAELEELYGDIDALEFYPGLMLEKCQPNSLFGESMIEMGAPFSLKGLLGNPICSPEYWKPSTFGGDVGFNIVNTASLKKLVCLNTKTCPYVSFRVPDYPGDDGSVFVRPSTEL

SEQ ID NO: 40 - DNA, 33 bp, Unknown
  COX sequence
atccgagagatgctcatgcgcctggtactcaca

SEQ ID NO: 41 - DNA, 33 bp, Unknown
  COX sequence
atccgagaagtactcatgcgcctggtactcaca

SEQ ID NO: 42 - DNA, 33 bp, Unknown
  COX sequence
atccgagaagtactcatgcgcctggtactcacg

SEQ ID NO: 43 - DNA, 33 bp, Unknown
  COX sequence
atccgggacacgctcatgcgtctggtactcaca

SEQ ID NO: 44 - DNA, 33 bp, Unknown
  COX sequence
atccgggacaagctcatgcgtctggtactcaca

SEQ ID NO: 45 - DNA, 33 bp, Unknown
  COX sequence
ctcagggattggctcatgcggaaagtgctcaca

SEQ ID NO: 46 - DNA, 33 bp, Unknown
  COX sequence
cttcgaaatgcaattatgagttatgtgttgaca

SEQ ID NO: 47 - DNA, 33 bp, Unknown
  COX sequence
ctgcgaagtttaatcatgaaatatgtgctgaca

SEQ ID NO: 48 - DNA, 33 bp, Unknown
  COX sequence
cttcggaattcaatcatgagatacgtgttgacg

SEQ ID NO: 49 - DNA, 33 bp, Unknown
  COX sequence
ctgcgaaatatgattatgagatatgtgttgacg

SEQ ID NO: 50 - DNA, 33 bp, Unknown; identical to SEQ ID NO: 49
  COX sequence

SEQ ID NO: 51 - DNA, 33 bp, Unknown
  COX sequence
ctgagggatggcataatgcgctatatcctgttg

SEQ ID NO: 52 - DNA, 33 bp, Unknown
  COX sequence
gattgccccacacccatgggaaccaaagggaag

SEQ ID NO: 53 - DNA, 33 bp, Unknown
  COX sequence
gactgccccacacccatggggaccaaagggaag

SEQ ID NO: 54 - DNA, 33 bp, Unknown
  COX sequence
gactgtcccacacccatgggaaccaaagggaag

SEQ ID NO: 55 - DNA, 33 bp, Unknown
  COX sequence
gactgtcccacgcccatggacaccaaagggaag

SEQ ID NO: 56 - DNA, 33 bp, Unknown
  COX sequence
gactgtcccacgcccatgggcaccaaagggaag

SEQ ID NO: 57 - DNA, 33 bp, Unknown
  COX sequence
gactgtcctacaccaatgggaaccaaaggaaaa

SEQ ID NO: 58 - DNA, 33 bp, Unknown
  COX sequence
gattgcccgactcccttgggtgtcaaaggtaaa

SEQ ID NO: 59 - DNA, 33 bp, Unknown
  COX sequence
gactgcccaactcccatgggtgtgaagggaaat

SEQ ID NO: 60 - DNA, 33 bp, Unknown
  COX sequence
gactgcccaactcccatgggtgtgaaaggaaat

SEQ ID NO: 61 - DNA, 33 bp, Unknown
  COX sequence
gactgcccaacacccatgggtgtgaaagggagg

SEQ ID NO: 62 - DNA, 33 bp, Unknown; identical to SEQ ID NO: 61
  COX sequence

SEQ ID NO: 63 - DNA, 33 bp, Unknown
  COX sequence
aactgcccaacgcctgatctcccaaatgccaag

SEQ ID NO: 64 - DNA, 33 bp, Unknown
  COX sequence
aaaacttctggcaagatgggtcctggcttcacc

SEQ ID NO: 65 - DNA, 33 bp, Unknown
  COX sequence
aagacctctggaaagatgggtcctggctttacc

SEQ ID NO: 66 - DNA, 33 bp, Unknown; identical to SEQ ID NO: 65
  COX sequence

SEQ ID NO: 67 - DNA, 33 bp, Unknown
  COX sequence
aaaacttccggcaagatgggtcctggcttcacc

SEQ ID NO: 68 - DNA, 33 bp, Unknown; identical to SEQ ID NO: 64
  COX sequence

SEQ ID NO: 69 - DNA, 33 bp, Unknown
  COX sequence
aagactcacaaccgtgtggggctcgggttcaca

SEQ ID NO: 70 - protein, 5 aa, Artificial
  flag tag
DDDDK